(19) United States
(12) Patent Application Publication
(10) Pub. No.: US 2024/0413999 A1
(43) Pub. Date: Dec. 12, 2024

(54) TECHNOLOGIES FOR CREATING NON-FUNGIBLE TOKENS FOR ELECTRONIC HEALTH RECORDS

(71) Applicant: FORTIOR SOLUTIONS, LLC, Hillsboro, OR (US)

(72) Inventors: James Frederick ROBELL, Beaverton, OR (US); James G. HART, Hillsboro, OR (US); Qui Quoc HOANG, Portland, OR (US); Viet Quoc HOANG, Portland, OR (US)

(21) Appl. No.: 18/402,344

(22) Filed: Jan. 2, 2024

Related U.S. Application Data
(60) Provisional application No. 63/437,074, filed on Jan. 4, 2023.

(51) Int. Cl. H04L 9/32; G16H 10/60; H04L 9/00

(52) U.S. Cl. CPC H04L 9/3213; G16H 10/60; H04L 9/50

(57) ABSTRACT

The present disclosure generally relates to the fields of computing, data processing, cryptography, blockchain technologies, tokenization and non-fungible tokens (NFTs), identity verification, account verification, information security (InfoSec), and fraud prevention technologies, and in particular, to technologies for creating NFTs for electronic health records (EHR). An EHR NFT service receives a set of user data items associated with a user, and verifies each user data item of the set of user data items. The EHR NFT service generates a set of hashes, wherein each hash in the set of hashes corresponds to a verified user data item in the set of user data items. The EHR NFT service mints an EHR NFT that includes each hash in the set of hashes and updates a smart contract to include the EHR NFT. Other embodiments may be described and/or claimed.

### RELATED APPLICATIONS

The present application claims priority to U.S. Provisional App. No. 63/437,074 filed Jan. 4, 2023 (“074”), the contents of which is hereby incorporated by reference in its entirety.

### FIELD

The present disclosure generally relates to the fields of computing, data processing, cryptography, blockchain technologies, tokenization and non-fungible tokens (NFTs), identity verification, account verification, information security (InfoSec), and fraud prevention technologies, and in particular, to technologies for creating NFTs for electronic health records (EHR) and/or electronic medical records (EMR).

### BACKGROUND

The background description provided herein is for the purpose of generally presenting the context of the disclosure. Unless otherwise indicated herein, the materials described in this section are not prior art to the claims in this application and are not admitted to be prior art by inclusion in this section.

An electronic health record (EHR) and/or electronic medical record (EMR) is an electronic version of a patient's medical history, and may include all of the key administrative clinical data relevant to that person's care under a particular provider, including demographics, progress notes, problems, medications, vital signs, past medical history, immunizations, laboratory data, radiology reports, and/or the like. An EHR usually contains results of clinical and administrative encounters between a healthcare provider (HP) and a patient that occur during episodes of patient care. Consequently, EHRs reflect the practice style, job function, knowledge and skill of the providers who create it. For instance, EHRs can include data structures and data elements that reflect those HPs' systems.

EHRs can be used to automate access to information and have the potential to streamline HP workflows. Furthermore, EHRs have the ability to support other care-related activities directly or indirectly through various interfaces, including evidence-based decision support, quality management, outcomes reporting, and medical research. However, existing EHRs are maintained by individual HPs. This creates compatibility and/or interworking issues making it difficult to facilitate the potential benefits promised by EHRs, such as streamlining workflows and supporting other care-related activities.

### BRIEF DESCRIPTION OF THE DRAWINGS

In the drawings, which are not necessarily drawn to scale, like numerals may describe similar components in different views. Like numerals having different letter suffixes may represent different instances of similar components. Some examples are illustrated by way of example, and not limitation, in the figures of the accompanying drawings in which:

FIGS. 1, 2, and 3 depict example EHR ecosystems;

FIG. 4 depicts an example EHR NFT platform;

FIGS. 5 and 6 depict example NFT EHR procedures;

FIG. 7 depicts example processes for practicing various aspects of the present disclosure;

FIG. 8 depicts elements of an example NFT/blockchain service;

FIG. 9 depicts components of an example NFT ID engine;

FIG. 10 depicts an example procedure for minting NFT IDs;

FIG. 11 illustrates an example computing system suitable for practicing various aspects of the present disclosure.

### DETAILED DESCRIPTION

### 1. Electronic Health Record (EHR) Non-Fungible Token (NFT) Aspects

The present disclosure provides technologies for using NFT for electronic health records (EHRs), electronic medical records (EMRs) and related purposes. For purposes of the present disclosure, the terms “EMR” and “EHR” may be used interchangeably even though, in some cases, these terms could refer to different concepts.

Healthcare records include personal data, confidential data, and/or sensitive data, and such records are sometimes proprietary to the HPs that create and maintain such records. Medical data is split among many HPs, test companies, insurers, pharmacies, public health agencies, and other entities, leading to frustration, data breaches, and malpractice/deadly mistakes. Currently, there are no unified patient record types or standards. Furthermore, pharmacies have no medical records, just purchased records. Moreover, many HP check-in processes are still based on papers, with human input. These processes are fragmented, siloed, duplicative, and require repetition for each HP. Additionally, telemedicine does not work well without an EHR. Patients are frustrated, treatments are slow and delayed, there is potential loss of lives, and costs increase.

EHRs were created in an attempt to alleviate the issues related to paper-based health records. However, EHRs suffer from the same or similar issues as paper-based health records. For example, there are currently no standardized EHR formats or security mechanisms. Many HPs use their own software (SW) or use third party SW (e.g., MyChart® provided by Epic Systems Corporation and/or the like), but even when using third party SW, systems used by individual HPs are often siloed from one another. Additionally, although the Health Insurance Portability and Accountability Act of 1996 (HIPAA) includes privacy rules that require medical providers to give individuals access to their Protected Health Information (PHI), there are no standard mechanisms allowing patients to access their records on-demand.

The NFT-based mechanisms discussed herein enhances EHR ecosystems by providing an NFT-based form of digitized/tokenized EHR using, for example, private blockchain technologies, smart contracts, and digital wallets. NFT-based mechanisms discussed herein can also be used to connect patients, EHRs, mobile devices, HPs, insurers, and other relevant entities/elements to the same secure database (DB). Additionally, artificial intelligence (AI) and/or machine learning (ML) systems (see e.g., '074 and/or U.S. Provisional App. No. 63/437,081 filed Jan. 4, 2023 (“'081”), the contents of which is hereby incorporated by reference in its entirety) and/or data mining systems can be used to connect the dots from various sources and raise warning flags/indicators when potential issues occur and/or for diagnostic purposes.

The present disclosure at least solves the following problems: reduces multiple EHR processes performed by individual healthcare providers (HPs) to a single EHR process thereby conserving computational and/or network resources; reduces multiple EHR processes in all other HPs in a consortium to zero thereby conserving computational and/or network resources; enables the reuse of previously generated EHR data from individual HPs thereby further reducing computational and/or network resources; converts paper-based health records and/or personal health records (PHRs), and/or other manual EHR creation mechanisms to a digital, automated EHR in NFT forms thereby reducing resource consumption; prevents or reduces the likelihood of mismatched health record data, and detects mismatched data from various EHRs and/or healthcare sources, thereby conserving computational and/or network resources; utilizes smart contracts to only allow certain records to be accessed from authorized HPs and/or other entities/elements thereby conserving computational and/or network resources; enables access of EHRs to researchers, statisticians, and/or AI/ML models anonymously thereby facilitating medical research while improving security and patient privacy; and/or enables access of EHRs to AI/ML diagnostic models/tools to facilitate detection and/or diagnosis.

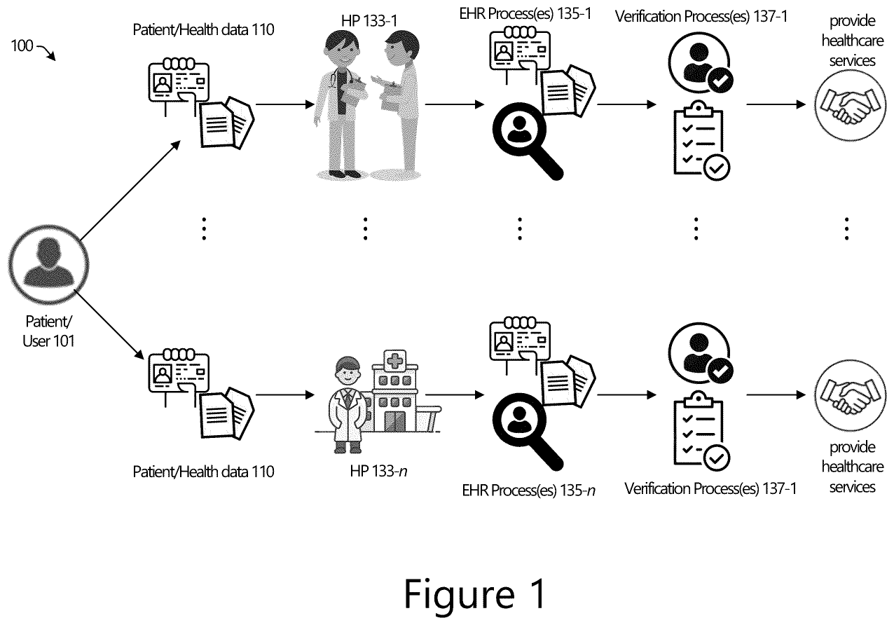

FIG. 1 depicts an example EHR ecosystem 100. Throughout the present disclosure, United States (US)-based EHR ecosystems are used as example use cases; however, the embodiments discussed herein may be applicable to other EHR ecosystems of any geopolitical entity, legal jurisdiction, enterprise or corporate entity, and/or ad-hoc/non-formal entity or group. Additionally, although the present disclosure is based on EHR ecosystems, the embodiments discussed herein can be straightforwardly applied to other secure and/or private records.

In the EHR ecosystem 100, a user (patient) 101 is required to provide their healthcare data (HCD) 110 to each HP 133 (e.g., HP 133-1 to 133-n, where n is a number) they wish to visit. The user 101 may represent a human or one or more computing devices/platforms used by a human. As examples, the HCD 110 can include, for example, user identifiers (IDs), personal information (e.g., full name, date of birth, gender, nationality, citizenship, marital status, residential address, email address, phone number(s), social security number (SSN), NHS number, Canadian health card numbers, Canadian province/territory ID, Australian Medicare Number, Australian individual healthcare ID (IHI), and/or the like), demographic data, medical history, family history, current and/or previous medication(s), allergies, billing information and/or insurance information, identity documents (e.g., passport, driver's license, national ID card, healthcare ID card, insurance card, hospital bracelet, and/or the like), financial data (e.g., occupational data and/or employment records, source(s) of funds and/or income details, HP account statements, and/or the like), credit cards, vital/health records (e.g., birth certificates, marriage licenses, electronic medical records, and/or the like), biometric data, health records/PHRs, and/or the like. Additionally or alternatively, the HCD 110 can include the patient's 101 EHRs, which may be provided by the patient 101 or may be accessed from another entity (e.g., from storage and/or from another HP 133). Examples of the EHR data include personal information, demographic data, medical history, family history, current and/or previous medication(s), allergies, billing/insurance information, immunization status, laboratory test results, radiology images, vital signs, clinical data, progress notes, problems, personal statistics (e.g., age, weight, habits such as smoking history, exercise, diet, and/or the like), medical record number, code sets (e.g. HIPAA/HHS codes sets and/or the like), and/or other administrative and/or clinical data relevant to the user 101 for a particular HP 133. The HCD 110 is used by individual HPs 133 for patient onboarding, patient intake, triage, and/or identity verification purposes. The specific types of HCD 110 needed by individual HPs 133 for these purposes may be based on various factors, such as type of HP 133, jurisdiction(s) in which the user 101 and/or HP 133 operates, the healthcare services being sought/provided, and/or other relevant factors.

Each HP 133 (e.g., one or more of HP 133-1 to 133-n) can be, include, or represent any type of healthcare service provider. As examples, the HPs 133 include public HPs 133, private HPs 133, private insurers, regulators and/or governmental agencies (e.g., World Health Organization (WHO), United States Department of Health and Human Services (HHS), UK National Health Service (NHS), Canada Health, and the like), and/or other entity/org that provides healthcare services, including any of those discussed herein. Additionally, some or all of the HPs 133 can include or operate respective HP platforms/systems, which can include, for example, a hospital information system (HIS), hospital management system (HMS), health information management (HIM) system, health informatics tools, health information technology, and/or the like. The HP platforms/systems can be implemented using suitable database management systems, distributed computing systems, cloud computing technologies, edge networking technologies, and/or other technologies, such as any of those mentioned herein. For purposes of the present disclosure, the terms “HP 133”, “healthcare provider”, “HP system”, “HP platform”, and/or the like may be used interchangeably throughout the present disclosure, and these terms may refer to the HPs 133 themselves, HP systems/platforms used by an individual HP 133 (e.g.,), and/or the one or more users of the HP systems/platforms, unless the context dictates otherwise.

Each HP 133 has its own EHR processes 135. For example, a first HP 133-1 has its own EHR process(es) 135-1, a second HP 133-2 has its own EHR process(es) 135-2 (not shown by FIG. 1), and so forth to an n-th HP 133-n that has its own EHR process(es) 135-n. Additionally, each HP 133 conducts its own patient verification process(es) 137 before proceeding to provide healthcare services to the patient 101. For example, a first HP 133-1 has its own verification process(es) 137-1, a second HP 133-2 has its own verification process(es) 137-2 (not shown by FIG. 1), and so forth to an n-th HP 133-n that has its own verification process(es) 137-n.

The EHR processes 135 and patient verification processes 137 may include, for example, patient onboarding, patient intake, triage, and/or identity verification. Patient onboarding involves processes/procedures by which patients are welcomed and oriented into a HP's 133 practice. Patient intake involves processes/procedures through which HPs 133 collect demographic data, social data, clinical data, consent forms, insurance information, payments, and/or other relevant pieces of information from new and returning patients prior to their visit. Triage involves processes/procedures for prioritizing patients based on their need for treatment. Identity verification involves processes/procedures for confirming or denying that a patient's 101 claimed identity is correct by, for example, comparing their identity data/credentials with those previously proven (and/or associated with known/stored credentials or identity data), and/or otherwise confirming that specified identity verification requirements have been fulfilled through the provision of objective evidence. While some HPs 133 may use automated tools and systems to conduct patient onboarding (e.g., automated patient onboarding software), patient intake, and/or identity verification, in most cases, HPs 133 manually verify and validate individual data items of the HCD 110, usually requiring multiple people or departments for completing various tasks associated with the processes 135, 137.

EHR processes 135 and patient verification processes 137 are performed before proceeding to provide healthcare services to the patient 101. The processes 135, 137 are repeated for each HP 133 that the user 101 visits, and is also repeated for each service/department within the same HP 133 that the user 101 wishes to use (e.g., different practices or providers within a hospital, and the like). This is, in part, because there is little or no EHRs and/or related data shared within departments or practice groups, let alone among multiple different separate hospitals or other HPs 133. These repeated processes 135, 137 increase costs for the HPs 133, as well as the healthcare system as a whole, in terms of both time, money, healthcare resources, and computing resources used for various EHR purposes.

Currently, there are multiple ways to store EHRs, and as mentioned previously, individual HPs 133 maintain their own EHRs. Very few of these EHRs are shared or exchanged easily. Multiple legislative acts and/or regulations (e.g., Social Security Act, Health Maintenance Organizations (HMO) Act, and the Affordable Care Act (ACA), HIPAA, Health Information Technology for Economic and Clinical Health (HITECH) Act, American Health Information Management Association (AHIMA), and/or the like) provide various requirements for storing and maintaining EHRs, such as data formats, InfoSec requirements, Certified Electronic Health Record Technology (CEHRT) criteria, and/or the like. However, accessing or transferring EHRs between HPs 133 usually requires different process and procedures as defined by different HPs 133 and/or as defined by relevant regulations. These repeated EHR processes 135, 137 increase the costs to patients and HPs 133 (in terms of both monetary costs, healthcare resources, and computational resources), and take more time for all persons/entities involved, without providing additional benefits for anybody. There is little or no shared data among various HPs 133. There are also no standards for EHR or related data models. Currently, there are no defined standards for long-term preservation and storage of EHRs, or synchronization of EHRs among multiple HPs 133.

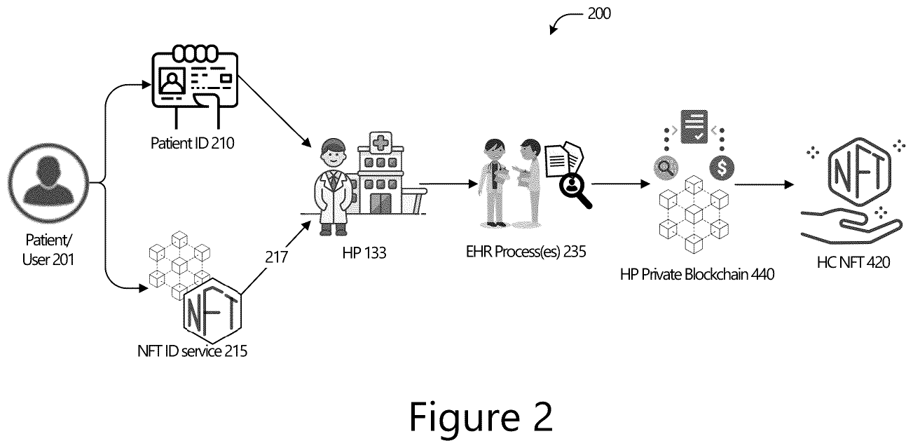

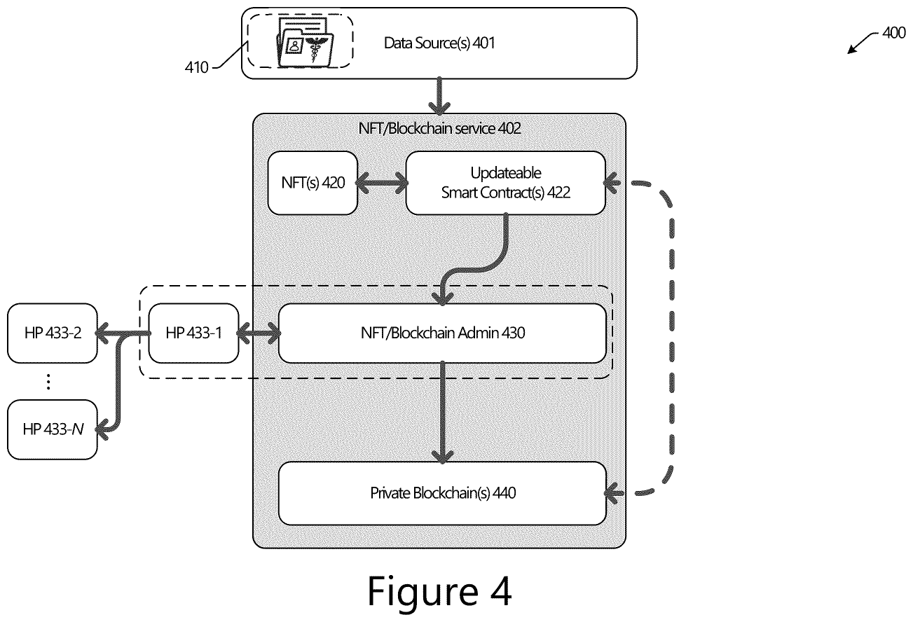

FIG. 2 depicts an example NFT-based EHR ecosystem 200. Here, the user 101 provides their HCD 110 to an HP 133, which performs one or more EHR processes 235 (which may include verification process(es)), and an NFT 420 is created for the verified user 101. The NFT 420 may be generated by an NFT/blockchain service, such as NFT/blockchain service 402 of FIG. 4.

In a first example, the EHR ecosystem 200 does not include an NFT ID system, and the user 101 provides their patient ID 210 (and/or other HCD 110) to the HP 133. The HP 133 performs the EHR process(es) 235 to, inter alia, verify the HCD 101 and/or the user's 101 identity. The verified user 101 and/or individual data items of the verified HCD 110 is/are placed on a private blockchain 440 as respective blocks. The verified user 101 and/or individual data items of the verified HCD 110 is/are also used to create (mint) a healthcare (HC) NFT 420. This example allows the HC NFT 420 to be personalized to the user 101 and the HP 133.

In a second example, the EHR ecosystem 200 can be an “NFT-based EHR ecosystem” that includes an NFT ID service 215 configured to generate a multi-purpose “super ID”. Here, the user 101 provides their patient ID 210 (and/or other HCD 110) to the NFT ID service 215, which generates an NFT ID 217 that is then provided to the HP 133. In some examples, the NFT ID service 215 is/are the same or similar to the NFT system(s) discussed in U.S. Prov. App. No. 63/315,396 filed on 1 Mar. 2022 (“'396”), the contents of which is hereby incorporated by reference in its entirety and for all purposes. Additionally, the NFT ID 217 may be minted in a same or similar manner as discussed in 396. The HP 133 performs none, some, or all tasks/operations of the EHR process(es) 235 using the HCD 101 and/or the NFT ID 217. The HP 133 may perform the EHR process(es) 235 to, inter alia, verify the HCD 101, but may not need to verify the user's 101 identity since that verification has already been performed by the NFT ID service 215. The NFT ID 217 and/or individual data items of the verified HCD 110 is/are placed on the private blockchain 440 as respective blocks. The NFT ID 217 and/or the verified HCD 110 is also used to create (mint) an HC NFT 420 for the user 101.

In the aforementioned examples, the patient ID 210 can be a patient ID card, identification wristband, health insurance card (e.g., US-based insurance card, European health insurance card, and/or the like), Canadian provincial health card, and/or any other identity card and/or other suitable form of identification, such as any of those mentioned herein. Additionally or alternatively, the EHR process(es) 235 may be the same or similar as the processes 135, 137. Additionally or alternatively, the blockchain 440 is private to the HP 133, while in other examples the blockchain 440 is private to the NFT/blockchain service 402 (see e.g., FIG. 4). Additionally or alternatively, the HC NFT 420 can be added to a blockchain 440.

The EHR ecosystem 200 provides a more secure and trustworthy form of EHRs for all healthcare activities, including online and in-person, that can be used at various HPs 133, insurance companies, medical research institutions, and/or other relevant entities and/or organizations (orgs). In some examples, EHRs/HCD 110 can be shared across different healthcare settings, and/or are shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

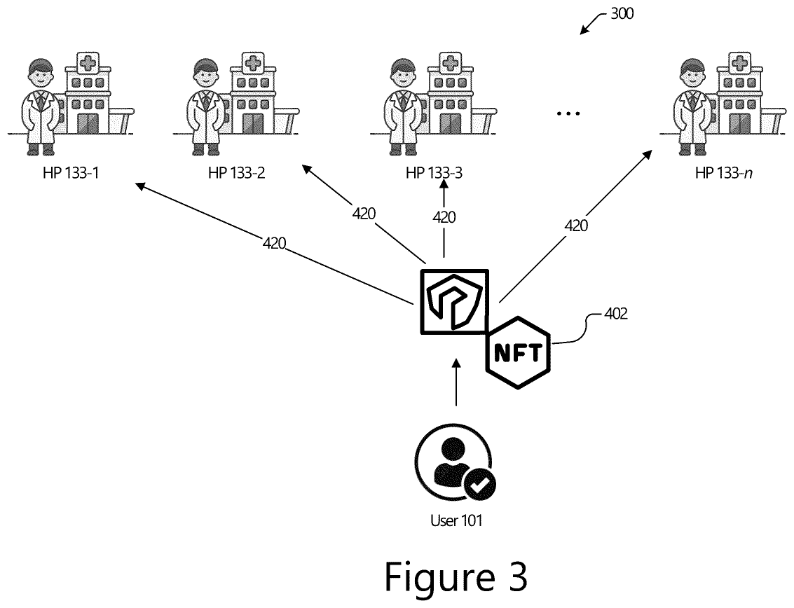

FIG. 3 shows a system 300 in which the NFT-based EHR mechanisms discussed herein can be used at multiple orgs, such as HPs 133-1 to 133-n. Here, a user 101 is able to become verified through the NFT/blockchain service 402, and the verification of the user 101 can be used for multiple HPs 133-1 to 133-n. It should be understood that, although the various examples provided in the present disclosure are described in terms of verifying/validating users 101 for access to EHRs 110 and/or HPs 133, the various embodiments herein are not limited to such examples and can be used for verifying/validating users 101 and/or HCD 110 for other purposes, such as security clearances for employment with government agencies, accessing and/or transferring sensitive and/or confidential information between different entities, and/or the like. The NFT-based EHR mechanisms reduce multiple EHR verification processes 135, 137 to a single EHR verification process 235 at the NFT/blockchain service 402. Additionally, the results of the EHR verification process 235 can be stored in a private blockchain (see e.g., one or more private blockchains 440 in FIG. 4), which can be shared among multiple HPs 133 rather than requiring each HP 133 to perform their own EHR verification processes 135, 137. This provides EHR interoperability among HPs 133 that, for example, belong to separate insurance groups and/or operate within different regulatory regimes and/or geopolitical regions. This also allows users (customers) 101 to own and control their EHR data, such as with personal health records (PHRs), while maintaining EHR data consistency. Furthermore, the EHR verification mechanisms discussed herein can be used to detect irregularities/inconsistencies within the patient's 101 HCD 110, irregular transactions (e.g., electronic exchanges between HPs 133 and/or the like), and/or other potential issues; and raise alarms as required by various regulations.

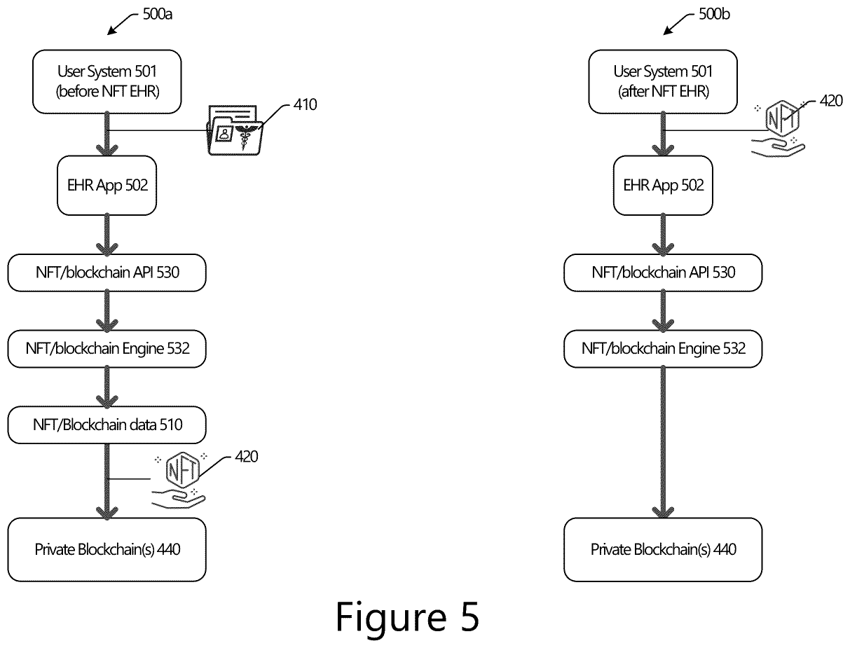

FIG. 4 shows an example NFT-based EHR environment 400. In this example, an NFT/blockchain service 402 obtains user data 410 from one or more data sources 401. The user data 410 is associated with a user (e.g., user/patient 101). The one or more data sources 401 can include a user system operated by the user 101 (e.g., user system 501 of FIG. 5), an HP platform/system 433, a data storage system associated with the HP platform/system 433, or some other third party data source (e.g., government databases, non-governmental org (NGO) databases, and/or the like).

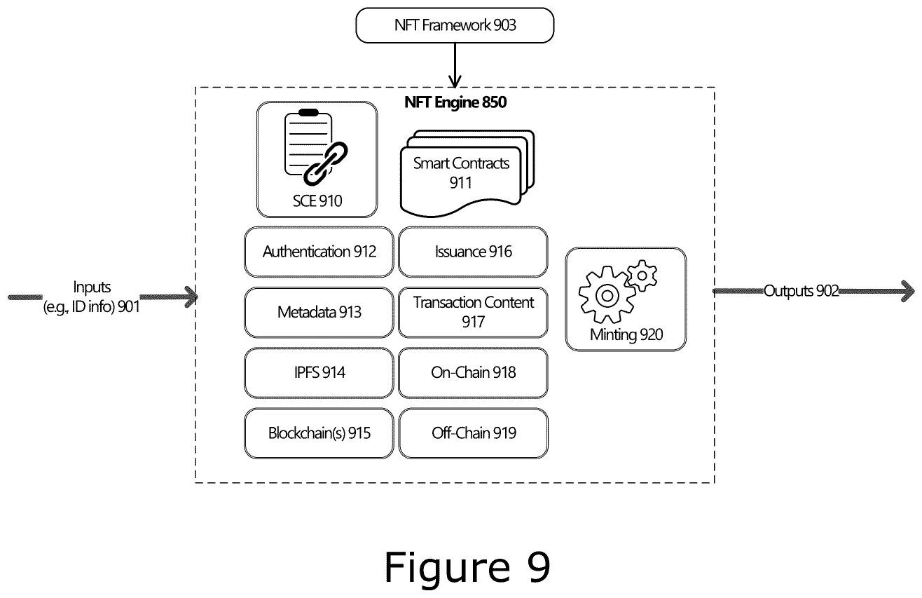

As examples, the user data 410 includes HCD 110, EHR data/information, personal information, confidential information, sensitive information, and/or any other suitable data. Additionally or alternatively, the user data 410 can include physical identity documents (e.g., images and/or videos of physical identity documents), electronic identity documents, authentication or authorization credentials, biometric data, knowledge-based authentication (KBA) data, social media profile data, and/or other types of data, including any types of data mentioned herein. Additionally or alternatively, data related to the user system 501 can be part of the user data 410 or otherwise provided with the user data 410. Examples of the user system 501 data can include location data (e.g., GPS coordinates, time zone information, cellular network location services, WiFi positioning, IP address location correlations, and/or the like), device fingerprint, screen or display resolution, operating system (OS) type and/or version, browser type and/or version, rendering engine type and/or version, device type of the user device 501, device manufacturer, hardware platform type, device serial numbers, system information of the user device 501, and/or other suitable information, data, and/or metadata, including any of the information/data discussed infra with respect to (w.r.t) the inputs 901 of FIG. 9. In some examples, the user data 410 is the same or similar as the HCD 110 discussed previously, and the user system 501 may be the same or similar as the user devices 810, 860, client system 1150, and/or any other suitable user/client device that can be operated by a user, such as user 101 discussed previously. In implementations including an NFT ID system 215 (see e.g., '396), the user data 410 can additionally or alternatively include an NFT ID belonging to a user 101. Some or all of the aforementioned data may be obtained from the user system 501 and/or from third party data sources.

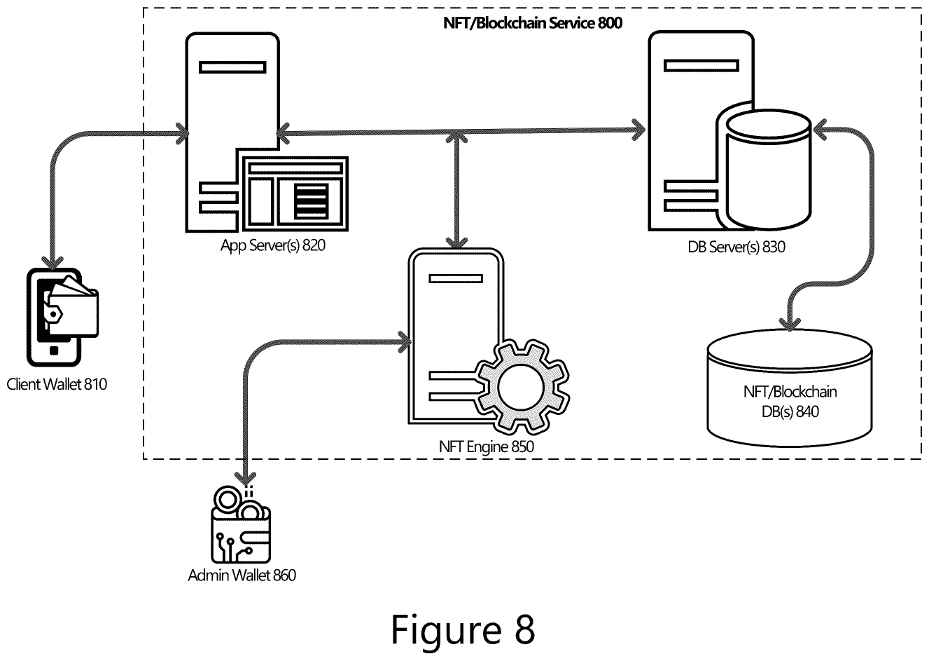

The NFT/blockchain service 402 includes and/or generates NFTs 420 and/or updatable smart contracts 422. The NFT/blockchain service 402 may be the same or similar as the NFT/blockchain service 800 or the NFT engine 850 discussed infra w.r.t FIGS. 8-10. In some implementations, the NFT/blockchain service 402 is a standalone service that is accessible by individual HPs 433. The NFT/blockchain service 402 generates (or mints) NFTs 420 as discussed herein and/or according to, for example, [EIP-721] and/or some other suitable standard(s)/specification(s). The smart contracts 422 can manage multiple token types. For example, a single deployed contract 422 may include any combination of fungible tokens, NFTs, and/or other configurations (e.g. semi-fungible tokens and/or the like). The smart contracts 422 may be the same or similar to the smart contracts 911 of FIG. 9 and/or can be created and/or implemented according to, for example, [EIP-1155], [EIP-2535], and/or the like. In some examples, individual smart contracts 422 can include functions, codes, scripts, and/or the like for performing some or all aspects of EHR process(es) 235.

The NFT/blockchain service 402 also includes a blockchain/NFT administrator (admin) 430, which performs EHR process(es) 235 using an NFT 420 associated with the user of the user system 501. In some examples, the NFT/blockchain service 402 executes one or more smart contracts 422 to perform the EHR process(es) 235. In some examples, based on results of the EHR process(es) 235, the NFT/blockchain service 402 can provide the results, for example, in the form of suitable indicators/notifications to one or more HPs 433. The HPs 433 may be the same or similar as the HPs 133 discussed previously. In this example, the EHR indicators are provided to a first HP 433-1. This information can be shared with, or is otherwise accessible by, one or more other HPs 433-2 to 433-N (where N is a number). Additionally or alternatively, the EHR results/indicators are added to one or more private blockchains 440. In this example, the private blockchains 440 are provided by the NFT/blockchain service 402 (e.g., a Blockchain as a Service (BaaS)); however, in other implementations, some or all of the blockchains 440 can be provided by one or more separate blockchain services.

Additionally or alternatively, individual private blockchains 440 may be provided or maintained for individual HPs 433, for example, where a first private blockchain(s) 440 can be maintained for or otherwise correspond to a HP 433-1, a second private blockchain(s) 440 can be maintained for or otherwise correspond to a second HP 433-2, and so forth up to an Nth private blockchain(s) 440 that can be maintained for or otherwise correspond to an Nth HP 433-N. In these implementations, the set of private blockchains 440 may form a “consortium blockchain”, which is a group of multiple private blockchains 440 that permit sharing of data/blocks amongst themselves for various purposes, such as transparency, accountability, and/or streamlining workflows. While each HP 433 manages their own node or blockchain(s) 440, the data within their blockchain(s) 440 can be accessed, shared, and distributed by different HPs 433 within the consortium. In some implementations, the sharing among the blockchain(s) 440 may be mediated or otherwise controlled by the admin 430.

The private blockchain(s) 440 is/are a type of blockchain and/or blockchain network where only one or a few authorities or orgs have control over the blockchain and/or the blockchain network. By contrast, a public blockchain is permissionless, allows anyone to join, and is usually decentralized. The private blockchain(s) 440 may or may not be decentralized. In some implementations, the admin 430 manages or acts as an administrator of the private blockchain(s) 440, NFT service(s), NFT(s) 420, and/or other entities or services within the NFT/blockchain service 402. Additionally or alternatively, participating HPs 433 can seek approval and consent to join the NFT/blockchain service 402 or otherwise access the permissioned blockchain(s) 440. In some examples, the HPs 433 go through a registration process to join or otherwise access the blockchain(s) 440. In these implementations, HPs 433 participating in the NFT/blockchain service/network 402 can have or obtain knowledge about transactions in the blockchain(s) 440. For example, individual HPs 433 can subscribe to notification services provided by the NFT/blockchain service 402 (or the admin 430) so that they become notified of updates to specified patients' 101 EHRs.

In various implementations, each HP 433 can see their own patient records (EHRs), and multiple HPs 433 can agree amongst themselves if they want to see the other HPs' 433 processes or not. For example, individual private blockchains 440 may be provided or maintained for individual HPs 433, for example, where a first private blockchain 440 corresponds to a first HP 433-1, a second private blockchain 440 corresponds to a second HP 433-2, and so forth up to an Nth private blockchain 440 corresponding to an Nth HP 433-N. In these implementations, the set of private blockchains 440 may form a consortium blockchain (also referred to as a “federated blockchain”), which is a group of blockchains that permit sharing of data/blocks amongst themselves for various purposes, such as transparency, accountability, and/or streamlining workflows. While each org (e.g., HP 433) manages their own node or blockchain 440, the data within their blockchain 440 can be accessed, shared, and distributed by orgs 433 within the consortium. In these implementations, the user data 410 can be shared amongst the HPs 433 as defined by one or more smart contracts 422. In these ways, the NFT/blockchain service 402 reduces multiple EHR processes 135, 137 to a single EHR process 235, and allows the EHR results to be shared with multiple HPs 133. The results are tamper-proof, stored in a private blockchain 440, and in the user's digital wallet in the form of an NFT 420.

The NFT/blockchain service 402 can also enable access to EHRs and allows for EHRs to be updated in real-time (RT) or near RT using suitable APIs, web services, and/or other like interfaces (see e.g., API 530 of FIG. 5). In some implementations, individual HPs 433 configure or otherwise provide the NFT/blockchain service 402 with various database rules, policies, and/or configuration parameters, and the NFT/blockchain service 402 converts the database rules, policies, and/or configuration parameters into a set of RT codes in the form of one or more smart contracts 422, which can then be used to track EHR updates/changes and the like.

The NFT/blockchain service 402 can also detect EHR changes/updates in real-time (RT) or near RT, and can raise alarms and/or issue suitable notifications to relevant entities (e.g., one or more HPs 433, insurance companies, government entities (e.g., regulatory bodies, administrative agencies, and the like), and/or other entities). In some examples, the EHR changes/updates can include changes/updates to existing EHR data, user data 410, and/or HCD 110; newly generated EHR data, user data 410, and/or HCD 110; suspicious, inconsistent, or anomalous transactions; and/or other potential medical record-related errors. In these implementations, the admin 430 converts the EHR policies/rules provided by individual HPs 433 into RT codes in the form of one or more smart contracts 422, which are then used to track various transactions and/or changes to EHR data, user data 410, and/or HCD 110.

Additionally or alternatively, the EHR changes/updates can include predicted diagnostic information. Here, the admin 430 can run the EHR data, user data 410, and/or HCD 110 through one or more AI/ML models (see e.g., '074 and/or '081) to predict potential diagnoses, and/or use AI/ML models provided by individual HPs 433 to detect potential diagnoses. The EHR data, user data 410, and/or HCD 110 can be used as training data to train the AI/ML models, and/or the EHR data, user data 410, and/or HCD 110 can be used as inference data by trained AI/ML models to generate inferences/predictions. In these implementations, the admin 430 converts EHR/diagnostic policies/rules provided by individual HPs 433 into RT codes in the form of one or more smart contracts 422, which are then used to monitor for potential medical issues and/or to track various changes to medical-related issues.

In the aforementioned examples, the RT codes can include conditions, parameters, criteria, functions, methods, API calls, data, and/or the like that can be used for monitoring medical and/or EHR issues and/or transactions related (or potentially related) to the user of the user system 501. In some implementations, the EHR/diagnostic policies and/or rulesets can be defined or otherwise expressed by individual HPs 433 using standardized mechanisms (e.g., language, syntax, schema, and/or the like) using any suitable data format (e.g., JSON, XML, and/or any other data format such as any of those mentioned herein or in '074 and/or '081). In other implementations, the NFT/blockchain service 402 may provide an EHR policy tool (e.g., a web app with a graphical user interface (GUI) and/or the like) that allows individual HPs 433 to define or specify EHR policies and/or rulesets.

Additionally or alternatively, the EHR smart contract(s) 422 are configured to automatically raise flags or issue indicators/notifications when the configured and/or predefined conditions or parameters are met. For example, the admin 430, based on operation of one or more smart contracts 422, can generate notifications, indicators, or alerts based on detected EHR changes, detected diagnostics, and/or detected anomalous/inconsistent transactions. In some implementations, the admin 430 provides such reports/notifications to the individual HPs 433 and/or to specified entities/orgs. Additionally, the monitored data and transactions can be included in one or more blockchains 440, which can be audited or otherwise analyzed by medical professionals, individual patients 101, and/or regulatory bodies. The smart contracts 422 can be configured to send notifications/reports to medical professionals and/or regulatory authorities/agencies on a periodic basis. Additionally or alternatively, authorized personnel can pull desired data in real time from the one or more blockchains 440 via suitable APIs and/or other mechanisms.

In some examples, one or more smart contracts 422 are configured to monitor and enforce privacy and regulation compliance as delineated by various legislation and/or regulations. The NFT/blockchain service 402 enables EHR-related compliance to be updated on a periodic and/or event-based basis, and can be transferred to relevant regulators/authorities at any time. Here, one or more smart contracts 422 can be set up to send compliance records/data to relevant authorities/regulatory bodies on a periodic or event-triggered basis, where the compliance records/data are generated based on data and transactions are stored on the blockchain(s) 440. Additionally or alternatively, the regulators/authorities can pull the data in real time, and in some implementations, the regulators/authorities can define their own smart contract(s) 422 for reporting and/or other compliance purposes.

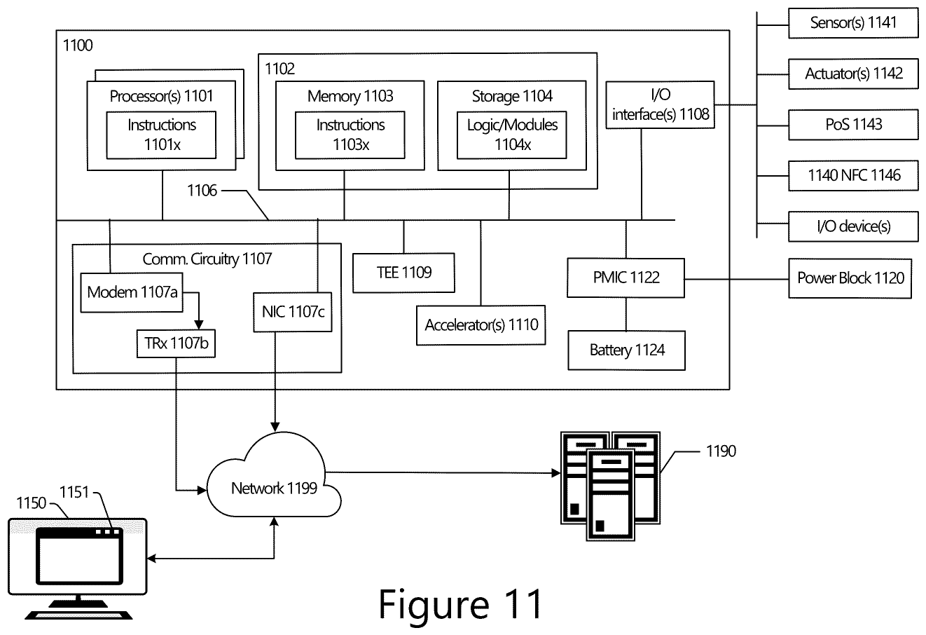

In some implementations, both HPs 433 and EHR NFT holders (e.g., user system 501) belong to the NFT/blockchain service/network 402 and/or have access to the private blockchain(s) 440. The data visibility is layered and controlled by one or more smart contracts 422. The EHR NFT holders (e.g., user system 501) are capable of viewing and/or accessing their user data 410 and approval status. In various implementations, the interactions between the EHR NFT holders (e.g., user system 501) and the blockchain(s) 440 are via a digital wallet (e.g., client/wallet devices 810 of FIG. 8, client app 1151 of FIG. 11, and/or the like) or another suitable app and/or device (e.g., EHR app 502 of FIG. 5, client app 1151 of FIG. 11, and/or the like). Additionally, the smart contracts 522 can be configured with suitable permissions, authorizations, policies, rules, and/or the like to ensure that each HP 433 is only able to access data from other HPs 433 that is/are relevant to its own patients 101. For example, permissions/authorizations may be programmed into the smart contracts 422 so that an HP 433-1 is only able to access data from HP 433-2 that is related to the HP's 433-1 own patients 101 that are also patients 101 of HP 433-2. Additionally or alternatively, each HP 433 can see their own patients' full EHRs, and multiple HPs 433 can agree amongst themselves if they want to see the other HPs' 433 EHRs and/or EHR processes 235 or not. Additionally or alternatively, some or all of the HPs 433 can start with only patients' approval statuses, approval scores, and red flags, if any. Additional data 410 could be shared per one or more smart contracts 422.

The NFT/blockchain service 402 includes very multiuse applications (apps) with one or more microservices and business logic. Examples of the microservices provided by the multiuse apps include one or more of the following: smart contract engine (see e.g., [EIP-1155], [EIP-2535], and the like); authentication; metadata; InterPlanetary File System (IPFS); issuance; transaction; on-chain; off-chain; private blockchain(s) 440; minting (see e.g., [EIP-721], [EIP-5679], and the like).

One example implementation of the NFT/blockchain service 402 includes infrastructure components/elements, client-side components/elements, and administrative (admin) components/elements. The infrastructure elements/components include full stack in the cloud; Linux® OS; Apache®/NGINX server; server-side languages including PHP, Python, JavaScript, and Solidity; and database systems including MySQL, MongoDB, and IPFS. The client-side components/elements include client apps such as a digital/crypto wallet. The client apps may be mobile apps designed for Android®, iOS®, and/or other platforms/OS. The admin 430 components/elements include monitoring services, housekeeping services, and digital/crypto wallet apps (which may be the same or similar as the client-side digital/crypto wallet apps, such as EHR app 502, wallet clients 810, 860, client app 1150, and/or the like). Additionally or alternatively, the NFT/blockchain service 402 can be implemented using a suitable blockchain network/framework, such as, for example, Ethereum (see e.g., [EthereumDoc], the contents of which is hereby incorporated by reference in its entirety and for all purposes) and/or Hyperledger Fabric (see e.g., Hyperledger Fabric documentation, version 2.5 (February 2022), https://hyperledger-fabric.readthedocs.io/en/release-2.5/index.html (“[HyperledgerFabric]”), the contents of which is hereby incorporated by reference in its entirety and for all purposes).

FIG. 5 depicts example NFT EHR procedures 500a and 500b. Procedure 500a takes place before an NFT EHR 420 is minted, and procedure 500b takes place after an NFT EHR 420 is minted.

In procedure 500a, a user (e.g., using system 501) submits user data 410 through an EHR app 502. The EHR app 502 passes the user data 410 to a NFT/blockchain engine 532 via an NFT/blockchain API 530. The NFT/blockchain engine 532 uses the user data 410 to mint or otherwise generate NFT/blockchain data 510. The NFT/blockchain data 510 can include one or more blocks to be verified by one or more blockchain nodes and added to one or more blockchains 440. As examples, the NFT/blockchain data 510 can include individual data items of the user data 410 (e.g., individual data elements/fields of HCD 110, identity documents, individual EHRs, individual user system data items, and/or the like) that are individually hashed by the NFT/blockchain engine 532 using a suitable hash function or other cryptographic mechanism, one or more smart contracts 422, and/or other relevant data. The NFT/blockchain data 510 is used to produce an NFT EHR 420, which is then appended or otherwise added to the blockchain(s) 440. In some examples, the NFT/blockchain data 510 is the NFT EHR 420, or the content of the NFT EHR 420 includes the NFT/blockchain data 510. Additionally or alternatively, the NFT/blockchain data 510 includes a set of blocks for each hashed data item, which are to be added to the blockchain(s) 440.

In procedure 500b, a user (e.g., using a user system 501) submits their NFT EHR 420 through the EHR app 502, and the EHR app 502 passes the NFT EHR 420 to the NFT/blockchain engine 532 via an NFT/blockchain API 530. The NFT/blockchain engine 532 uses the NFT EHR 420 to access and/or decrypt, de-hash, or otherwise obtain the user's EHR in human-readable form. The human-readable EHR data can be obtained from the NFT EHR 420 and/or from one or more transactions recorded in the blockchain(s) 440. Additionally or alternatively, the NFT/blockchain engine 532 can mint or otherwise generate new NFT/blockchain data 510 and/or new NFT EHRs 420 when updates are made to the user's EHR data, which is/are then appended or otherwise added to the blockchain(s) 440.

The NFT EHR procedures 500a and 500b can operate concurrently with one another. In a first example, a first user system 501 performs procedure 500a and a second user system 501 performs procedure 500b. In a second example, a user system 501 generates an NFT EHR 420 via procedure 500a, and then uses that NFT EHR 420 for procedure 500b at some time in the future.

In some examples, the user system 501 is owned/operated by a patient 101 who has possession or access to their EHR data 410, or the user system 501 is owned/operated by an HP agent who submits the EHR data 410 on behalf of the patent 101. In some examples, the EHR app 502 is a patient portal/interface provided by an HP platform 433, a portal/interface provided by the NFT/blockchain service 402, a client app 1151 (e.g., which can be used to access or interact with any of the aforementioned portals/interfaces), a wallet device/app 810, 860 (e.g., which can be used to access or interact with any of the aforementioned portals/interfaces), and/or some other client app and/or device.

The NFT/blockchain engine 532 (also referred to as “blockchain engine 532”, “blockchain node engine 532”, “NFT engine 532”, “engine 532”, and/or the like) is a managed node-hosting service that allows subscribing platforms/services to relay transactions, deploy smart contracts 422, and/or read and/or write blockchain data to/from the blockchain(s) 440 via the NFT/blockchain API 530 (also referred to as “blockchain API 530”, “blockchain interface 530”, “NFT API 530”, “API 530”, and/or the like). Additionally or alternatively, the engine 532 includes the underlying hardware and/or software technologies and infrastructure that powers a blockchain network (e.g., NFT/blockchain service 402) and is responsible for blockchain tasks, such as consensus mechanisms, transaction validation, and block creation. Additionally or alternatively, the engine 532 includes the underlying hardware and/or software technologies and infrastructure that handles NFT-related operations, such as creation (minting), management, transactions (transfers), and displaying NFTs (e.g., NFTs 420).

In some implementations, the engine 532 is or includes an event-driven engine that routes, indexes, aggregates, and/or sequences data to/from the blockchain(s) 440 and various connectors. As examples, the “events” in the event-driven engine can include token/NFT transfer events (e.g., within a single blockchain 440 and/or across multiple blockchains 440), smart contract events (e.g., events issued or detected by one or more smart contracts 422), correlated on-chain and/or off-chain events, and/or the like. As examples, the NFT/blockchain engine 532 can be an off-chain database engine, analytics engine, orchestration engine (e.g., FireFly Core Orchestration engine as discussed in Hyperledger FireFly v1.1.2 (12 Sep. 2022), https://hyperledger.github.io/firefly/v1.1.2/(“[FireFly]”), the contents of which is hereby incorporated by reference in its entirety and for all purposes), a BaaS platform, a consortium manager, a rules engine, and/or the like.

Additionally or alternatively, the NFT/blockchain engine 532 is or includes a consortium engine 532, the NFT/blockchain API 530 is a consortium API 530, and the blockchain(s) 440 form or are otherwise part of a consensus blockchain 440. In these implementations, the engine 532 can be a consortium manager/orchestrator that determines and/or selects the rules engine(s) and the smart contract(s) 422 to be used in a blockchain 440 (or blockchain consortium) to address transactional requirements and/or for other purposes. Additionally or alternatively, the engine 532 can be, include, or otherwise access a rules engine that defines the set of rules, policies, and/or configurations to be used for transaction handling and/or the business logic to be used for satisfying the blockchain execution workflow.

Additionally or alternatively, the NFT/blockchain engine 532 is, includes, or otherwise is capable of accessing one or more blockchain connectors (see e.g., Xu et al, The Blockchain as a Software Connector, IEEE, 13TH WORKING IEEE/IFIP CONFERENCE ON SOFTWARE ARCHITECTURE (WICSA), pp. 182-191 (5 Apr. 2016), [FireFly], and/or Belchior et al., A survey on Blockchain Interoperability: Past, Present, and Future Trends, arXiv: 2005.14282v3 [cs.DC] (22 Mar. 2021), the contents of each of which are hereby incorporated by reference in their entireties and for all purposes). In any of the example implementations discussed herein, the engine 532 and/or API 530 are provided by, or otherwise part of the NFT/blockchain service 402. Additionally or alternatively, the API 530 can be an open source API, web service, IPC, middleware, software connector(s), and/or other interface or connection mechanism.

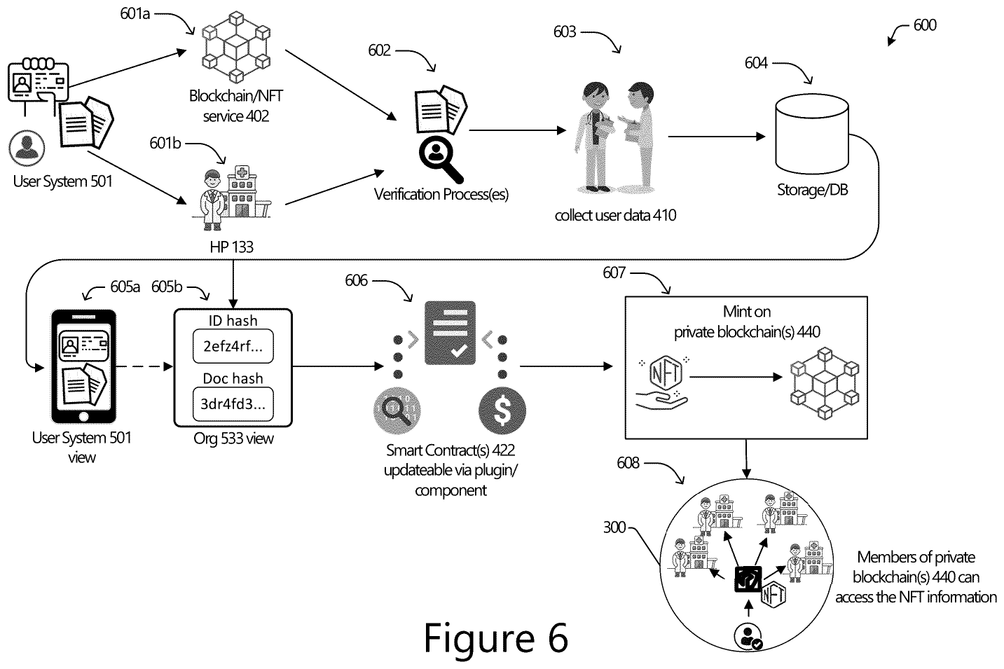

FIG. 6 depicts an example NFT EHR procedure 600. Procedure 600 begins where a customer of an HP 433 (e.g., user system 501) visits or accesses the NFT/blockchain service 402 (e.g., using a suitable website, application (app), web app, or other suitable interface provided by the NFT/blockchain service 402) (601a) and/or accesses the HP's 433 platform (e.g., using a suitable website, application (app), web app, or other suitable interface provided by the HP 433) (601b). Here, the user system 501 provides user data 410 to the NFT/blockchain service 402 (601a) and/or to the HP's 433 platform (601b). In examples where an NFT ID is provided as part of the user data 410, the NFT/blockchain service 402 and/or the HP's 433 platform will retrieve or otherwise determine relevant identity information from the NFT ID 420.

Next, the NFT/blockchain service 402 and/or the HP 433 platform performs verification process(es) to verify and/or validate the user data 410 supplied by the user system 501 (602), and collects (additional) user data 410 from the user system 501 and/or from other data sources (603). In some examples, the verification process(es) (602) and the data collection processes (603) are part of, or correspond to, the EHR process(es) 235 discussed previously. The verified/validated user data 410 is then stored in a suitable storage system or database (DB) system (604). In some examples, the user data 410 is hashed and stored on one or more private blockchains 440 (604) as its own block (e.g., as an NFT or a “EHR block”). Additionally or alternatively, each platform/system (e.g., the NFT/blockchain service 402 and the HP 433 platform) hashes the user data 410 separately and stores the hashes on the private blockchain(s) 440 (604). In some examples, each piece of verified/collected user data 410 is hashed separately from other pieces of user data 410 (e.g., each provided identity document, photographs/video data, each piece of personal information, individual EHRs, KBA data, and so forth may be hashed individually), and stored as respective blocks on the blockchain(s) 440. Additionally or alternatively, some or all user data 410 can be combined or otherwise hashed together and stored as a single block on the blockchain(s) 440. Additionally or alternatively, in examples where the user data 410 includes physical documents and/or identity documents (e.g., electronic identity documents and/or scans of physical identity documents), the physical and/or identity documents themselves are not placed on the blockchain(s) 440; instead, copies (or electronic versions) of the physical and/or identity documents can be stored in encrypted format in private servers and/or data storage systems owned/operated by the NFT/blockchain service 402 (see e.g., servers 820, 830, 850 and/or NFT/blockchain storage 840 of FIG. 8) and/or private servers owned/operated by the HP 433 platform (604).

Next, an EHR block and/or NFT 420 that contains the one or multiple hashes is sent to the wallet app on the user system 501 (605a) and/or the HP platform 433 (605b). The EHR block and/or NFT 420 is also sent to the user's smart contract 422 with the HP 433 (606). In this example, the smart contract 422 is updateable via a suitable plugin or other component. The smart contract 422 and/or the EHR block/NFT 420 are minted on the private blockchain(s) 440 (607). After minting, the EHR block/NFT 420 is accessible by members of the private blockchain(s) 440, such as one or more other HPs 433 (608).

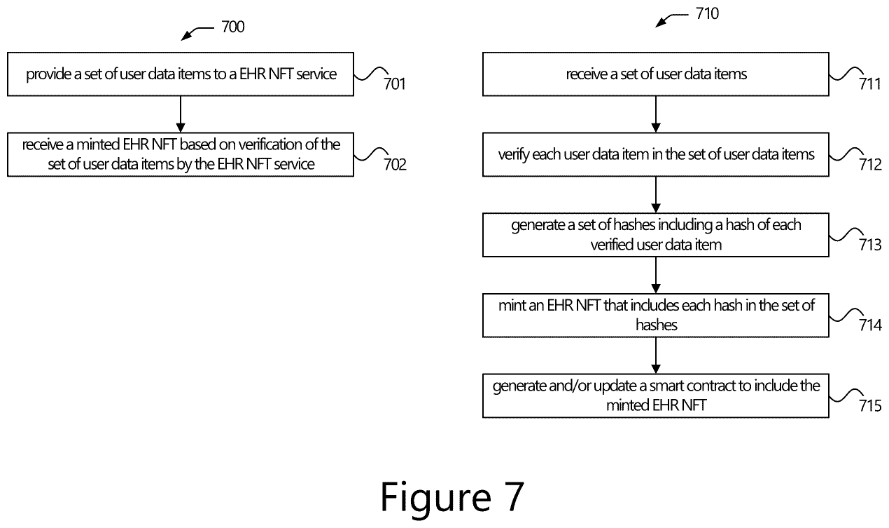

FIG. 7 depicts example processes 700 and 710 for practicing various EHR NFT aspects discussed herein. The example operations of processes 700 and 710 can be arranged in different orders than shown, one or more of the depicted operations may be combined and/or divided/split into multiple operations, depicted operations may be omitted, and/or additional or alternative operations may be included in any of the depicted processes.

Process 700 may be performed by a user device (e.g., users/client devices 101, 501, 810, 860, 1001, 1150) for obtaining EHR NFT services. Process 700 includes: at operation 701, providing a set of user data items (e.g., EHR data 101, user data 410, and/or inputs 901) to an EHR NFT service (e.g., NFT/Blockchain service 402, 800); and at operation 702, receiving, from the EHR NFT service, a minted EHR NFT (e.g., NFT 902) based on verification of the set of user data items by the EHR NFT service. In some examples, the set of user data items (or a subset of user data items) are provided to the EHR NFT service via a user app (e.g., wallet app 810 and/or app 1150), and the minted EHR-NFT is received from the EHR NFT service via the user app (e.g., wallet app 810 and/or app 1150). Additionally or alternatively, the set of user data items (or a subset of user data items) are provided to the EHR NFT service via an HP platform 133, 433, and the minted EHR-NFT is received from the EHR NFT service via the HP platform 133, 433.

Process 710 may be performed by a EHR NFT service (e.g., NFT/Blockchain service 402, 800) for providing EHR NFT services to HPs 133 and/or users 101. Process 710 includes: at operation 711, receiving a set of user data items (e.g., EHR data 101, user data 410, and/or inputs 901) associated with a user (e.g., users/client devices 101, 501, 810, 860, 1001, 1150); at operation 712, verifying each user data item in the set of user data items; at operation 713, generating a set of hashes, wherein each hash in the set of hashes corresponds to a verified user data item in the set of user data items; at operation 714, minting a EHR NFT (e.g., NFT 902) that includes each hash in the set of hashes; and at operation 715, generating and/or updating a smart contract to include the minted EHR NFT (e.g., NFT 902).

FIG. 8 depicts various elements/components of the NFT/blockchain service 800. In this example, the NFT/blockchain service 800 includes an NFT engine 850 communicatively coupled with one or more app servers 820 and one or more database (DB) servers 830. The server(s) 820, 830, and/or 850 operate distributed applications to provide the NFT/blockchain service 800 to client/wallet devices 810. The server(s) 820, 830, and/or 850 may be located in one or more data centers (e.g., provided by a cloud computing service or the like), at the network's “edge”, and/or in some other arrangement or configuration. One or more of the servers 820, 830, and/or 850 may be virtual machines (VMs) or other isolated user-space instances provided by a cloud computing service, edge computing service, and/or the like. Furthermore, some or all of the server(s) 820, 830, and/or 850 may also provide various administration capabilities to support the various aspects discussed herein. In various implementations, the servers 820, 830, and/or 850 can be located at different geographic locations such as, for example, in different data centers, co-located with different network access nodes, and/or the like. In one example implementation, the infrastructure may include a full stack in the cloud, the servers 820, 830, and 850 implementing a suitable Linux distribution (distro), operating NGINX and/or Apache® web/app servers, where the server-side languages of the distributed apps are written using PHP, Python, JavaScript, and Solidity, and the DB systems (e.g., NFT DBs 840) are implemented using MySQL, MongoDB, and IPFS.

In some implementations, the server(s) 820 receive user data 410 as inputs (e.g., inputs 901 in FIG. 9) via a front-end portal (e.g., mobile app, web app, and/or website, not shown). As examples, the user data 410 includes physical or electronic identity documents and/or other information such as contact information, authentication credentials, biometric data, credit report scores and/or other credit report data, EHR data, knowledge-based authentication (KBA) data, and/or the like. In some implementations, physical identity documents and/or other physical media can be scanned in and uploaded by individual users (e.g., using their client/wallet device/apps 810 or 860) to the server(s) 820 using a suitable user interface provided by the front-end portal. This user interface can also be used to provide (upload) electronic documents/information to the server(s) 820. Additionally or alternatively, the same or different interfaces can be used to supply the EHR data and/or KBA data. Additionally or alternatively, the user data 410 can be provided to the server(s) 820 via third party web/mobile apps and/or Web2 apps using suitable APIs and the like. Examples of such APIs include remote APIs and/or web APIs (e.g., Representational State Transfer (REST or RESTful) API, HTTP/2, Simple Object Access Protocol (SOAP) API, salesforce.com Apex API, and/or the like), a web services (WS) (e.g., Apache® Axi2.4 or Axi3, Apache® CXF, a JSON-Remote Procedure Call (RPC) API (e.g., Ethereum JSON-RPC API implemented by a public or enterprise Ethereum® blockchain platform), JSON-Web Service Protocol (WSP), Web Services Description Language (WSDL), XML Interface for Network Services (XINS), Web Services Conversation Language (WSCL), Web Services Flow Language (WSFL), RESTful web services, Flow Wallet API, and/or the like), and/or some other suitable API and/or WS. Additionally or alternatively, the APIs can include IPCs such as, for example, sockets, message queues, anonymous pipes, named pipes, shared memory, message passing, memory-mapped files, message-oriented middleware, Thrift, Common Object Request Broker Architecture (CORBA), Electron asynchronous IPC, and/or other IPC or related technologies. The terms “users 810, 860”, “individuals 810, 860”, “wallet apps 810, 860”, “wallet devices 810, 860”, “client systems 810, 860”, “client devices 810, 860”, and/or the like may be used interchangeably throughout the present disclosure, and these terms may refer to the devices/apps 810, 860 themselves and/or the users of the devices/apps 810, 860, unless the context dictates otherwise. Furthermore, the term “users 810, 860” may refer to a “user 810”, a “user 860”, or both a “user 810 and user 860”. Additionally or alternatively, the users 810, 860 may be the same or similar to the user 101 and/or user system 501 discussed previously.

Web 2.0 platforms (“Web2”) are websites and/or apps that emphasize user-generated content, ease of use, participatory culture and interoperability (i.e., compatibility with other products, systems, and devices) for end users (e.g., users 810, 860). A Web2 platform allows users (e.g., users 810, 860) to interact and collaborate with each other through, for example, social media dialogue as creators of user-generated content in a virtual community. Data and content are centralized in a relatively small group of companies sometimes referred to as “Big Tech”. Examples of Web2 platforms include social networking sites or social media platforms (e.g., Facebook®, Instagram®, Twitter®, and the like); blogs, wikis, and folksonomies (“tagging” keywords on websites and links); video sharing platforms (e.g., YouTube®, Vimeo®, TikTok®, and the like); image sharing platforms (e.g., Flickr®, Imgur®, and the like); hosted services; Web apps and mobile apps; collaborative consumption platforms; and mashup apps.

Web 3.0 platforms (“Web3”) represent a new iteration of the World Wide Web based upon blockchain technologies, which incorporates concepts including decentralization and token-based economics. Web3 platforms usually revolve around the idea of decentralization and often incorporate blockchain technologies, such as various cryptocurrencies and NFTs.

The received user data 410 may be provided to the DB servers 830, which may store the data in the NFT DB(s) 840 for temporary and/or persistent storage. The DB(s) 840 can be blockchains, distributed ledgers, and/or any suitable relational and/or non-relational DB(s) stored by one or more suitable data storage devices. The DB servers 830 may also be configurable to destroy the stored information within a predefined or configurable period of time (e.g., by deleting such information from the DB(s) 840). In some implementations, the servers 820, 830, and/or 850 can also be configurable or operable to generate reports and statistics to authorized recipients upon request. In embodiments, the received user data 410 is provided to the NFT engine 850 for generation of suitable NFTs according to the various embodiments discussed herein.

The NFT engine 850 is an adaptable and multipurpose software and/or hardware element that generates an NFT-based form of digitized/tokenized IDs, using blockchain technology, smart contracts, distributed ledgers, digital wallets 810, distributed apps, and/or identity data (e.g., identity documents, personal information, biometric data, and/or the like). In some implementations, the NFT engine 850 is an application operated by one or more computing devices, such as one or more servers (e.g., a cluster of cloud compute nodes, one or more edge compute nodes, network functions and/or application functions in a cellular network, and/or the like). Additionally or alternatively, the NFT engine 850 may be a distributed application operated by multiple app servers 820 or other devices.

The NFT engine 850 accepts inputs (e.g., inputs 901 in FIG. 9) from any number of sources, processes the inputs, and produces NFTs (e.g., outputs 902 in FIG. 9) that can be used on Web2 and/or Web3 platforms, and/or carried by individuals via digital wallets 810. The NFT engine 850 can take any form of physical ID, digital ID, EHR data, KBA data, and/or other information, process them in complex ways as defined by the users (e.g., users 810, 860), and generate and output an NFT form to be used in various applications (apps) such as Web2 and/or Web3 apps. The generated NFTs are then stored in the NFT DB(s) 840 via the DB servers 830 in one or more distributed ledgers or blockchains (e.g., blockchains 915 in FIG. 9).

Blockchain is a technology that uses cryptographic mechanism(s) to create secure linkages between records (referred to as “blocks”). Additionally or alternatively, a blockchain (e.g., blockchain 915 in FIG. 9) is a type of DB, which may or may not be a distributed DB, that can record transactions in a public or private peer-to-peer (p2p) network. For purposes of the present disclosure, the term “blockchain” can refer to a blockchain DB and/or the technologies used to create, maintain, and/or modify a blockchain DB. In the context of blockchain technologies, a “ledger” refers to a series of interconnected records or blocks. Blockchain technologies distribute a DB of transactions (a ledger) to some or all participants in a network. The participants in a blockchain network are referred to as “peers” or “nodes”. In some examples, the client devices 501, 810, 860, servers 820, 830, 850, individual wallet apps, and/or other apps and/or compute nodes, including any of those discussed herein, can operate as nodes or peers in a blockchain network. In some implementations, some or all nodes in a blockchain network can include or otherwise implement the same or similar cryptographic mechanism(s) and/or blockchain service(s) 402 discussed herein. An individual blockchain (or ledger) is shared, replicated, and synchronized among one or more nodes in the blockchain network.

In some implementations, a blockchain has no central administrator or centralized data storage system, however, in other implementations a centralized system may be used to store and maintain individual blockchains, validate blocks, and/or perform other functions. Most ledgers are used to track a specific type of information or transactions, such as cryptocurrency, securities, tokens, NFTs (including NFT IDs, EHR NFTs, and/or the like), smart contracts, and/or the like; however, some ledges may be used to track multiple types of information/transactions.

As mentioned previously, blocks in a blockchain are linked together using some identifier or other like mechanism. Each block (record) in a blockchain includes, for example, transaction data, a timestamp, and a block ID. Typically, blocks are linked together using cryptographic mechanism(s) where each block contains a cryptographic hash of a previous block. In some implementations, the hash of the previous block may act as the block ID for a block, or a hash of the block itself (including the hash of the previous block) may act as a block ID for a block. In some implementations, the blocks may be encrypted to enhance security. The timestamp proves that the transaction data existed when the block was published in order to get into its hash. Each block contains information about the block previous to it, forming a chain, with each additional block reinforcing the ones before it. This makes blockchains resistant to modification of their data because once recorded, the data in any given block cannot be altered retroactively without altering all subsequent blocks. An advantage of using blockchain technology is that transactions performed on a blockchain are immutable, which means that the transactions cannot be changed or altered without permission from the network. This creates an accurate and nearly unchangeable record (or chain of records) that can be used to verify each transaction, such as each transfer of title or ownership, identity changes and/or changes/updates to identifying information, and the like.

The nature of the cryptographic linkage makes the series of interconnected records more resistant to spontaneous changes to data in a record because publishing an update to an individual record entry also requires altering the cryptographic hash that was generated as the record was created, and any records added to the ledger after an altered record must be re-validated and re-added to the ledger (also with updated hashes). In a blockchain, a change to a record's value is typically published as a new ledger entry. When this single blockchain is connected to some kind of network that can handle communication between nodes and provide a system for one or more nodes to verify changes to network data, then an individual blockchain can be replicated across different nodes in the network. This replication creates multiple blockchain ledgers, containing identical records that have been independently verified. In these ways, a blockchain is provides immutability for the transactions that are tracked. In some implementations, a node pipeline may be used to verify blockchain transactions as discussed in [Flow], [Flow-NFT], and/or the like).

Additional or alternative examples of information that can be included in a block include: a timestamp (e.g., the time when the block was minted and/or mined or created), a block number (e.g., the length of the blockchain in number of blocks), fee per gas or gas price (e.g., the minimum fee per gas required for a transaction to be included in the block), difficulty (e.g., the effort required to mint and/or mine the block), mix hash (e.g., a unique ID for the block), parent hash (e.g., a unique ID for the block that came before (i.e., the previous block or the top-most block)), transaction data (e.g., the transaction included in the block (e.g., inputs 901 in FIG. 9)), state root (e.g., the entire state of the system including, for example, account balances, contract storage, contract code, account nonces, identity documents and/or identity data, HCD 110 (or user data 410), and/or the like), and a nonce (e.g., a hash that, when combined with the mix hash, proves that the block has gone through a consensus (e.g., proof-of-work, proof-of-stake, and/or the like) and/or other suitable verification/validation process(es)). Additional or alternative information may be included in a block in other implementations.

Blockchains can be managed by a p2p network for use as a publicly distributed ledger, where nodes collectively adhere to a protocol to communicate and validate new blocks. These blockchains are referred to as “public blockchains”. Public blockchains are permission-less by nature, allowing most users to join, and are usually decentralized (e.g., stored and updated by multiple compute nodes). By contrast, a private blockchain is a blockchain network where only one or a few authorities or organizations have control over the blockchain network including, for example, rules and/or policies for adding new blocks to the blockchain and the like.

A non-fungible token (NFT) is a unique and non-interchangeable unit of data stored on a blockchain. NFTs use a digital ledger to provide a public certificate of authenticity or proof of ownership, but do not restrict the sharing or copying of the underlying digital files. The lack of interchangeability (fungibility) distinguishes NFTs from blockchain cryptocurrencies, such as Bitcoin, Ethereum, and the like. The mapping from original data to a token uses methods which render tokens infeasible to reverse in the absence of the tokenization system, for example, using tokens created from random numbers. Tokenization systems provide data processing applications with the authority and interfaces to request tokens, or detokenize back to sensitive data. NFTs can be associated with reproducible digital files such as photos, videos, and audio. In various implementations, the NFTs may be generated in accordance with suitable standards, such as [EIP-20], [EIP-721], [EIP-1155], [EIP-3525], [EIP-5528], [EIP-5679], [EIP-5727], [Flow-NFT], and/or the like.

As mentioned previously, the NFTs generated by the NFT engine 850 can be stored in a client wallet 810 (also referred to as a “digital wallet 810”, “cryptocurrency wallet 810”, “wallet device 810”, “wallet 810”, or the like). The wallet 810 is a device, physical medium, program, or a service that stores a user's credentials (cryptographic private keys and/or public keys, digital certificates, and the like) for completing transactions such as cryptocurrency and/or other blockchain-related transactions. In some implementations, the wallet 810 may be a software element (e.g., mobile payment app) such as Apple Pay®, Google Pay®, PayPal®, Venmo®, and/or other like application operated by a user device.

The user device may be a mobile device such as a laptop, smartphone, tablet, wearable (e.g., smartwatch), and/or the like, or any other suitable user device such as those discussed herein. In these implementations, the wallet 810 may be a platform-specific app (e.g., iOS app, Android app, and the like). In other implementations, the wallet 810 may be a standalone hardware wallet device such as the Ledger Nano X provided by Ledger SAS®, the YubiHSM2 provided by Yubico®, the Trezor Model T® provided by SatoshiLabs S.R.O., and/or the like. The wallet 810 may be a “hot wallet” (e.g., a wallet that stores the user credentials, a network-connected application, and the like) or a “cold wallet (e.g., a wallet that stores the user credentials offline and/or disconnected from the internet or other network).

In various implementations, the user credentials are associated with a state of a user's account in a blockchain-based framework. The wallet 810 allows the user to make transactions, where the public key of the public/private key pair allows other wallets to, for example, make payments to the wallet 810 (e.g., using the wallet's 810 network address, app/wallet identifier, or the like) and/or mint tokens and/or NFTs, and the private key of the public/private key pair allows the wallet 810 spend currency or cryptocurrency stored by the wallet 810 and/or in the blockchain as well as transfer tokens and/or NFTs to/from other wallets. In addition to this basic function of storing the keys, the wallet 810 also offers the functionality of encrypting and/or digitally signing information or electronic documents. Signing can for example result in executing a smart contract, a cryptocurrency transaction, identification process, and/or legally signing a document.

Additionally, an administrator (admin) that operates the admin wallet 860 (also referred to as “admin 860”) associated with an org/entity defines the requirements and functions of each NFT type. In some implementations, the admin 860 is involved in the creation of the NFTs by coding, compiling, deploying, and running a smart contract in/at the NFT engine 850. Additionally or alternatively, the admin 860 gathers information provided by individual users, encrypts the information, and then uploads the information to an InterPlanetary File System (IPFS) (see e.g., IPFS 914 in FIG. 9 and [IPFS]) and/or a private data storage server. The admin 860 uses a wallet 860 which may be the same or similar as the wallet 810. In addition to the aspects discussed previously w.r.t the wallet 810, the wallet 860 may also perform monitoring and/or housekeeping functionality.

Furthermore, the wallets 810, 860 implement a wallet interface with the NFT/blockchain service 800 in order to provide inputs (e.g., inputs 901 in FIG. 9) to the NFT/blockchain service 800, and to accept outputs (e.g., NFTs/outputs 902 in FIG. 9) such as accepting transfers and the like. This wallet interface may be the aforementioned NFT front-end portal or some other interface.

FIG. 9 depicts an example implementation of the NFT engine 850. The NFT engine 850 is or includes one or more multi-use applications that include multiple microservices and business logic. In this example, the microservices of the NFT engine 850 include a smart contracts engine (SCE) 910, one or more smart contracts 911, authentication engine 912, metadata 913, IPFS microservice 914, one or more blockchains 915, issuance microservice 916, transaction content 917, on-chain microservice 918, off-chain microservice 919, and minting engine 920.

As alluded to previously, the NFT engine 850 accepts various inputs 901 (e.g., the user data 410 discussed herein), performs various operations (e.g., using the microservices 911-920), and produces NFT(s) as outputs 902 (also referred to herein as “NFTs 902” or the like). The inputs 901 may include various identity documents (e.g., electronic identity documents and/or physical identity documents scanned in or otherwise transformed into electronic form) and/or other data associated with a particular individual or user.

Additionally or alternatively, the inputs 901 may include user-supplied/provided personal data such as, for example, name, physical home address, physical employment address, phone number, email address, medical data (e.g., lab test results, vaccination records, EHRs, and/or the like), and/or any other types of data. Additionally or alternatively, the inputs 901 may include other information related to a user such as, for example, rental agreements, mortgage statements, utility bills, cell phone service bills, and/or other financial instruments and/or financial institution documents. Additionally or alternatively, the web/app interface may request other information to be provided by the user such as biometric data and/or the like.

Additionally or alternatively, the inputs 901 may include other information collected from the user device such as any type or combination of network addresses, timestamp, geolocation information associated with the user's access of the NFT/blockchain service 800, user ID (userId), client app ID, client app type (e.g., browser, wallet/payment app, or the like) and/or version, an app session ID, user agent string, operating system (OS) type and/or version, app and/or OS vendor, a network address (such as those discussed herein), a network session ID, a device ID or serial number, a product ID, EPC, RFID tag ID, an integer assigned to the user 810, 860 by a Unix-like OS (e.g., effective user ID (euid), file system user ID (fsuid), saved user id (suid), real user id (ruid), etc.), a cookie ID, a realm name, domain name/ID, logon user name or credentials, network credentials, social media account name, session ID, device fingerprint of a user/wallet device 810, 860, a digital certificate, and/or any other like ID associated with a particular user 810, 860 or user/wallet devices 810, 860) and/or the like. This information may be collected by program code/script embedded in webpages/web apps of the NFT front-end portal, which when executed by the user device, causes the user device to collect such data and send it to the NFT/blockchain service 800 as inputs 901. Additionally or alternatively, sensitive data and/or confidential information may be collected. The personal, sensitive, and/or confidential data can be anonymized and/or pseudonymized or otherwise de-identified using suitable anonymization/pseudonymization technique(s).

The particular types of inputs 901 used may be specified or configured by a particular platform, agency, org, HP 133, and/or other entity. In some implementations, individual users 810, 860 provide the inputs 901 to the NFT/blockchain service 800 (e.g., using their client/user devices). In other implementations, an admin 860 collects the information and provides the information as inputs 901 to the NFT/blockchain service 800 (e.g., using their client/user devices and/or a specialized/secure admin portal).

In addition to or alternatively to identity documents, other user data 410 may be provided as inputs 901, such as, for example, user generated text, images, video, audio content, HCD 110 and/or user data 410, KBA data, and/or other data. In some implementations, biometric analysis and/or processing may be incorporated into the NFT generation process (e.g., including biometrics being provided as inputs 901). The biometric inputs 901 may include physiological biometrics (e.g., fingerprints, face/facial features, DNA, palm print, body part geometry, vein patterns, eye features, odor/scent, voice features or voiceprint, neural oscillations and/or brainwaves, pulse, electrocardiogram (ECG), pulse oximetry, and/or the like) and/or behavioral biometrics (or “behaviometrics”) (e.g., typing rhythm, gait, signature, behavioral profile features, voice features, and/or the like). In these implementations, the NFT engine 850 accepts one or more biometrics as inputs 901, which are then combined together (and/or with other inputs 901) and hashed. The hash will then become part of a smart contract (e.g., as generated and/or operated by the smart contracts engine 911) to generate NFTs where use of biometrics is desired (e.g., identify-proofed NFTs, NGO/commercial NFTs, and/or social NFTs).

The authentication engine 912 is a microservice that authenticates individual users 810, 860 that submit or otherwise provide inputs 901 to the NFT engine 850. In some implementations, individuals 810, 860 register or enroll with the NFT/blockchain service 800 by providing user data 410 (e.g., as inputs 901) to the NFT/blockchain service 800 using a suitable user interface operated by a user system (e.g., wallet devices 810, 860). For example, a web/app interface may be provided to the user system and/or wallet devices 810, 860 to access a web/app server 820 to provide the information to the NFT engine 850, which is then stored by the DB server(s) 830 in NFT DB 840. This web/app interface may include form fields for users 810, 860 to enter the information and/or other PII. Additionally or alternatively, client-side script, tags, program code, and the like may be included in the NFT front-end portal/interface that collects some of the user data 410/inputs 901 from the user 810, 860 when accessed by the user/wallet devices 810, 860. The information used for authentication may be the same or similar to the user data 410/inputs 901 used to create the NFTs 902 and/or may be a subset of the user data 410/inputs 901. In some implementations, the authentication engine 912 may verify the user data 410/inputs 901 against authoritative sources (e.g., credit bureaus, government database(s), corporate database(s), and the like). Additionally or alternatively, the authentication engine 912 authenticates a user's 810, 860 identity using authentication or authorization credentials, biometric data, EHR data, and/or KBA data. The authentication engine 912 also provides access control to restrict what accounts can mint items, including defining and enforcing ownership and role-based access schemes (see e.g., [OZContracts]). Additionally or alternatively, the authentication engine 912 authenticates a user's 810, 860 identity using third party identity verification services, in which case the authentication engine 912 can communicate with the third party identity verification service via suitable APIs and the like.

The NFTs (e.g., NFT(s) 420, 902) and/or other tokens may be created (or “minted” in the parlance of the NFT arts) by the minting engine 920, which is discussed in more detail infra. The minting process may be based on one or more of the smart contracts 911, which are generated by the SCE 910 based on configurations and/or templates provided by admins 860. The SCE 910 may implement any suitable smart contract mechanism (e.g., [Solidity], [EEA-CS7], and/or the like) to generate smart contracts 911. In some implementations, a suitable integrated development environment (IDE) or software development environment may be used to define and generate the configurations used by the SCE 910 to generate and deploy the smart contracts 911. The SCE 910 combines one or more inputs 901 and hashes the combined inputs 901. The hash will then become part of a smart contract 911 (e.g., as generated and/or operated by the SCE 911), which are then executed by the minting engine 920 to generate NFTs 902.

The NFT engine 850 can include or provide runtime environment(s) in which smart contracts 911 can be executed. Smart contracts 911 are reusable snippets of code (e.g., a program, application, set of trigger functions, or transaction protocol) that is intended to automatically execute, control, or document relevant events and actions according to some predefined criteria or conditions. Additionally or alternatively, smart contracts 911 are computer programs, scripts, and/or code snippets that are executed by nodes in a blockchain network to digitally facilitate, verify, and/or enforce the negotiation or performance of a contract, which may be made partially or fully self-executing and/or self-enforcing. Smart contracts 911 can implement a contract between two or more parties, such as where the execution is guaranteed and auditable. In some examples, the smart contracts 911 include codes, real-time codes, scripts, programs, API calls, and/or the like that verify, validate, and/or authenticate one or more inputs 901 (e.g., HCD 110, 410, and/or the like), monitor for EHR updates/changes, and/or monitor EHR and/or healthcare-related transactions, and/or for AI/ML model training/inference aspects.

Each smart contract 911 includes a set of policies, rules, configurations, functions, and/or data that govern what happens whenever an interaction with an NFT 902 takes place. In some implementations, each NFT 902 is associated with a smart contract 911, which includes a set of functions of the NFT 902 that allow applications to interact with the NFT 902. Additionally or alternatively, smart contracts 911 include one or more functions that take relevant or desired data (e.g., unique IDs, content items (e.g., HCD 110), name, inheritance (if applicable), metadata, and/or any other relevant data structures, variables, parameters, and/or the like) as arguments and assigns it/them to appropriate variables. Smart contracts 911 can also include transfer logic for transferring corresponding assets, such as the cryptocurrency, fungible token, or NFT 902. This can involve defining one or more functions that take a new owner's address (e.g., wallet ID, network address, and/or the like) as an argument and updates the contract's internal state to reflect the transfer. Additionally or alternatively, the smart contracts 911 can include logic for calculating and distributing royalties, which may include one or more functions defined to obtain a sale price as an argument and uses it to calculate the royalty payment. In various implementations, individual smart contracts 911 specify functions, rules, policies, and/or configurations for verifying a user's 101 identity and/or for providing such verification to one or more orgs 133, 433. Additionally or alternatively, the smart contract 911 specifies transaction(s) or transaction type(s) that can be used to mint (generate), purchase, transfer, or otherwise obtain the corresponding NFT 911. The specific syntax and/or code of a smart contract may depend on the specific language being used and/or the platform or service 402, 800 on which the smart contract 911 is to be deployed. Smart contracts 911 can be written in higher-level programming languages and compiled to smart contract-specific bytecode. The higher-level programming languages may be a smart contract specific language such as, for example, Ethereum® Virtual Machine (EVM) bytecode, [Solidity], [Vyper] (Python derived), [Yul+], [Cadence], Bamboo, Lisp Like Language (LLL), EOS.IO, C++ for EOS.IO, Simplicity provided by Blockstream™, Rholang, Michelson, Counterfactual, Plasma, Plutus, Sophia, Serpent, Mutan, low-level Lisp-like language (LLL), and/or the like, or may be any of the other programming languages discussed herein, or combination(s) thereof. Once a smart contract 911 is written/developed and compiled, it can then be deployed to one or more blockchains 440, 915 via a suitable interface(s). Once deployed, one or more blockchain nodes in a blockchain network can execute or run the smart contract code.

In various implementations, a transaction is made on an NFT 902, the code of the smart contract 911 checks for certain conditions and executes relevant actions. This transaction is then stored as transaction content 917. For example, when an NFT 902 is requested by an entity/org 133, 433 that wishes to verify an individual's identity, the smart contract 911 will update and/or manage relevant permissions, and execute a transaction when permitted by the owner of the NFT 902 (e.g., a user 101 and/or user that operates user system/device 501, 810, 860). Some smart contracts 911 determine whether nodes, accounts, and/or platforms can access, or perform specific actions on or using a particular NFT 902, or on a particular blockchain 915 (see e.g., discussion of permissioning smart contracts in [EEA-CS7]). In some implementations, when an NFT 902 is used to verify a user's 101 identity, the corresponding smart contract 911 can automatically allocate payments or asset transfers to or from the owner of the NFT 902 based on the rates set and/or other conditions in the smart contract 911 when the NFT 902 was created or updated. Additionally or alternatively, when an NFT 902 is used to verify a user's 101 identity, the corresponding smart contract 911 can automatically transfer suitable data or indicators of the user's 101 identity to a requesting org 133, 433.

In some implementations, a smart contract 911 comprises a collection of code (e.g., its functions) and data (e.g., its state) that resides at a specific address on a blockchain(s) 440, 915. In Ethereum implementations, a developer publishes smart contract code into Ethereum Virtual Machine (EVM) memory. EVM is a global virtual computer whose state every participant on the Ethereum network stores and agrees on. Ethereum participants can request the execution of arbitrary code on the EVM, and code execution changes the state of the EVM. Individuals (e.g., wallet devices 810 and/or 860) can request smart contract code be executed by making a transaction request. An example smart contract is shown by table 1-1.

TABLE 1-1
example pseudocode for a smart contract

```
//SPDX-License-Identifier: MIT
pragma solidity \hat{ }0.7.0;
import “hardhat/console.sol”;
import “@openzeppelin/contracts/token/ERC721/ERC721.sol”;
import “@openzeppelin/contracts/utils/Counters.sol”;
contract NFTMinter is ERC721 {
  using Counters for Counters.Counter;
  Counters.Counter private _tokenIds;
  constructor(string memory tokenName, string memory symbol) ERC721(tokenName, symbol)
{
  _setBaseURI(“ipfs://”);
  }
  function mintToken(address owner, string memory metadataURI)
  public
  returns (uint256)
  {
  _tokenIds.increment( );
  uint256 id = _tokenIds.current( );
  _safeMint(owner, id);
  _setTokenURI(id, metadataURI);
  return id;
  }
}
```

In table 1-1, the pragma directive is used to enable certain compiler features or checks (see e.g., [Solidity], [OZContracts]). The contract is a function that is similar to classes in object oriented languages, which contains persistent data in state variables and functions that can modify these variables. Here, the contract is NFTMinter, which is set to be an ERC721 object, which is an implementation of [EIP-721] (note that [EIP-721] is also referred to as Ethereum Request for Comments 721 or “ERC721”). The ERC721 object comprises a set of interfaces, contracts, and utilities are all related to [EIP-721]. The constructor is a special function that is executed during the creation of the contract and cannot be called afterwards. In this case, the constructor sets the resource address or base URI (e.g., “ipfs://”) based on the token name (“tokenName”) and token symbol (“symbol”). In some implementations, the token name and token symbol are concatenated to generate the base URI. In some implementations, after the constructor has executed, the final code of the contract is stored on the blockchain.

Furthermore, the token name in table 1-1 refers to the name of the token being generated. In some implementations, a generic token name may be used for the NFTs 902 (e.g., “NFT-ID”). Additionally or alternatively, the token name may include a name of the particular sector for which the NFT is created (e.g., government sector 802, NGO sector 804, social media sector 806, or the like), a particular platform type for which the NFT 902 is created (e.g., social media platform, ecommerce platform, Web2, Web3, or other platform types), and/or a specific org for which the NFT 902 is created (e.g., a specific platform, enterprise, company, government agency or regulatory body, and/or the like).

The token symbol usually refers to the currency symbol (or currency sign) used to represent a currency (e.g., the symbol “$”representing U.S. dollars, the symbol “¥” representing Yen, the symbol “ [symbol] ” representing Bitcoin, the symbol “Ξ” representing Ether, and the like). In some implementations, such as when an NFT 902 is a government NFT 902, the symbols used for NFTs 902 may be based on the jurisdiction for which the NFT 902 is created (e.g., a two or three character country codes or geocodes as defined by ISO 3166-1:2020 and/or ISO 3166-2:2020, currency codes such as those defined by ISO 4217:2015, ITU letter codes, international telephone country codes, mobile country codes (MCCs), and/or the like). Additionally or alternatively, when an NFT 902 is an NGO/commercial NFTs, the symbols used for NFTs 902 may be based on an abbreviation or acronym of the org/entity, a ticker or stock symbol of the org/entity, and/or some other symbol as defined by the entity or org. Additionally or alternatively, a generic symbol may be used or the symbol may be omitted.

In some implementations, the smart contracts 911 may be part of one or more decentralized apps (Ðapps), which are apps running on a decentralized p2p network, often a blockchain 915. A ÐApp may include a user interface running on another (centralized or decentralized) system and may include decentralized storage and/or a message protocol and platform. In these implementations, a ÐApp may be a web user interface and a smart contract 911.

Furthermore, the SCE 910 and/or minting engine 920 may implement a suitable NFT framework 903, such as the Ethereum® platform (e.g., as discussed in [EIP-20], [EIP-165], [EIP-173], [EIP-721], [EIP-777], [EIP-1155], [EIP-3525], [EIP-5528], [EIP-5679], [EIP-5727], [EthereumDoc], [EEA-CS7], and the like), Ethereum® Immutable X platform, Polygon (formerly known as Matic Network), the Flow blockchain (see e.g., [Flow] and/or [Flow-NFT]), Bitcoin Cash, Cardano, and/or the like.

The metadata 913 may include any data about individual NFTs 902 such as, for example, NFT type and/or data identifying an asset/object to which the NFT represents (e.g., a particular type of ID that the NFT 902 represents), approval identities/entities, owners of an individual NFTs 902, an org associated with the NFT 902 (e.g., an issuer of the NFT 902), transferor ID (e.g., someone to which the NFT 902 is transferred, a description of the asset/object to which the NFT 902 represents (e.g., usage and/or permissions associated with the NFT 902), a URI pointing to a resource with an image (e.g., a MIME type image) representing the asset to which the NFT 902 represents, and/or other like metadata. In some implementations, the ID metadata 913 can reside on one or more blockchains 915, which may be stored in the NFT DB(s) 840. In other implementations, the ID metadata 913 is stored off-chain when a token URI includes the resource/location of the ID metadata 913, which may be stored either on a centralized server or in or at an IPFS location (e.g., a content identifier (CID) or other a label used to point to material in IPFS).

The IPFS microservice 914 may include APIs and/or functions to access data stored in the IPFS as discussed in [IPFS]. Additionally or alternatively, the IPFS microservice 914 includes an update feature, where an ID history can be updated without touching the original data. In these implementations, the update procedure may include reading or otherwise accessing current metadata from the IPFS; updating the metadata with new data entry/entries; adding new metadata to the IPFS; and updating content hash on smart contract.

The minted NFTs 902 may be stored in one or more blockchains 915. Some or all of the blockchains 915 may be private blockchains 915 and/or some or all of the blockchains 915 may be public blockchains 915. In some implementations, individual blockchains 915 are owned or are otherwise associated with a particular org or sector. For example, a blockchain 915 owned or otherwise corresponding to a State's Department of Motor Vehicles (DMV) may include blocks for respective driver's license NFTs 902. In other implementations, individual blockchains 915 are owned or are otherwise associated with a an individual, where each block corresponds to minted NFT 902 for that person, and new blocks are added whenever the NFT 902 is updated with new or alternative user data 410.

The transaction content 917 includes content or data associated with transactions involving the minted NFTs 902. In general, a transaction 917 involves a request to execute operations on a blockchain 915 that changes the state of one or more accounts. More specifically, transaction content 917 may include data committed to a blockchain 915, which may be signed by an originating account and targeting a specific address. The transaction content 917 also contains metadata 913. Some transaction content 917 may include a contract creation transaction, which is a special transaction 917 with a zero address (e.g., an address of all zeros, “0”) as the recipient, which is used to register a contract and record it on the Ethereum blockchain.

Nodes processing transactions is/are the basis of adding blocks to a blockchain 915, and may be subject to the permissions defined in the corresponding smart contracts 911. When mining new blocks, a processing node checks the validity of a transaction using the appropriate smart contract 911 with the state at the block's parent. If not permitted, the transaction is discarded. If permitted, the transaction is included as a new block and the block dispatched to other nodes (for inclusion in local versions of the blockchain 915). When receiving a block, the receiving node checks each included transaction using the appropriate smart contract 911 with the state at the block's parent. If any transactions in the new block are not permitted, the block is considered invalid and discarded. If all transactions are permitted, the block passes the permissioning validation check and is then subject to any other validity assessments the client 810, 860 might usually perform.

Transactions 917 can be on-chain transactions 918 or off-chain transactions 919. On-chain transactions 918 (simply referred to as “transactions 918” or “on-chain 918”) are transactions that occur on a blockchain 915 that are reflected on the distributed ledger. On-chain transactions 918 are transactions that have been validated or authenticated and lead to an update to the overall blockchain network. An on-chain transaction 918 occurs and is considered valid when the blockchain 915 is modified. By contrast, an off-chain transaction 919 (simply referred to as “off-chain 919”) takes the value outside of the blockchain 915 and/or transactions that do not occur on the blockchain network, but instead, are transacted on another electronic system.

The minting engine 920 is a microservice or facility that generates NFTs 902. Similar to the way metal coins are minted and put into circulation, the NFTs 902 are also “minted” after they are created. The minting process turns the collection of inputs 901 into an NFT 902. During the minting process, the owner of the NFT 902 can schedule reviews, define and/or execute identity verification processes, and/or the like. Once the NFT 902 is generated, the issuance microservice 916 issues or otherwise distributes the NFT 902 to the wallet 810 of the owner of the NFT 902. Minting NFTs typically involves the use of a smart contract 911 that is specifically designed for minting NFTs 902. Here, the smart contract code defines rules and/or functions for the NFT 902, and how the NFT 902 will be minted and transferred. Additionally, the smart contract 911 may define the structure of the NFT 902, such as the particular data/metadata fields the NFT 902 will include (e.g., tokenId, owner, name, description, traits, HP account numbers, the owner's financial information, external links to relevant content, any of the metadata 913, and/or the like). In some implementations, once the smart contract 911 is written or developed, the smart contract 911 is deploy to a blockchain 915. This is done using an SDK, command line interface (CLI), NFT minting app, and/or other suitable interface provided by the blockchain/NFT platform (e.g., NFT service(s) 402, 800 and/or the like). Once the smart contract 911 is deployed, it can be used to mint the NFT 902.

The NFT minting can involve calling specific function(s) in the smart contract (e.g., mint ( ) and/or the like), and passing data and/or parameters (e.g., a set of inputs 901) to the smart contract 911 for minting the NFT 902. Calling the specific function(s) generates a unique ID for the new token/NFT (e.g., “tokenId”), and assigns the new token/NFT (or the unique ID) to an address of the NFT's 902 owner by, for example, updating the ownership information within the smart contract 911 to include the owner's address. Additionally, the function(s) may update the smart contract 911 to include the relevant metadata (e.g., metadata 913) in the predefined fields, and/or reference an off-chain storage location for the metadata (e.g., metadata 913). In some implementations, the smart contract 911 emits an event (e.g., a transfer event or the like) to notify the blockchain network (or individual blockchain nodes) about the minting transaction. This event can contain information (e.g., transaction content 917), such as the sender, receiver, the token ID (or unique ID), and/or other data. The minting transaction indicated by the event message is broadcast to blockchain nodes (or “miners”) that validate and/or verify the transaction data (e.g., transaction content 917) and include the generated NFT 902 as a block in the blockchain. When the transaction is confirmed and included in a block, the NFT 902 is officially minted, and ownership is transferred to the specified address. The specific methods, operations, tasks, and/or functions for minting an NFT 902 may depend on the NFT/blockchain platform, the specific smart contract 911 being used, and/or other conditions, parameters, and/or criteria. In some cases, minting an NFT 902 requires payment of transaction fees on the same or different blockchain 915, which may be in the form of fiat currency and/or cryptocurrency. In some implementations, the fees can vary depending on the NFT/blockchain platform, network conditions, and/or other conditions, parameters, and/or criteria.

In some implementations, each NFT 902 is identified by a unique ID inside a smart contract 911. This unique ID does not change for the life of the smart contract 911. An attribute-value pair including the contract address and the tokenId is a globally unique and fully-qualified identifier for a specific asset on a blockchain 915. The unique ID may be any value or data structure that uniquely identifies an individual and/or their user devices 810, 860. In one example, the unique ID may be a randomly generated number or string, which may be generated using a suitable random number generator, pseudorandom number generators (PRNGS), and/or the like. Additionally or alternatively, the unique ID may be a version 4 Universally Unique Identifier (UUID) that is randomly generated according to Leach et al., “A Universally Unique IDentifier (UUID) URN Namespace”, Internet Engineering Task Force (IETF), Network Working Group, Request for Comments (RFC): 4122 (July 2005) (“[RFC4122]”), which is hereby incorporated by reference in its entirety and for all purposes. In one example implementation, the random unique ID is generated for an individual device 810, 860 upon completing the registration process.

Additionally or alternatively, the unique ID may be a hash value calculated from one or more inputs (which may or may not be unique to the individual/user devices 810, 860). In one example implementation, the unique ID may be generated using the supplied HCD 110 (or a portion thereof) and/or a National Provider Identifier (NPI) as an input to a suitable hash function (e.g., such as sha3 and/or any of those discussed herein). For example, the unique ID may be a version 3 or 5 UUID that is generated according by hashing a namespace identifier and name using MD5 (UUID version 3) or SHA-1 (UUID version 5) as discussed in [RFC4122]. In another example, the unique ID may be generated using any suitable hash function and using any combination of PII and/or device or system information supplied by a user and/or extracted from the user devices 810, 860 during the NFT generation process.

Additionally or alternatively, the unique ID may be a digital certificate supplied by a suitable certificate authority, or may be generated using the digital certificate (e.g., hashing the digital certificate). In another embodiment, the unique ID may be a specific identifier or may be generated using the specific identifier (e.g., as discussed previously). The specific identifier may be any suitable identifier associated with a user and/or user devices 810, 860, associated with a network session, an application, an app session, an app instance, an app-generated identifier, and/or some other identifier (ID). The specific identifier may be a user ID or unique ID for a specific user on a specific client app and/or a specific user devices 810, 860. Additionally or alternatively, the user ID may be or include one or more of a user ID (UID) (e.g., positive integer assigned to a user by a Unix-like OS), effective user ID (euid), file system user ID (fsuid), saved user id (suid), real user id (ruid), a cookie ID, a realm name, domain ID, logon user name, network credentials, social media account name, user session ID, and/or any other like ID associated with a particular user devices 810, 860. Additionally or alternatively, the specific identifier may be a device identifier such as a device ID, product ID/code, serial number of the user devices 810, 860, a document of conformity (DOC), and/or the like. Additionally or alternatively, the specific identifier may be a network ID such as an international mobile subscriber identity (IMSI), internet protocol (IP) address, and/or some other suitable network address such as those discussed herein. Any of the aforementioned identifiers and/or information may be combined to produce the UID/unique ID, and/or other information, such as the information discussed infra, may be used to produce the UID/unique ID.

Additionally or alternatively, the unique ID may be based on a device fingerprint of the user devices 810, 860. The device fingerprint may be any information collected about the software and hardware of a computing device for the purpose of identification, which may or may not be incorporated into an identifier (e.g., any of the aforementioned IDs or the like). In one example implementation, the device fingerprint may be based on system data, sensor data, and/or the like that is/are collected and combined using some known mechanism (e.g., a hash function or the like). In another example implementation, the device fingerprint may be the output of a physical unclonable function (PUF) implemented by a tamper-resistant chipset in the user devices 810, 860 (e.g., TEE 1109 of FIG. 11 discussed infra). When a physical stimulus (e.g., electric impulse) is applied to the PUF, it may react in an unpredictable way due to the complex interaction of the stimulus with the physical microstructure of the PUF. This exact microstructure may depend on physical factors introduced during manufacture which may be unpredictable. The PUF outputs the device fingerprint that may serve as the UID. Any of the aforementioned embodiments/implementations may be combined and/or used to generate the NFT 902.

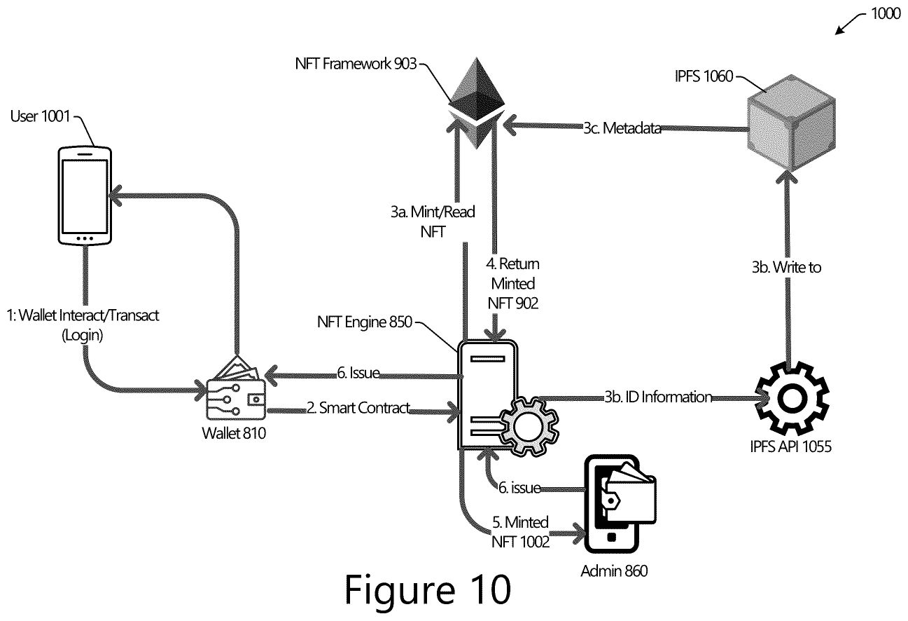

FIG. 10 depicts an example process 1000 for minting NFTs 902. Process 1000 begins at operation 1 where a user 1001 logs in to their wallet app (e.g., wallet 810). As mentioned previously, the wallet 810 could be a mobile app (e.g., iOS/Android app), a browser extension or plugin, a web app, a desktop app, and/or the like.

At operation 2, the smart contract 911 is triggered. The smart contract 911 is inside and a part of the NFT engine 850, where the admin 860 defines the requirements and functions of each NFT type, then creates it. Deployment scripts are also included here. In some implementations, the smart contracts 911 are written in Solidity or some other suitable smart contract language, such as any of those mentioned herein. The admin 860 may code, compile, deploy, and run this smart contract 911. The power, flexibility, and multiuse may come from proprietary coding used for generating such smart contracts 911.

At operation 3a, the NFT engine 850 (or the minting engine 920) mints a new NFT 902. To create a new NFT 902, the user 810 or the admin 860 gathers HCD 110, 410 provided by the user 1001 (e.g., EHR/KBA data, metadata 913, and/or the like), encrypts the information, and then uploads the information to IPFS 1060 via the IPFS API 1055 at operation 3b, and/or uploads the information to a private data storage server (e.g., DB server 830 and/or NFT/blockchain DB(s) 840). In some examples, the IPFS 1060 is stored in the NFT/blockchain DB(s) 840, or otherwise corresponds to the NFT/blockchain DB(s) 840. Additionally, the information stored in the IPFS 1060 or the private data storage server may correspond to the metadata 913 of FIG. 9. This information (e.g., metadata 913) is provided to the NET framework 903 at operation 3c. This will also return a URL or other reference that points to the storage location of the data (e.g., metadata 913) at operation 4. In some implementations, this data (e.g., HCD 110, user data 410, and/or metadata 913) is not stored on the blockchain 915.

Examples of user data 410 that may be submitted to the admin 860 and/or the NFT engine 850 include ID documents, phone number, email address; social media handles, YouTube channel, and/or the like; work or school ID; residential, academic, and/or financial history/records and/or related documentations (these can be periodically updated if needed); EHR data; KBA data; any other information the users 810, 860 and/or orgs 133, 433 require; and/or any other information or data discussed herein. The minting process stores the URL or other reference to the metadata 913 inside the NFT 902.

When an NFT 902 is minted, it is added to the Admin digital wallet 860 at operation 5. A newly minted NFT 902 belongs initially to whoever did the actual minting, which in this example is the admin 860. In some examples, the admin 860 may represent an employee, agent, or department of an org 133, 433 that requests a EHR NFT on behalf of user 1001. The NFT 902 is then transferred to the individual's wallet 810 during the issuance process described infra.

At operation 6, the minted NFT 902 is issued to the wallet 810. The admin 860 issues the new NFT 902 by transferring the newly minted NFT 902 to the wallet 810 of the new ID holder 1001 via the NFT engine 850 or using some other transfer mechanism. The transfer of the minted NFT 902 from the admin wallet 860 to the wallet 810 of the new NFT holder 1001 may be recorded as transaction content 917. In various implementations, an individual 1001 can have multiple NFTs 902, for example, a first NFT 902 for a first HP account, a second NFT 902 for a second HP account, a third NFT 902 for a third HP account, and so forth.

### 2. Example Hardware and Software Systems and Configurations

Referring back to FIGS. 1-10, the client devices 501, 810, 860, 1001 (hereinafter, the “client devices”, “client systems,” “user systems,” “user devices,” and/or the like) include physical hardware devices and software components capable of accessing content and/or services provided by the NFT/blockchain service 800. In order to access the content/services, the client devices include components such as processors, memory devices, communication interfaces, and the like. Additionally, the client devices may include, or be communicatively coupled with, one or more sensors (e.g., image capture device(s), microphones, and/or the like), which is/are used to capture biometric data. The client devices communicate with the NFT/blockchain service 800 to obtain content/services using any suitable access technology and/or communication protocols, such as any of those discussed herein. In this regard, a client device may establish a session with the NFT/blockchain service 402, 800 to exchange information/data with the NFT/blockchain service 402, 800. The session may be bound by use of a session secret (e.g., a password, digital certificate, etc.) that the subscriber's software (e.g., a browser, app, OS, or the like).

The client devices can be implemented as any suitable computing system or other data processing apparatus usable by users to access content/services provided by the NFT/blockchain service 402, 800. In the examples of FIGS. 1-10, the client devices are depicted as digital wallets or mobile cellular phones (e.g., a “smartphones”); however, the client devices can be embodied as any other type computer device/system, such as laptops, tablets, desktop computers, workstations, portable media players, wearable devices (e.g., smart watches and/or the like), video game consoles, digital media players, handheld messaging devices, personal data assistants, electronic book readers (or “e-readers”), extended reality (XR) devices (including augmented reality (AR), virtual reality (VR), and/or mixed reality (MR) devices), in-vehicle infotainment (IVI) systems, in-car entertainment (ICE) systems, instrument clusters (IC), head-up display (HUD) devices, on-board diagnostic (OBD) devices, dashtop mobile equipment (DME), Electronic Engine Management System (EEMS), electronic control unit (ECU), electronic control modules (ECM), embedded systems, microcontrollers, control modules engine management systems (EMS), networked or “smart” appliances, machine-to-machine (M2M) and/or Internet of Things (IoT) devices, autonomous sensors, servers (e.g., stand-alone, rack-mounted, blade, and/or the like), cloud compute nodes, edge compute nodes, network elements, network appliances, base stations, access points, and/or any other electronic devices.

In some examples, the NFT/blockchain service 402, 800 may represent a cloud computing service, an intranet, enterprise network, or some other like private network that is unavailable to the public. In one example implementation, the entirety of NFT/blockchain service 402, 800 including both the front end and the back end may be implemented in or by a cloud computing service (e.g., a “full stack” cloud implementation). The cloud computing service (or “cloud”) includes networks of physical and/or virtual computer systems (e.g., one or more servers), data storage systems/devices, and/or the like within or associated with a data center or data warehouse that provide access to a pool of computing resources. The one or more servers in a cloud include user computer systems, where each of the servers includes one or more processors, one or more memory devices, input/output (I/O) interfaces, communications interfaces, and/or other like components. The servers may be connected with one another via a Local Area Network (LAN), fast LAN, message passing interface (MPI) implementations, and/or any other suitable networking technology. Various combinations of the servers may implement different cloud elements or nodes, such as cloud manager(s), cluster manager(s), master node(s), one or more secondary (slave) nodes, and the like. The one or more servers may implement additional or alternative nodes/elements in other implementations. In some cloud implementations, at least some of the servers in the cloud (e.g., servers that act as secondary nodes) may implement app server and/or web server functionality, which includes, inter alia, obtaining various messages from the client devices; processing data contained in those messages; routing data to other nodes in the cloud for further processing, storage, retrieval, etc.; generating and communicating messages including data items, content items, program code, renderable webpages and/or electronic documents (e.g., including the various GUIs discussed herein), and/or other information to/from client devices; and/or other like app server functions. In this way, various combinations of the servers may implement different cloud elements/nodes configured to perform the implementations discussed herein.

The NFT/blockchain service 402, 800 includes one or more servers 820, 830, and/or 850 and one or more DBs (e.g., NFT DB(s) 840 or the like). The web server(s) (e.g., servers 820) serve static content from a file system of the web server(s). The servers 820, 830, and/or 850 may be virtual or physical systems that provide NFT/blockchain services to individual users (e.g., using a client system(s) 810, 860) and/or for org 133, 433 platforms. In some implementations, some or all of the NFT/blockchain services may be provided by or accessed from third party systems/services, and the information provided by the third party systems/services may be enhanced or amended using information collected by the NFT/blockchain service 402, 800. The virtual and/or physical systems may include app servers, web servers, and/or other like computing systems/devices. The particular NFT/blockchain services provided by the servers 820, 830, and/or 850 may depend on the architecture or implementation of the NFT/blockchain service 402, 800, and may vary from embodiment to embodiment. In one example, one or more of the servers 820, 830, and/or 850 may operate as an app server and may provide respective NFT/blockchain services (e.g., EHR onboarding and data collection, EHR NFT minting, EHR ID minting, transaction monitoring and notifications, and so forth) as separate processes, or by implementing autonomous software agents. In another example, individual NFT server(s) 820, 830, and/or 850 may be dedicated to perform separate NFT/blockchain services, where app servers 820 are used to obtain requests from client devices and provide information/data to the NFT server(s) 830 and/or 850 to perform their respective NFT related services.

The app servers 820 comprise one or more physical and/or virtualized systems for providing content and/or functionality (e.g., services) to one or more clients (e.g., client system) over a network. The physical and/or virtualized systems include one or more logically or physically connected servers and/or data storage devices distributed locally or across one or more geographic locations. Generally, the web/app servers 820 are configured to use IP/network resources to provide web pages, forms, apps, data, services, and/or media content to client system. Additionally or alternatively, the web/app servers 820 may generate and serve dynamic content (e.g., server-side programming, database connections, dynamic generation of web documents) using an appropriate plug-in and/or server-side apps. The app server(s) 820 may implement an app platform, which is a framework that provides for the development and execution of server-side apps as part of an app hosting service. The app platform enables the creation, management, and execution of one or more server-side apps developed by or for the NFT/blockchain service 402, 800 and/or third party app developers, which allow users and/or third party app developers to access the NFT/blockchain service 402, 800 via respective client systems. The client devices may operate respective client apps (e.g., app 1151 in FIG. 11) to access the dynamic content, for example, by sending appropriate HTTP messages or the like, and in response, the server-side app(s) may dynamically generate and provide source code documents to the client app, and the source code documents are used for generating and rendering graphical objects (or simply “objects”) within the client app. The server-side apps may be developed with any suitable server-side programming languages or technologies, such as PHP; Java™ based technologies such as Java Servlets, JavaServer Pages (JSP), JavaServer Faces (JSF), etc.; ASP.NET; Ruby or Ruby on Rails; and/or any other like technology that renders HyperText Markup Language (HTML), such as those discussed herein. The apps may be built using a platform-specific and/or proprietary development tool, and/or programming languages.

The servers 820, 830, and/or 850 serve one or more instructions or source code documents to client devices, which may then be executed within a client app (e.g., a wallet app as discussed previously and/or app 1151 in FIG. 11) to render one or more objects (e.g., GUIs). The GUIs can include graphical control elements (GCEs) that allow the client devices to perform various functions and/or to request or instruct the NFT/blockchain service 402, 800 to perform various functions. The servers 820, 830, and/or 850 may provide various interfaces such as those discussed herein. The interfaces and/or apps/GUIs/GCEs may be developed using website development tools and/or programming languages (e.g., HTML, Cascading Stylesheets (CSS), JavaScript, Jscript, Ruby, Python, etc.) and/or using platform-specific development tools (e.g., Android® Studio™ integrated development environment (IDE), Microsoft® Visual Studio® IDE, Apple® iOS® software development kit (SDK), Nvidia® Compute Unified Device Architecture (CUDA)® Toolkit, Flow® playground IDE, Remix IDE, ChainIDE, Azure Blockchain Workbench provided by Microsoft®, IntelliJ IDEA® provided by JetBrains, and/or any other suitable SDK/IDE, such as any of those discussed herein). The term “platform-specific” may refer to the platform implemented by the client systems and/or the platform implemented by the servers 820, 830, and/or 850. Example interfaces are shown and described with w.r.t FIGS. 1-11. In an example implementation, the servers 820, 830, and/or 850 may implement Apache HTTP Server (“httpd”) web servers or NGINX™ webservers on top of the Linux® OS. In this example implementation, PHP and/or Python may be employed as server-side languages, MySQL may be used as the DQL/DBMS. In an example implementation, the mobile apps may be developed for Android®, iOS®, and/or some other mobile platform.

In some embodiments, the one or more servers 820, 830, 850 may implement or operate one or more intelligent agent(s) (also referred to as “artificial intelligence (AI) agent(s),” “AI/ML inference function(s),” “inference engine(s),” “AI/ML entities”, and/or the like) to perform the EHR services/processes 235 and/or the NFT/blockchain services 402 discussed previously, or portions thereof. The intelligent agent(s) may operate one or more AI/ML models to generate inferences based on data collected from client systems, servers 820, 830, 850, NFT/blockchain service 402, 800, and/or other sources. The AI/ML models can include any suitable architecture, network, topology, configuration, and/or arrangement, including any of those mentioned herein and/or mentioned in '074 and/or '081. The intelligent agent(s) is/are implemented as autonomous software agents, implemented using hardware elements, or a combination thereof, which are executed or otherwise operated by one or more processors (e.g., processor(s) 1101, accelerator(s) 1110, and/or the like) of one or more servers 820, 830, 850.

Furthermore, one or more servers 820, 830, 850 may digitally sign, encrypt/decrypt data, and/or hash data using a cryptographic mechanism. Examples of cryptographic mechanisms include cryptographic hash algorithms (e.g., a function in the Secure Hash Algorithm (SHA) 2 set of cryptographic hash algorithms (e.g., SHA-226, SHA-256, SHA-512, and the like), SHA 3, and so forth, or any type of keyed or unkeyed cryptographic hash function, and/or any other function discussed herein); an elliptic curve cryptographic (ECC) algorithm (e.g., Elliptic Curve cryptography Key Agreement algorithm (ECKA) algorithm, Elliptic Curve cryptography Digital Signature Algorithm (ECDSA), Lenstra elliptic-curve factorization or elliptic-curve factorization method (ECM), Menezes-Qu-Vanstone (MQV) or elliptic curve MQV (ECMQV), Elliptic Curve Diffie-Hellman (ECDH) key agreement, Elliptic Curve Integrated Encryption Scheme (ECIES) or Elliptic Curve Augmented Encryption Scheme, Edwards-curve Digital Signature Algorithm (EdDSA), and/or the like); Rivest-Shamir-Adleman (RSA) cryptography; Merkle signature scheme, advanced encryption system (AES) algorithm; a triple data encryption algorithm (3DES); Quantum cryptography algorithms; and/or the like.

The NFT DB 840 may be stored in one or more data storage devices or storage systems that act as a repository for persistently storing and managing collections of data according to one or more predefined DB structures. The data storage devices/systems may include one or more primary storage devices, secondary storage devices, tertiary storage devices, non-linear storage devices, and/or other like data storage devices. In some implementations, at least some of the servers 820, 830, and/or 850 may implement a suitable database management system (DBMS) to execute storage and retrieval of information against various database object(s) in the NFT DB 840. These servers 820, 830, and/or 850 may be storage servers, file servers, or other like computing systems. The DBMS may include a relational database management system (RDBMS), an object database management system (ODBMS), a non-relational DBMS (e.g., a NoSQL DB system), and/or some other DBMS used to create and maintain the NFT DB 840. The NFT DB 840 can be implemented as part of a single database, a distributed database, a collection of distributed databases, a database with redundant online or offline backups or other redundancies, etc., and can include a distributed database or storage network. These servers 820, 830, and/or 850 may implement one or more query engines that utilize one or more data query languages (DQLs) to store and retrieve information in/from the NFT DB 840, such as Structured Query Language (SQL), Structured Object Query Language (SOQL), Procedural Language/SOQL (PL/SOQL), GraphQL, Hyper Text SQL (HTSQL), Query By Example (QBE), object query language (OQL), object constraint language (OCL), non-first normal form query language (NIQL), XQuery, and/or any other DQL or combinations thereof. The query engine(s) may include any suitable query engine technology or combinations thereof including, for example, direct (e.g., SQL) execution engines (e.g., Presto SQL query engine, MySQL engine, SOQL execution engine, Apache® Phoenix® engine, etc.), a key-value datastore or NoSQL DB engines (e.g., DynamoDB® provided by Amazon.com®, MongoDB query framework provided by MongoDB Inc.®, Apache® Cassandra, Redis™ provided by Redis Labs®, etc.), MapReduce query engines (e.g., Apache® Hive™, Apache® Impala™ Apache® HAWQ™, IBM® Db2 Big SQL®, etc. for Apache® Hadoop® DB systems, etc.), relational DB (or “NewSQL”) engines (e.g., InnoDB™ or MySQL cluster™ developed by Oracle®, MyRocks™ developed by Facebook.com®, FaunaDB provided by Fauna Inc.), PostgreSQL DB engines (e.g., MicroKernel DB Engine and Relational DB Engine provided by Pervasive Software®), graph processing engines (e.g., GraphX of an Apache® Spark® engine, an Apache® Tez engine, Neo4J provided by Neo4j, Inc.™, etc.), pull (iteration pattern) query engines, push (visitor pattern) query engines, transactional DB engines, extensible query execution engines, package query language (PaQL) execution engines, LegoBase query execution engines, and/or some other query engine used to query some other type of DB system (such as any processing engine or execution technology discussed herein). In some implementations, the query engine(s) may include or implement an in-memory caching system and/or an in-memory caching engine (e.g., memcached, Redis, and/or the like) to store frequently accessed data items in a main memory of the servers 820, 830, 850 for later retrieval without additional access to the persistent data store. Suitable implementations for the database systems and storage devices are known or commercially available, and are readily implemented by persons having ordinary skill in the art.

The NFT DB 840 stores or otherwise includes a plurality of database objects (DBOs). The DBOs may be arranged in a set of logical tables containing data fitted into predefined or customizable categories, and/or the DBOs may be arranged in a set of blockchains or ledgers wherein each block (or DBO) in the blockchain is linked to a previous block. Each of the DBOs may include data associated with users/client devices, such as user/client IDs, UIDs, NFT IDs, EHR NFTs, and/or, in certain embodiments, other data such as biographic data; biometric data; data collected from various external sources; identity session identifiers (IDs); and/or other like data. Additionally or alternatively, the NFT DB 840 may store. Some of the DBOs may store information pertaining to relationships between any of the data items discussed herein. Some of the DBOs may store permission or access-related information for each user. These DBOs may indicate specific third parties that are permitted to access identity data of a particular user. In some implementations, the permission or access-related DBOs for each user may be arranged or stored as a blockchain to control which third parties can access that user's identity data. In these embodiments, the blockchain(s) do not actually store user biometric and/or biographic data, but instead are used to authorize specific third party platforms to access specific identity data items and to track or account for the accesses to the identity data items.

As alluded to previously, the client device(s) is/are configured to run, execute, or otherwise operate the client app (e.g., app 1151 in FIG. 11). The client app is a software app designed to generate and render objects, which include various types of content. At least some of the objects include GUIs and/or GCEs that enable interactions with the NFT/blockchain service 402, 800. In some embodiments, the client app is an app container/skeleton in which an NET app operates (e.g., a wallet app, payment app, web browser, and the like). For example, the objects may represent a web app that runs inside the client app, and the client app may be an HTTP client, such as a “web browser” (or simply a “browser”) for sending and receiving HTTP messages to and from a web/app servers 820 of the NFT/blockchain service 402, 800. In some examples, a suitable browser extension or plug-in may be configured to allow the client app to render objects that allow the user to interact with the NFT/blockchain service 402, 800 for generating EHR NFTs 902 according to the embodiments discussed herein. Such browsers can include a suitable browser engine such as, for example, a trident engine (e.g., Microsoft® Internet Explorer®, Tencent® Traveler®, and/or the like), a gecko engine (e.g., Mozilla® Firefox®, Tor Browser, and/or the like), a WebKit engine (e.g., Apple® Safari®, the Nintendo® NetFront Browser NX®, Google® Chrome® for iOS, and/or the like), blink engine (e.g., Google® Chrome®, Opera®, Microsoft® Edge®, and/or the like), Presto-based (e.g., Nintendo® DS® browser, and/or the like), and/or proprietary browser engines (e.g., Ekioh® Flow®, the play station 5 (PS5) secret web browser, and/or the like). In some examples, one or more client app(s) are native app(s) (e.g., executed and rendered in a container), web apps) (e.g., executed and rendered inside a browser), or hybrid apps (e.g., web apps executed/rendered in a native/platform container or skeleton), or variants thereof. In these examples, an owner/operator of NFT/blockchain service 402, 800 may have pre-built a client app(s) (e.g., wallet device and/or app) for use by users of compute devices 501 to access their specific apps and/or services. In some embodiments, the client app is an app specifically developed or tailored to interact with the NFT/blockchain service 402, 800 NFT/blockchain service 402, 800. For example, the client app may be a desktop or native (mobile) app that runs directly on the client system(s) without a browser, and which communicates (sends and receives) suitable messages with the NFT/blockchain service 402, 800. In some embodiments, the client app is an app specifically developed or tailored to interact with the NFT/blockchain service 402, 800 for generating NFTs 902 (e.g., NFT IDs, EHR NFTs, and/or the like).

The client app (e.g., app 1151 in FIG. 11) may be developed using any suitable programming languages and/or development tools, such as those discussed herein or others known in the art. The client app may be platform-specific, such as when the client system(s) is/are implemented as a mobile device, such as a smartphone, tablet computer, or the like. In these embodiments, the client app may be a mobile web browser, a native app (or “mobile app”) specifically tailored to operate on the mobile client system(s), or a hybrid app wherein objects (or a web app) is embedded inside the native app. In some implementations, the client app and/or the web apps that run inside the client app is/are specifically designed to interact with server-side apps implemented by the app platform of the provider system (discussed infra). In some implementations, the client app, and/or the web apps that run inside the client app may be platform-specific or developed to operate on a particular type of client system(s) or a particular (hardware and/or software) client system(s) configuration. The term “platform-specific” may refer to the platform implemented by the client system(s), the platform implemented by the NFT/blockchain service 402, 800, and/or a platform of a third party system/platform.

In the aforementioned embodiments, the client system(s) implementing a client app is capable of controlling its communications/network interface(s) to send and receive HTTP messages to/from the NFT/blockchain service 402, 800, render the objects in the client app, request connections with other devices, and/or perform (or request performance) of other like functions. The header of these HTTP messages includes various operating parameters and the body of the HTTP messages include program code or source code documents (e.g., HTML, XML, JSON, and/or some other like object(s)/document(s)) to be executed and rendered in the client app. The client app executes the program code or source code documents and renders the objects (or web apps) inside the client app.

The rendered objects (or executed web app) allow the user of the client system(s) to view content provided by the NFT/blockchain service 402, 800, which may include the results of a requested service, visual representations of data, hyperlinks or links to other resources, EHR NFTs (see e.g., 605a in FIG. 6), and/or the like. The rendered objects also include interfaces for interacting with the NFT/blockchain service 402, 800, for example, to request additional content or services from the NFT/blockchain service 402, 800. In an example, the rendered objects may include GUIs, which are used to manage the interactions between the user of the client system(s) and the NFT/blockchain service 402, 800. The GUIs comprise one or more GCEs (or widgets) such as buttons, sliders, text boxes, tabs, dashboards, etc. The user of the client system(s) may select or otherwise interact with one or more of the GCEs (e.g., by pointing and clicking using a mouse, or performing a gesture for touchscreen-based systems) to request content or services from the NFT/blockchain service 402, 800.

In some cases, the user of client system(s) may be required to authenticate their identity in order to obtain content and/or services from the NFT/blockchain service 402, 800. To provide the NFT/blockchain services to the user, the client app may be, or may include, a secure portal to the NFT/blockchain service 402, 800 (e.g., a front-end portal and/or the like). The secure portal may be a stand-alone app, embedded within a web or mobile app (wallet app) provided by NFT/blockchain service 402, 800, and/or invoked or called by the web/mobile app provided by NFT/blockchain service 402, 800 (e.g., using an API, remote procedure call (RPC), inter-process communications (IPC), and/or the like). In these cases, graphical objects rendered and displayed within the client app may be a GUI and/or GCEs of the secure portal, which allows the user to share data (e.g., HCD 110 and/or user data 410) with the NFT/blockchain service 402, 800. The secure portal also allows users to access and/or perform various NFT-related tasks. For example, the secure portal may provide access to a dashboard GUI that allows admins 860 to submit HCD 110, user data 410, queries for individuals or entities, set AML policies/rules, and/or setup or subscribe to notifications for automatically receiving alerts for suspicious activities for individuals or entities.

Additionally or alternatively, the client app may collect various data from the client device(s) without direct user interaction with the client app. For example, the client app may cause the client system(s) to generate and transmit one or more HTTP messages with a header portion including, inter alia, an IP address of the client system(s) in an X-Forwarded-For (XFF) field, a time and date that the message was sent in a Date field, and/or a user agent string contained in a User Agent field. The user agent string may indicate an operating system (OS) type/version being operated by the client system(s), system information of the client system(s), an app version/type or browser version/type of the client app, a rendering engine version/type implemented by the client app, a device and/or platform type of the client system(s), and/or other like information. These HTTP messages may be sent in response to user interactions with the client app (e.g., when a user submits biographic or biometric data as discussed infra), or the client app may include one or more scripts, which when executed by the client system(s), cause the client system(s) to generate and send the HTTP messages upon loading or rendering the client app. Other message types may be used and/or the user and/or client system(s) information may be obtained by other means in other embodiments.

In addition to (or alternative to) obtaining information from HTTP messages as discussed previously, the servers 820, 830, 850 may determine or derive other types of user information associated with the client system(s). For example, the servers 820, 830, 850 may derive a time zone and/or geolocation in which the client system(s) is/are located from an obtained IP address. In some embodiments, the user and/or client system(s) information may be sent to the servers 820, 830, 850 when the client system(s) loads or renders the client app. For example, the login page may include JavaScript or other like code that obtains and sends back information (e.g., in an additional HTTP message) that is not typically included in an HTTP header, such as time zone information, global navigation satellite system (GNSS) and/or Global Positioning System (GPS) coordinates, screen or display resolution of the client system(s), and/or other like information. Other methods may be used to obtain or derive such information in other embodiments.

FIG. 11 illustrates an example compute node 1100 (also referred to as “platform 1100,” “device 1100,” “appliance 1100,” “system 1100”, and/or the like), and various components therein, for implementing the techniques (e.g., operations, processes, methods, and methodologies) described herein. In FIG. 11, like numbered items are the same as discussed previously w.r.t FIGS. 1-10. The compute node 1100 provides a closer view of the respective components of node 1100 when implemented as or as part of a computing device or system. The compute node 1100 can include any combination of the hardware and/or logical components referenced herein, and may include or couple with any device usable with a communication network or a combination of such networks. Any combination of components depicted by FIG. 11 can be implemented as individual ICs, discrete electronic devices, or other modules, instruction sets, programmable logic or algorithms, hardware, software, firmware, or a combination thereof adapted in the compute node 1100, or as components otherwise incorporated within a chassis of a larger system. Additionally or alternatively, any combination of the components depicted by FIG. 11 can be implemented as a system-on-chip (SoC), a single-board computer (SBC), a system-in-package (SiP), a multi-chip package (MCP), and/or the like, in which a combination of the hardware elements are formed into a single IC or a single package.

The compute node 1100 includes physical hardware devices and software components capable of providing and/or accessing content and/or services to/from the remote system 1190. The compute node 1100 and/or the remote system 1190 can be implemented as any suitable computing system or other data processing apparatus usable to access and/or provide content/services from/to one another. The compute node 1100 communicates with remote systems 1190, and vice versa, to obtain/serve content/services using any suitable communication protocol, such as any of those discussed herein. In some implementations, the remote system 1190 may have some or all of the same or similar components as the compute node 1100. As examples, the compute node 1100 and/or the remote system 1190 can be embodied as desktop computers, workstations, laptops, mobile phones (e.g., “smartphones”), tablet computers, portable media players, wearable devices, head-up display device (HUD) devices, extended reality (XR) devices (e.g., including virtual reality (VR), augmented reality (AR), and/or mixed reality or mediated reality (MR) technologies), in-vehicle compute systems, smart appliances, smart factory machinery, network infrastructure elements, network appliances, network access nodes (NANs), robots, drones, sensor systems and/or IoT devices, server(s), cloud compute nodes, edge compute nodes, an aggregation of computing resources (e.g., in a cloud-based environment), and/or some other computing devices capable of interfacing directly or indirectly with network 1199 or other network(s). For purposes of the present disclosure, the compute node 1100 may represent any of the computing devices discussed herein, and may be, or be implemented in one or more of NFT/blockchain service 402, 800, client systems 501, 810, 860, 1150, servers 820, 830, 850, 1190, and/or any other devices or systems, such as any of those discussed herein.

The system 1100 includes physical hardware devices and software components capable of providing and/or accessing content and/or services to/from the remote system 1190. The system 1100 and/or the remote system 1190 can be implemented as any suitable computing system or other data processing apparatus usable to access and/or provide content/services from/to one another. As examples, the system 1100 and/or the remote system 1190 may comprise desktop computers, a work stations, laptop computers, mobile cellular phones (e.g., “smartphones”), tablet computers, portable media players, wearable computing devices, server computer systems, an aggregation of computing resources (e.g., in a cloud-based environment), or some other computing devices capable of interfacing directly or indirectly with network 1199 or other network. The system 1100 communicates with remote systems 1190, and vice versa, to obtain/serve content/services using any suitable communication protocol, such as any of those discussed herein.

The compute node 1100 includes one or more processors 1101 (also referred to as “processor circuitry 1101”). The processor circuitry 1101 includes circuitry capable of sequentially and/or automatically carrying out a sequence of arithmetic or logical operations, and recording, storing, and/or transferring digital data. Additionally or alternatively, the processor circuitry 1101 includes any device capable of executing or otherwise operating computer-executable instructions, such as program code, software modules, and/or functional processes. The processor circuitry 1101 includes various hardware elements or components such as, for example, a set of processor cores and one or more of on-chip or on-die memory or registers, cache and/or scratchpad memory, low drop-out voltage regulators (LDOs), interrupt controllers, serial interfaces such as SPI, I2C or universal programmable serial interface circuit, real time clock (RTC), timer-counters including interval and watchdog timers, general purpose I/O, memory card controllers such as secure digital/multi-media card (SD/MMC) or similar, interfaces, mobile industry processor interface (MIPI) interfaces and Joint Test Access Group (JTAG) test access ports. Some of these components, such as the on-chip or on-die memory or registers, cache and/or scratchpad memory, may be implemented using the same or similar devices as the memory circuitry 1103 discussed infra. The processor circuitry 1101 is also coupled with memory circuitry 1103 and storage circuitry 1104, and is configured to execute instructions stored in the memory/storage to enable various apps, OSs, or other software elements to run on the platform 1100. In particular, the processor circuitry 1101 is configured to operate app software (e.g., instructions 1101x, 1103x, 1104x) to provide one or more services to a user of the compute node 1100 and/or user(s) of remote systems/devices.

The processor circuitry 1101 can be embodied as, or otherwise include one or multiple central processing units (CPUs), application processors, graphics processing units (GPUs), RISC processors, Acorn RISC Machine (ARM) processors, complex instruction set computer (CISC) processors, DSPs, FPGAs, programmable logic devices (PLDs), ASICs, baseband processors, radio-frequency integrated circuits (RFICs), microprocessors or controllers, multi-core processors, multithreaded processors, ultra-low voltage processors, embedded processors, a specialized x-processing units (xPUs) or a data processing unit (DPUs) (e.g., Infrastructure Processing Unit (IPU), network processing unit (NPU), and the like), and/or any other processing devices or elements, or any combination thereof. In some implementations, the processor circuitry 1101 is embodied as one or more special-purpose processor(s)/controller(s) configured (or configurable) to operate according to the various implementations and other aspects discussed herein. Additionally or alternatively, the processor circuitry 1101 includes one or more hardware accelerators (e.g., same or similar to acceleration circuitry 1108), which can include microprocessors, programmable processing devices (e.g., FPGAs, ASICs, PLDs, DSPs. and/or the like), and/or the like. As examples, the processor circuitry 1102 may include Intel® Core™ based processor(s), MCU-class processor(s), Xeon® processor(s); Advanced Micro Devices (AMD) Zen® Core Architecture processor(s), such as Ryzen® or Epyc® processor(s), Accelerated Processing Units (APUs), MxGPUs, or the like; A, S, W, and T series processor(s) from Apple® Inc., Snapdragon™ or Centriq™ processor(s) from Qualcomm® Technologies, Inc., Texas Instruments, Inc.® Open Multimedia Applications Platform (OMAP)™ processor(s); Power Architecture processor(s) provided by the OpenPOWER® Foundation and/or IBM®, MIPS Warrior M-class, Warrior I-class, and Warrior P-class processor(s) provided by MIPS Technologies, Inc.; ARM Cortex-A, Cortex-R, and Cortex-M family of processor(s) as licensed from ARM Holdings, Ltd.; the ThunderX2® provided by Cavium™, Inc.; GeForce®, Tegra®, Titan X®, Tesla®, Shield®, and/or other like GPUs provided by Nvidia®; or the like. Other examples of the processor circuitry 1101 may be mentioned elsewhere in the present disclosure.

The compute node 1100 also includes non-transitory or transitory machine readable media 1102 (also referred to as “computer readable medium 1102” or “CRM 1102”), which may be embodied as, or otherwise include system memory 1103, storage 1104, and/or memory devices/elements of the processor 1101. Additionally or alternatively, the CRM 1102 can be embodied as any of the devices/technologies described for the memory 1103 and/or storage 1104.

The system memory 1103 (also referred to as “memory circuitry 1103”) includes one or more hardware elements/devices for storing data and/or instructions 1103x (and/or instructions 1101x, 1104x). Any number of memory devices may be used to provide for a given amount of system memory 1103. As examples, the memory 1103 can be embodied as processor cache or scratchpad memory, volatile memory, non-volatile memory (NVM), and/or any other machine readable media for storing data. Examples of volatile memory include random access memory (RAM), static RAM (SRAM), dynamic RAM (DRAM), synchronous DRAM (SDRAM), thyristor RAM (T-RAM), content-addressable memory (CAM), and/or the like. Examples of NVM can include read-only memory (ROM) (e.g., including programmable ROM (PROM), erasable PROM (EPROM), electrically EPROM (EEPROM), flash memory (e.g., NAND flash memory, NOR flash memory, and the like), solid-state storage (SSS) or solid-state ROM, programmable metallization cell (PMC), and/or the like), non-volatile RAM (NVRAM), phase change memory (PCM) or phase change RAM (PRAM) (e.g., Intel® 3D XPoint™ memory, chalcogenide RAM (CRAM), Interfacial Phase-Change Memory (IPCM), and the like), memistor devices, resistive memory or resistive RAM (ReRAM) (e.g., memristor devices, metal oxide-based ReRAM, quantum dot resistive memory devices, and the like), conductive bridging RAM (or PMC), magnetoresistive RAM electrochemical RAM (MRAM), (ECRAM), ferroelectric RAM (FeRAM), anti-ferroelectric RAM (AFeRAM), ferroelectric field-effect transistor (FeFET) memory, and/or the like. Additionally or alternatively, the memory circuitry 1103 can include spintronic memory devices (e.g., domain wall memory (DWM), spin transfer torque (STT) memory (e.g., STT-RAM or STT-MRAM), magnetic tunneling junction memory devices, spin-orbit transfer memory devices, Spin-Hall memory devices, nanowire memory cells, and/or the like). In some implementations, the individual memory devices 1103 may be formed into any number of different package types, such as single die package (SDP), dual die package (DDP), quad die package (Q17P), memory modules (e.g., dual inline memory modules (DIMMs), microDIMMs, and/or MiniDIMMs), and/or the like. Additionally or alternatively, the memory circuitry 1103 is or includes block addressable memory device(s), such as those based on NAND or NOR flash memory technologies (e.g., single-level cell (“SLC”), multi-level cell (“MLC”), quad-level cell (“QLC”), tri-level cell (“TLC”), or some other NAND or NOR device). Additionally or alternatively, the memory circuitry 1103 can include resistor-based and/or transistor-less memory architectures. In some examples, the memory circuitry 1103 can refer to a die, chip, and/or a packaged memory product. In some implementations, the memory 1103 can be or include the on-die memory or registers associated with the processor circuitry 1101. Additionally or alternatively, the memory 1103 can include any of the devices/components discussed infra w.r.t the storage circuitry 1104.

The storage 1104 (also referred to as “storage circuitry 1104”) provides persistent storage of information, such as data, OSs, apps, instructions 1104x, and/or other software elements. As examples, the storage 1104 may be embodied as a magnetic disk storage device, hard disk drive (HDD), microHDD, solid-state drive (SSD), optical storage device, flash memory devices, memory card (e.g., secure digital (SD) card, extreme Digital (XD) picture card, USB flash drives, SIM cards, and/or the like), and/or any combination thereof. The storage circuitry 1104 can also include specific storage units, such as storage devices and/or storage disks that include optical disks (e.g., DVDs, CDs/CD-ROM, Blu-ray disks, and the like), flash drives, floppy disks, hard drives, and/or any number of other hardware devices in which information is stored for any duration (e.g., for extended time periods, permanently, for brief instances, for temporarily buffering, and/or caching). Additionally or alternatively, the storage circuitry 1104 can include resistor-based and/or transistor-less memory architectures. Further, any number of technologies may be used for the storage 1104 in addition to, or instead of, the previously described technologies, such as, for example, resistance change memories, phase change memories, holographic memories, chemical memories, among many others. Additionally or alternatively, the storage circuitry 1104 can include any of the devices or components discussed previously w.r.t the memory 1103.

Instructions 1101x, 1103x, 1104x in the form of computer programs, computational logic/modules (e.g., including the various modules/logic discussed herein), source code, middleware, firmware, object code, machine code, microcode (ucode), or hardware commands/instructions, when executed, implement or otherwise carry out various functions, processes, methods, algorithms, operations, tasks, actions, techniques, and/or other aspects of the present disclosure. The instructions 1101x, 1103x, 1104x may be written in any combination of one or more programming languages, including object oriented programming languages, procedural programming languages, scripting languages, markup languages, machine language, and/or some other suitable programming languages including proprietary programming languages and/or development tools, or any other suitable technologies. The instructions 1101x, 1103x, 1104x may execute entirely on the system 1100, partly on the system 1100, as a stand-alone software package, partly on the system 1100 and partly on a remote system 1190, or entirely on the remote system 1190. In the latter scenario, the remote system 1190 may be connected to the system 1100 through any type of network 1199. Although the instructions 1101x, 1103x, 1104x are shown as code blocks included in the processor 1101, memory 1103, and/or storage 1104, any of the code blocks may be replaced with hardwired circuits, for example, built into memory blocks/cells of an ASIC, FPGA, and/or some other suitable IC.

In some examples, the storage circuitry 1104 stores computational logic/modules configured to implement the techniques described herein. The computational logic 1104x may be employed to store working copies and/or permanent copies of programming instructions, or data to create the programming instructions, for the operation of various components of compute node 110, an OS of compute node 1100, one or more applications, and/or the like. The computational logic 1104x may be stored or loaded into memory circuitry 1103 as instructions 1103x, or data to create the instructions 1103x, which are then accessed for execution by the processor circuitry 1101 via the IX 1106 to carry out the various functions, processes, methods, algorithms, operations, tasks, actions, techniques, and/or other aspects described herein (see e.g., FIGS. 1-10). The various elements may be implemented by assembler instructions supported by processor circuitry 1101 or high-level languages that may be compiled into instructions 1101x, or data to create the instructions 1101x, to be executed by the processor circuitry 1101. The permanent copy of the programming instructions may be placed into persistent storage circuitry 1104 at the factory/OEM or in the field through, for example, a distribution medium (e.g., a wired connection and/or over-the-air (OTA) interface) and a communication interface (e.g., communication circuitry 1107) from a distribution server (e.g., remote system 1190) and/or the like.

Additionally or alternatively, the instructions 1101x, 1103x, 1104x can include one or more operating systems (OS) and/or other software to control various aspects of the compute node 1100. The OS can include drivers, libraries, APIs, firmware, middleware, and/or other interface or connection mechanisms to control particular devices or components that are embedded in the compute node 1100, attached to the compute node 1100, communicatively coupled with the compute node 1100, and/or otherwise accessible by the compute node 1100. The OSs also include one or more libraries, drivers, APIs, inter-process communications (IPC), facilities, firmware, middleware, software glue, software connector(s), and/or other interface or connection mechanisms, which provide program code and/or software components for one or more apps to obtain and use the data from other apps operated by the compute node 1100, such as the various subsystems of the NFT/blockchain service 402, 800 and/or any other device or system discussed herein. For example, the OS can include a display driver to control and allow access to a display device, a touchscreen driver to control and allow access to a touchscreen interface of the system 1100, sensor drivers to obtain sensor readings of sensor circuitry 1121 and control and allow access to sensor circuitry 1121, actuator drivers to obtain actuator positions of the actuators 1122 and/or control and allow access to the actuators 1122, a camera driver to control and allow access to an embedded image capture device, audio drivers to control and allow access to one or more audio devices. The OS can be a general purpose OS or an OS specifically written for and tailored to the computing platform 1100. Example OSs include consumer-based OS (e.g., Microsoft® Windows® 10, Google® Android®, Apple® macOS®, Apple® IOS®, KaiOS™ provided by KaiOS Technologies Inc., Unix or a Unix-like OS such as Linux, Ubuntu, or the like), industry-focused OSs such as real-time OS (RTOS) (e.g., Apache® Mynewt, Windows® IoT®, Android Things®, Micrium® Micro-Controller OSs (“MicroC/OS” or “μC/OS”), VxWorks®, FreeRTOS, and/or the like), hypervisors (e.g., Xen® Hypervisor, Real-Time Systems® RTS Hypervisor, Wind River Hypervisor, VMWare® vSphere® Hypervisor, and/or the like), and/or the like. For purposes of the present disclosure, can also include hypervisors, container orchestrators and/or container engines. The OS can invoke alternate software to facilitate one or more functions and/or operations that are not native to the OS, such as particular communication protocols and/or interpreters. Additionally or alternatively, the OS instantiates various functionalities that are not native to the OS. In some examples, OSs include varying degrees of complexity and/or capabilities. In some examples, a first OS on a first compute node 1100 may be the same or different than a second OS on a second compute node 1100 (here, the first and second compute nodes 1100 can be physical machines or VMs operating on the same or different physical compute nodes). In these examples, the first OS may be an RTOS having particular performance expectations of responsivity to dynamic input conditions, and the second OS can include GUI capabilities to facilitate end-user I/O and the like.

The various components of the computing node 1100 communicate with one another over an interconnect (IX) 1106. The IX 1106 may include any number of IX (or similar) technologies including, for example, instruction set architecture (ISA), extended ISA (eISA), Inter-Integrated Circuit (I2C), serial peripheral interface (SPI), point-to-point interfaces, power management bus (PMBus), peripheral component interconnect (PCI), PCI express (PCIe), PCI extended (PCIx), Intel® Ultra Path Interconnect (UPI), Intel® Accelerator Link, Intel® QuickPath Interconnect (QPI), Intel® Omni-Path Architecture (OPA), Compute Express Link™ (CXL™) IX, RapidIO™ IX, Coherent Accelerator Processor Interface (CAPI), OpenCAPI, Advanced Microcontroller Bus Architecture (AMBA) IX, cache coherent interconnect for accelerators (CCIX), Gen-Z Consortium IXs, a HyperTransport IX, NVLink provided by NVIDIA®, ARM Advanced extensible Interface (AXI), a Time-Trigger Protocol (TTP) system, a FlexRay system, PROFIBUS, Ethernet, USB, On-Chip System Fabric (IOSF), Infinity Fabric (IF), and/or any number of other IX technologies. The IX 1106 may be a proprietary bus, for example, used in a SoC based system.

In some implementations (e.g., where the system 1100 is a server computer system), the compute node 1100 includes one or more hardware accelerators 1110 (also referred to as “acceleration circuitry 1110”, “accelerator circuitry 1110”, or the like). The acceleration circuitry 1110 can include various hardware elements such as, for example, one or more GPUs, FPGAs, DSPs, SoCs (including programmable SoCs and multi-processor SoCs), ASICs (including programmable ASICs), PLDs (including complex PLDs (CPLDs) and high capacity PLDs (HCPLDs), xPUs (e.g., DPUs, IPUs, and NPUs) and/or other forms of specialized circuitry designed to accomplish specialized tasks. Additionally or alternatively, the acceleration circuitry 1110 may be embodied as, or include, one or more of AI/ML accelerators (e.g., vision processing unit (VPU), neural compute sticks, neuromorphic hardware, deep learning processors (DLPs) or deep learning accelerators, tensor processing units (TPUs), physical neural network hardware, and/or the like), cryptographic accelerators (or secure cryptoprocessors), network processors, I/O accelerator (e.g., DMA engines and the like), and/or any other specialized hardware device/component. The offloaded tasks performed by the acceleration circuitry 1110 can include, for example, AI/ML tasks (e.g., training, feature extraction, model execution for inference/prediction, classification, and so forth), visual data processing, graphics processing, digital and/or analog signal processing, network data processing, infrastructure function management, object detection, rule analysis, and/or the like. As examples, the processor(s) 1101 and/or accelerators 1110 may be a cluster of GPUs, tensor processing units (TPUs) developed by Google® Inc., Real AI Processors (RAPS™) provided by AlphaICs®, Nervana™ Neural Network Processors (NNPs) provided by Intel® Corp., Intel® Movidius™ Myriad™ X Vision Processing Unit (VPU), NVIDIA® PX™ based GPUs, the NM500 chip provided by General Vision®, Hardware 3 provided by Tesla®, Inc., an Epiphany™ based processor provided by Adapteva®, or the like. In some embodiments, the processor circuitry 1101 and/or hardware accelerator circuitry may be implemented as AI accelerating co-processor(s), such as the Hexagon 685 DSP provided by Qualcomm®, the PowerVR 2NX Neural Net Accelerator (NNA) provided by Imagination Technologies Limited®, the Neural Engine core within the Apple® A11 or A12 Bionic SoC, the Neural Processing Unit (NPU) within the HiSilicon Kirin 970 provided by Huawei®, and/or the like.

The acceleration circuitry 1110 includes any suitable hardware device or collection of hardware elements that are designed to perform one or more specific functions more efficiently in comparison to general-purpose processing elements (e.g., those provided as part of the processor circuitry 1101). For example, the acceleration circuitry 1110 can include special-purpose processing device tailored to perform one or more specific tasks or workloads of the subsystems of the NFT/blockchain service 402, 800. In some examples, the specific tasks or workloads may be offloaded from one or more processors of the processor circuitry 1101. In some implementations, the processor circuitry 1101 and/or acceleration circuitry 1110 includes hardware elements specifically tailored for executing, operating, or otherwise providing AI and/or ML functionality, such as for operating various subsystems of the NFT/blockchain service 402, 800 and/or any other device or system discussed previously with regard to FIGS. 1-10. In these implementations, the circuitry 1101 and/or 1110 is/are embodied as, or otherwise includes, one or more AI or ML chips that can run many different kinds of AI/ML instruction sets once loaded with the appropriate weightings, training data, AI/ML models, and/or the like. Additionally or alternatively, the processor circuitry 1101 and/or accelerator circuitry 1110 is/are embodied as, or otherwise includes, one or more custom-designed silicon cores specifically designed to operate corresponding subsystems of the NFT/blockchain service 402, 800 and/or any other device or system discussed herein. These cores may be designed as synthesizable cores comprising hardware description language logic (e.g., register transfer logic, verilog, Very High Speed Integrated Circuit hardware description language (VHDL), and the like); netlist cores comprising gate-level description of electronic components and connections and/or process-specific very-large-scale integration (VLSI) layout; and/or analog or digital logic in transistor-layout format. In these implementations, one or more of the subsystems of the NFT/blockchain service 402, 800 and/or any other device or system discussed herein may be operated, at least in part, on custom-designed silicon core(s). These “hardware-ized” subsystems may be integrated into a larger chipset but may be more efficient than using general purpose processor cores.

The TEE 1109 operates as a protected area accessible to the processor circuitry 1101 and/or other components to enable secure access to data and secure execution of instructions. The TEE 1109 operates as a protected area accessible to the processor circuitry 1101 to enable secure access to data and secure execution of instructions. In some implementations, the TEE 1109 is embodied as one or more physical hardware devices that is/are separate from other components of the system 1100, such as a secure-embedded controller, a dedicated SoC, a trusted platform module (TPM), a tamper-resistant chipset or microcontroller with embedded processing devices and memory devices, and/or the like. Examples of such implementations include a Desktop and mobile Architecture Hardware (DASH) compliant Network Interface Card (NIC), Intel® Management/Manageability Engine, Intel® Converged Security Engine (CSE) or a Converged Security Management/Manageability Engine (CSME), Trusted Execution Engine (TXE) provided by Intel® each of which may operate in conjunction with Intel® Active Management Technology (AMT) and/or Intel® vPro™ Technology; AMD® Platform Security coProcessor (PSP), AMD® PRO A-Series Accelerated Processing Unit (APU) with DASH manageability, Apple® Secure Enclave coprocessor; IBM® Crypto Express3®, IBM® 4807, 4808, 4809, and/or 4765 Cryptographic Coprocessors, IBM® Baseboard Management Controller (BMC) with Intelligent Platform Management Interface (IPMI), Dell™ Remote Assistant Card II (DRAC II), integrated Dell™ Remote Assistant Card (iDRAC), and the like.

Additionally or alternatively, the TEE 1109 is embodied as secure enclaves (or “enclaves”), which is/are isolated regions of code and/or data within the processor and/or memory/storage circuitry of the compute node 1100, where only code executed within a secure enclave may access data within the same secure enclave, and the secure enclave may only be accessible using the secure app (which may be implemented by an app processor or a tamper-resistant microcontroller). In some implementations, the memory circuitry 1103 and/or storage circuitry 1104 may be divided into one or more trusted memory regions for storing apps or software modules of the secure enclave(s) 1109. Example implementations of the TEE 1109, and an accompanying secure area in the processor circuitry 1101 or the memory circuitry 1103 and/or storage circuitry 1104, include Intel® Software Guard Extensions (SGX), ARM® TrustZone® hardware security extensions, Keystone Enclaves provided by Oasis Labs™, and/or the like. Other aspects of security hardening, hardware roots-of-trust, and trusted or protected operations may be implemented in the device 1100 through the TEE 1109 and the processor circuitry 1101.

Additionally or alternatively, the TEE 1109 and/or processor circuitry 1101, acceleration circuitry 1110, memory circuitry 1103, and/or storage circuitry 1104 may be divided into, or otherwise separated into isolated user-space instances and/or virtualized environments using a suitable virtualization technology, such as, for example, virtual machines (VMs), virtualization containers (e.g., Docker® containers, Kubernetes® containers, Solaris® containers and/or zones, OpenVZ® virtual private servers, DragonFly BSD® virtual kernels and/or jails, chroot jails, and/or the like), and/or other virtualization technologies. These virtualization technologies may be managed and/or controlled by a virtual machine monitor (VMM), hypervisor container engines, orchestrators, and the like. Such virtualization technologies provide execution environments/TEEs in which one or more apps and/or other software, code, or scripts may execute while being isolated from one or more other apps, software, code, or scripts.

The communication circuitry 1107 is a hardware element, or collection of hardware elements, used to communicate over one or more networks (e.g., network 1199) and/or with other devices. The communication circuitry 1107 includes modem 1107a and transceiver circuitry (“TRx”) 1107b. The modem 1107a includes one or more processing devices (e.g., baseband processors) to carry out various protocol and radio control functions. Modem 1107a may interface with application circuitry of compute node 1100 (e.g., a combination of processor circuitry 1101, memory circuitry 1103, and/or storage circuitry 1104) for generation and processing of baseband signals and for controlling operations of the TRx 1107b. The modem 1107a handles various radio control functions that enable communication with one or more radio networks via the TRx 1107b according to one or more wireless communication protocols. The modem 1107a may include circuitry such as, but not limited to, one or more single-core or multi-core processors (e.g., one or more baseband processors) or control logic to process baseband signals received from a receive signal path of the TRx 1107b, and to generate baseband signals to be provided to the TRx 1107b via a transmit signal path. In various implementations, the modem 1107a may implement a real-time OS (RTOS) to manage resources of the modem 1107a, schedule tasks, and the like.

The communication circuitry 1107 also includes TRx 1107b to enable communication with wireless networks using modulated electromagnetic radiation through a non-solid medium. The TRx 1107b may include one or more radios that are compatible with, and/or may operate according to any one or more of the radio communication technologies, radio access technologies (RATs), and/or communication protocols/standards including any combination of those discussed herein. TRx 1107b includes a receive signal path, which comprises circuitry to convert analog RF signals (e.g., an existing or received modulated waveform) into digital baseband signals to be provided to the modem 1107a. The TRx 1107b also includes a transmit signal path, which comprises circuitry configured to convert digital baseband signals provided by the modem 1107a to be converted into analog RF signals (e.g., modulated waveform) that will be amplified and transmitted via an antenna array including one or more antenna elements (not shown). The antenna array may be a plurality of microstrip antennas or printed antennas that are fabricated on the surface of one or more printed circuit boards. The antenna array may be formed in as a patch of metal foil (e.g., a patch antenna) in a variety of shapes, and may be coupled with the TRx 1107b using metal transmission lines or the like.

The network interface circuitry/controller (NIC) 1107c provides wired communication to the network 1199 and/or to other devices using an access technology and/or communication protocol such as, for example, Ethernet (e.g., IEEE 802.3), Ethernet over GRE Tunnels, Ethernet over Multiprotocol Label Switching (MPLS), Ethernet over USB, Controller Area Network (CAN), Local Interconnect Network (LIN), DeviceNet, ControlNet, Data Highway+, PROFIBUS, or PROFINET, among many others, including any of those mentioned herein and/or in '074 and/or '081. Network connectivity may be provided to/from the compute node 1100 via the NIC 1107c using a physical connection, which may be electrical (e.g., a “copper interconnect”), fiber, and/or optical. The physical connection also includes suitable input connectors (e.g., ports, receptacles, sockets, and the like) and output connectors (e.g., plugs, pins, and the like). The NIC 1107c may include one or more dedicated processors and/or FPGAs to communicate using one or more of the aforementioned network interface protocols. As examples, the NIC 1107c is embodied as an Ethernet controller (e.g., a Gigabit Ethernet Controller or the like), a high-speed serial interface (HSSI), a Peripheral Component Interconnect (PCI) controller, a USB controller, a SmartNIC, an Intelligent Fabric Processor (IFP), and/or some other like device. In some implementations, the NIC 1107c may include multiple controllers to provide connectivity to other networks using the same or different protocols. For example, the compute node 1100 may include a first NIC 1107c providing communications to the network 1199 over Ethernet and a second NIC 1107c providing communications to other devices over another type of network.

The interface circuitry 1108 (also referred to as “input/output (I/O) interface circuitry 1108”, “I/O circuitry 1108”, and/or the like) is configured to connect or communicatively coupled the compute node 1100 with one or more external (peripheral) components, devices, and/or subsystems. Examples of such external (peripheral) components/devices include sensor circuitry 1141, actuator circuitry 1142, positioning circuitry 1143, and other I/O devices 1140, but may also include other devices or subsystems not shown by FIG. 11. In some implementations, the interface circuitry 1108, is part of, or includes circuitry that enables the exchange of information between two or more components or devices internal to the compute node 1100. Additionally or alternatively, the interface circuitry 1108 may be used to transfer data between the compute node 1100 and another computer device (e.g., remote system 1190, client system 1150, and/or the like) via a wired and/or wireless connection. Access to various such devices/components may be implementation specific, and may vary from implementation to implementation. As examples, the interface circuitry 1108 can be embodied as, or otherwise include, one or more hardware interfaces such as, for example, buses (e.g., including an expansion buses, IXs, and/or the like), input/output (I/O) interfaces, peripheral component interfaces (e.g., peripheral cards and/or the like), network interface cards, host bus adapters, and/or mezzanines, and/or the like. In some implementations, the interface circuitry 1108 includes one or more interface controllers and connectors that interconnect one or more of the processor circuitry 1101, memory circuitry 1103, storage circuitry 1104, communication circuitry 1107, and the other components of compute node 1100 and/or to one or more external (peripheral) components, devices, and/or subsystems.

Additionally or alternatively, the interface circuitry 1108 and/or the IX 1106 can be embodied as, or otherwise include memory controllers, storage controllers (e.g., redundant array of independent disk (RAID) controllers and the like), baseboard management controllers (BMCs), input/output (I/O) controllers, host controllers, and the like. Examples of I/O controllers include integrated memory controller (IMC), memory management unit (MMU), input-output MMU (IOMMU), sensor hub, General Purpose I/O (GPIO) controller, PCIe endpoint (EP) device, direct media interface (DMI) controller, Intel® Flexible Display Interface (FDI) controller(s), VGA interface controller(s), Peripheral Component Interconnect Express (PCIe) controller(s), universal serial bus (USB) controller(s), FireWire controller(s), Thunderbolt controller(s), FPGA Mezzanine Card (FMC), extensible Host Controller Interface (xHCI) controller(s), Enhanced Host Controller Interface (EHCI) controller(s), Serial Peripheral Interface (SPI) controller(s), Direct Memory Access (DMA) controller(s), hard drive controllers (e.g., Serial AT Attachment (SATA) host bus adapters/controllers, Intel® Rapid Storage Technology (RST), and/or the like), Advanced Host Controller Interface (AHCI), a Low Pin Count (LPC) interface (bridge function), Advanced Programmable Interrupt Controller(s) (APIC), audio controller(s), SMBus host interface controller(s), UART controller(s), and/or the like. Some of these controllers may be part of, or otherwise applicable to the memory circuitry 1103, storage circuitry 1104, and/or IX 1106 as well. As examples, the connectors include electrical connectors, ports, slots, jumpers, receptacles, modular connectors, coaxial cable and/or BNC connectors, optical fiber connectors, PCB mount connectors, inline/cable connectors, chassis/panel connectors, peripheral component interfaces (e.g., non-volatile memory ports, USB ports, Ethernet ports, audio jacks, power supply interfaces, on-board diagnostic (OBD) ports, and so forth), and/or the like.

The sensor(s) 1141 (also referred to as “sensor circuitry 1141”) includes devices, modules, or subsystems whose purpose is to detect events or changes in its environment and send the information (sensor data) about the detected events to some other a device, module, subsystem, and the like. Individual sensors 1141 may be exteroceptive sensors (e.g., sensors that capture and/or measure environmental phenomena and/external states), proprioceptive sensors (e.g., sensors that capture and/or measure internal states of the compute node 1100 and/or individual components of the compute node 1100), and/or exproprioceptive sensors (e.g., sensors that capture, measure, or correlate internal states and external states). Examples of such sensors 1141 include inertia measurement units (IMU), microelectromechanical systems (MEMS) or nanoelectromechanical systems (NEMS), level sensors, flow sensors, temperature sensors (e.g., thermistors, including sensors for measuring the temperature of internal components and sensors for measuring temperature external to the compute node 1100), pressure sensors, barometric pressure sensors, gravimeters, altimeters, image capture devices (e.g., visible light cameras, thermographic camera and/or thermal imaging camera (TIC) systems, forward-looking infrared (FLIR) camera systems, radiometric thermal camera systems, active infrared (IR) camera systems, ultraviolet (UV) camera systems, and/or the like), light detection and ranging (LiDAR) sensors, proximity sensors (e.g., IR radiation detector and the like), depth sensors, ambient light sensors, optical light sensors, ultrasonic transceivers, microphones, inductive loops, force and/or load sensors, remote charge converters (RCC), rotor speed and position sensor(s), fiber optic gyro (FOG) inertial sensors, Attitude & Heading Reference Unit (AHRU), fibre Bragg grating (FBG) sensors and interrogators, tachometers, engine temperature gauges, pressure gauges, transformer sensors, airspeed-measurement meters, speed indicators, and/or the like. The IMUs, MEMS, and/or NEMS can include, for example, one or more 3-axis accelerometers, one or more 3-axis gyroscopes, one or more magnetometers, one or more compasses, one or more barometers, and/or the like. Additionally or alternatively, the sensors 1141 can include sensors of various compute components such as, for example, digital thermal sensors (DTS) of respective processors/cores, thermal sensor on-die (TSOD) of respective dual inline memory modules (DIMMs), baseboard thermal sensors, and/or any other sensor(s), such as any of those discussed herein.

The actuators 1142 allow the compute node 1100 to change its state, position, and/or orientation, or move or control a mechanism or system. The actuators 1142 comprise electrical and/or mechanical devices for moving or controlling a mechanism or system, and converts energy (e.g., electric current or moving air and/or liquid) into some kind of motion. The compute node 1100 is configured to operate one or more actuators 1142 based on one or more captured events, instructions, control signals, and/or configurations received from a service provider 1190, client device 1150, and/or other components of the compute node 1100. As examples, the actuators 1142 can be or include any number and combination of the following: soft actuators (e.g., actuators that changes its shape in response to a stimuli such as, for example, mechanical, thermal, magnetic, and/or electrical stimuli), hydraulic actuators, pneumatic actuators, mechanical actuators, electromechanical actuators (EMAs), microelectromechanical actuators, electrohydraulic actuators, linear actuators, linear motors, rotary motors, DC motors, stepper motors, servomechanisms, electromechanical switches, electromechanical relays (EMRs), power switches, valve actuators, piezoelectric actuators and/or biomorphs, thermal biomorphs, solid state actuators, solid state relays (SSRs), shape-memory alloy-based actuators, electroactive polymer-based actuators, relay driver integrated circuits (ICs), solenoids, impactive actuators/mechanisms (e.g., jaws, claws, tweezers, clamps, hooks, mechanical fingers, humaniform dexterous robotic hands, and/or other gripper mechanisms that physically grasp by direct impact upon an object), propulsion actuators/mechanisms (e.g., wheels, axles, thrusters, propellers, engines, motors (e.g., those discussed previously), clutches, and the like), projectile actuators/mechanisms (e.g., mechanisms that shoot or propel objects or elements), controllers of the compute node 1100 or components thereof (e.g., host controllers, cooling element controllers, baseboard management controller (BMC), platform controller hub (PCH), uncore components (e.g., shared last level cache (LLC) cache, caching agent (Cbo), integrated memory controller (IMC), home agent (HA), power control unit (PCU), configuration agent (Ubox), integrated I/O controller (IIO), and interconnect (IX) link interfaces and/or controllers), and/or any other components such as any of those discussed herein), audible sound generators, visual warning devices, virtual instrumentation and/or virtualized actuator devices, and/or other like components or devices. In some examples, such as when the compute node 1100 is part of a robot or drone, the actuator(s) 1142 can be embodied as or otherwise represent one or more end effector tools, conveyor motors, and/or the like.

The positioning circuitry 1143 includes circuitry to receive and decode signals transmitted/broadcasted by a positioning network of a GNSS. Examples of such navigation satellite constellations include United States' GPS, Russia's Global Navigation System (GLONASS), the European Union's Galileo system, China's BeiDou Navigation Satellite System, a regional navigation system or GNSS augmentation system (e.g., Navigation with Indian Constellation (NAVIC), Japan's Quasi-Zenith Satellite System (QZSS), France's Doppler Orbitography and Radio-positioning Integrated by Satellite (DORIS), and the like), or the like. The positioning circuitry 1143 comprises various hardware elements (e.g., including hardware devices such as switches, filters, amplifiers, antenna elements, and the like to facilitate OTA communications) to communicate with components of a positioning network, such as navigation satellite constellation nodes. In some implementations, the positioning circuitry 1143 may include a Micro-Technology for Positioning, Navigation, and Timing (Micro-PNT) IC that uses a master timing clock to perform position tracking/estimation without GNSS assistance. The positioning circuitry 1143 may also be part of, or interact with, the communication circuitry 1107 to communicate with the nodes and components of the positioning network. The positioning circuitry 1143 may also provide position data and/or time data to the application circuitry, which may use the data to synchronize operations with various infrastructure (e.g., radio base stations), for turn-by-turn navigation, or the like.

NFC circuitry 1146 comprises one or more hardware devices and software modules configurable or operable to read electronic tags and/or connect with another NFC-enabled device (also referred to as an “NFC touchpoint”). NFC is commonly used for contactless, short-range communications based on radio frequency identification (RFID) standards, where magnetic field induction is used to enable communication between NFC-enabled devices. The one or more hardware devices may include an NFC controller coupled with an antenna element and a processor coupled with the NFC controller. The NFC controller may be a chip providing NFC functionalities to the NFC circuitry 1146. The software modules may include NFC controller firmware and an NFC stack. The NFC stack may be executed by the processor to control the NFC controller, and the NFC controller firmware may be executed by the NFC controller to control the antenna element to emit an RF signal. The RF signal may power a passive NFC tag (e.g., a microchip embedded in a sticker or wristband) to transmit stored data to the NFC circuitry 1146, or initiate data transfer between the NFC circuitry 1146 and another active NFC device (e.g., a smartphone or an NFC-enabled point-of-sale terminal) that is proximate to the computing system 1100 (or the NFC circuitry 1146 contained therein). The NFC circuitry 1146 may include other elements, such as those discussed herein. Additionally, the NFC circuitry 1146 may interface with a secure element (e.g., TEE 1109) to obtain payment credentials and/or other sensitive/secure data to be provided to the other active NFC device. Additionally or alternatively, the NFC circuitry 1146 and/or some other element may provide Host Card Emulation (HCE), which emulates a physical secure element.

The I/O device(s) 1140 may be present within, or connected to, the compute node 1100. The I/O devices 1140 include input device circuitry and output device circuitry including one or more user interfaces designed to enable user interaction with the compute node 1100 and/or peripheral component interfaces designed to enable peripheral component interaction with the compute node 1100. The input device circuitry includes any physical or virtual means for accepting an input including, inter alia, one or more physical or virtual buttons, a physical or virtual keyboard, keypad, mouse, touchpad, touchscreen, microphones, scanner, headset, and/or the like. In implementations where the input device circuitry includes a capacitive, resistive, or other like touch-surface, a touch signal may be obtained from circuitry of the touch-surface. The touch signal may include information regarding a location of the touch (e.g., one or more sets of (x,y) coordinates describing an area, shape, and/or movement of the touch), a pressure of the touch (e.g., as measured by area of contact between a user's finger or a deformable stylus and the touch-surface, or by a pressure sensor), a duration of contact, any other suitable information, or any combination of such information. In these implementations, one or more apps operated by the processor circuitry 1101 may identify gesture(s) based on the information of the touch signal, and utilizing a gesture library that maps determined gestures with specified actions.

The output device circuitry is used to show or convey information, such as sensor readings, actuator position(s), or other like information. Data and/or graphics may be displayed on one or more user interface components of the output device circuitry. The output device circuitry may include any number and/or combinations of audio or visual display, including, inter alia, one or more simple visual outputs/indicators (e.g., binary status indicators (e.g., light emitting diodes (LEDs)) and multi-character visual outputs, or more complex outputs such as display devices or touchscreens (e.g., Liquid Chrystal Displays (LCD), LED and/or OLED displays, quantum dot displays, projectors, and the like), with the output of characters, graphics, multimedia objects, and the like being generated or produced from operation of the compute node 1100. The output device circuitry may also include speakers or other audio emitting devices, printer(s), and/or the like. In some implementations, the sensor circuitry 1141 may be used as the input device circuitry (e.g., an image capture device, motion capture device, or the like) and one or more actuators 1142 may be used as the output device circuitry (e.g., an actuator to provide haptic feedback or the like). In another example, near-field communication (NFC) circuitry comprising an NFC controller coupled with an antenna element and a processing device may be included to read electronic tags and/or connect with another NFC-enabled device. Peripheral component interfaces may include, but are not limited to, a non-volatile memory port, a universal serial bus (USB) port, an audio jack, a power supply interface, and the like.

A battery 1114 may be coupled to the compute node 1100 to power the compute node 1100, which may be used in implementations where the compute node 1100 is not in a fixed location, such as when the compute node 1100 is a mobile device or laptop. The battery 1114 may be a lithium ion battery, a lead-acid automotive battery, or a metal-air battery, such as a zinc-air battery, an aluminum-air battery, a lithium-air battery, a lithium polymer battery, and/or the like. In implementations where the compute node 1100 is mounted in a fixed location, such as when the system is implemented as a server computer system, the compute node 1100 may have a power supply coupled to an electrical grid. In these implementations, the compute node 1100 may include power tee circuitry to provide for electrical power drawn from a network cable to provide both power supply and data connectivity to the compute node 1100 using a single cable.

Power management integrated circuitry (PMIC) 1122 may be included in the compute node 1100 to track the state of charge (SoCh) of the battery 1114, and to control charging of the compute node 1100. The PMIC 1122 may be used to monitor other parameters of the battery 1114 to provide failure predictions, such as the state of health (SoH) and the state of function (SoF) of the battery 1114. The PMIC 1122 may include voltage regulators, surge protectors, power alarm detection circuitry. The power alarm detection circuitry may detect one or more of brown out (under-voltage) and surge (over-voltage) conditions. The PMIC 1122 may communicate the information on the battery 1114 to the processor circuitry 1101 over the IX 1106. The PMIC 1122 may also include an analog-to-digital (ADC) convertor that allows the processor circuitry 1101 to directly monitor the voltage of the battery 1114 or the current flow from the battery 1114. The battery parameters may be used to determine actions that the compute node 1100 may perform, such as transmission frequency, mesh network operation, sensing frequency, and the like.

A power block 1120, or other power supply coupled to an electrical grid, may be coupled with the PMIC 1122 to charge the battery 1114. In some examples, the power block 1120 may be replaced with a wireless power receiver to obtain the power wirelessly, for example, through a loop antenna in the compute node 1100. In these implementations, a wireless battery charging circuit may be included in the PMIC 1122. The specific charging circuits chosen depend on the size of the battery 1114 and the current required.

The compute node 1100 may include any combinations of the components shown by FIG. 11; however, some of the components shown may be omitted, additional components may be present, and different arrangement of the components shown may occur in other implementations. In one example where the compute node 1100 is or is part of a server computer system, the battery 1114, communication circuitry 1107, the sensors 1141, actuators 1142, and/or positioning circuitry 1143, and possibly some or all of the I/O devices 1140, may be omitted.

As mentioned previously, the memory circuitry 1103 and/or the storage circuitry 1104 are embodied as transitory or non-transitory computer-readable media (e.g., CRM 1102). The CRM 1102 is suitable for use to store instructions (or data that creates the instructions) that cause an apparatus (such as any of the devices/components/systems described w.r.t FIGS. 1-10), in response to execution of the instructions (e.g., instructions 1101x, 1103x, 1104x) by the compute node 1100 (e.g., one or more processors 1101), to practice selected aspects of the present disclosure. The CRM 1102 can include a number of programming instructions (e.g., instructions 1101x, 1103x, 1104x) (or data to create the programming instructions). The programming instructions are configured to enable a device (e.g., any of the devices/components/systems described w.r.t FIGS. 1-13), in response to execution of the programming instructions, to perform various programming operations associated with operating system functions, one or more apps, and/or aspects of the present disclosure (including various programming operations associated with FIGS. 1-11). The programming instructions may correspond to any of the computational logic 1104x, instructions 1103x and 1101x discussed previously.

Additionally or alternatively, programming instructions (or data to create the instructions) may be disposed on multiple CRM 1102. In alternate implementations, programming instructions (or data to create the instructions) may be disposed on computer-readable transitory storage media, such as signals. The programming instructions embodied by a machine readable medium 1102 may be transmitted or received over a communications network using a transmission medium via a network interface device (e.g., communication circuitry 1107 and/or NIC 1107c of FIG. 11) utilizing any one of a number of communication protocols and/or data transfer protocols such as any of those discussed herein.

Any combination of one or more computer usable or CRM 1102 may be utilized as or instead of the CRM 1102. The computer-usable or computer-readable medium 1102 may be, for example, but not limited to one or more electronic, magnetic, optical, electromagnetic, infrared, or semiconductor systems, apparatuses, devices, or propagation media. For instance, the CRM 1102 may be embodied by devices described for the storage circuitry 1104 and/or memory circuitry 1103 described previously and/or as discussed elsewhere in the present disclosure. In the context of the present disclosure, a computer-usable or computer-readable medium 1102 may be any medium that can contain, store, communicate, propagate, or transport the program (or data to create the program) for use by or in connection with the instruction execution system, apparatus, or device. The computer-usable medium 1102 may include a propagated data signal with the computer-usable program code (e.g., including programming instructions) or data to create the program code embodied therewith, either in baseband or as part of a carrier wave. The computer usable program code or data to create the program may be transmitted using any appropriate medium, including but not limited to wireless, wireline, optical fiber cable, RF, and the like.

Additionally or alternatively, the program code (or data to create the program code) described herein may be stored in one or more of a compressed format, an encrypted format, a fragmented format, a packaged format, and/or the like. Program code (e.g., programming instructions) or data to create the program code as described herein may require one or more of installation, modification, adaptation, updating, combining, supplementing, configuring, decryption, decompression, unpacking, distribution, reassignment, and the like in order to make them directly readable and/or executable by a computing device and/or other machine. For example, the program code or data to create the program code may be stored in multiple parts, which are individually compressed, encrypted, and stored on separate computing devices, wherein the parts when decrypted, decompressed, and combined form a set of executable instructions that implement the program code or the data to create the program code, such as those described herein. In another example, the program code or data to create the program code may be stored in a state in which they may be read by a computer, but require addition of a library (e.g., a dynamic link library), a software development kit (SDK), an API, and the like in order to execute the instructions on a particular computing device or other device. In another example, the program code or data to create the program code may need to be configured (e.g., settings stored, data input, network addresses recorded, and the like) before the program code or data to create the program code can be executed/used in whole or in part. In this example, the program code (or data to create the program code) may be unpacked, configured for proper execution, and stored in a first location with the configuration instructions located in a second location distinct from the first location. The configuration instructions can be initiated by an action, trigger, or instruction that is not co-located in storage or execution location with the instructions enabling the disclosed techniques. Accordingly, the disclosed program code or data to create the program code are intended to encompass such machine readable instructions and/or program(s) or data to create such machine readable instruction and/or programs regardless of the particular format or state of the machine readable instructions and/or program(s) when stored or otherwise at rest or in transit.

The computer program code for carrying out operations of the present disclosure, including, for example, programming instructions, computational logic 1104x, instructions 1103x, and/or instructions 1101x, may be written in any combination of one or more programming languages, including an object oriented programming language (e.g., Python, PyTorch, Ruby, Scala, Smalltalk, Java™, Java Servlets, Kotlin, C++, C#, and/or the like), a procedural programming language (e.g., the “C” programming language, Go (or “Golang”), and/or the like), a scripting language (e.g., ECMAScript, JavaScript, Server-Side JavaScript (SSJS), PHP, Pearl, Python, PyTorch, Ruby, Lua, Torch/Lua with Just-In Time compiler (LuaJIT), Accelerated Mobile Pages Script (AMPscript), VBScript, and/or the like), a markup language (e.g., hypertext markup language (HTML), extensible markup language (XML), wiki markup or Wikitext, User Interface Markup Language (UIML), and/or the like), a data interchange format/definition (e.g., Java Script Object Notion (JSON), Apache® MessagePack™, and/or the like), a stylesheet language (e.g., Cascading Stylesheets (CSS), extensible stylesheet language (XSL), and/or the like), an interface definition language (IDL) (e.g., Apache® Thrift, Abstract Syntax Notation One (ASN.1), Google® Protocol Buffers (protobuf), efficient XML interchange (EXI), and/or the like), a web framework (e.g., Active Server Pages Network Enabled Technologies (ASP.NET), Apache® Wicket, Asynchronous Javascript and XML (Ajax) frameworks, Django, Jakarta Server Faces (JSF; formerly JavaServer Faces), Jakarta Server Pages (JSP; formerly JavaServer Pages), Ruby on Rails, web toolkit, and/or the like), a template language (e.g., Apache® Velocity, Tea, Django template language, Mustache, Template Attribute Language (TAL), Extensible Stylesheet Language Transformations (XSLT), Thymeleaf, Facelet view, and/or the like), and/or some other suitable programming languages including proprietary programming languages and/or development tools, or any other languages or tools such as those discussed herein. It should be noted that some of the aforementioned languages, tools, and/or technologies may be classified as belonging to multiple types of languages/technologies or otherwise classified differently than described previously. The computer program code for carrying out operations of the present disclosure may also be written in any combination of the programming languages discussed herein. The program code may execute entirely on the compute node 1100, partly on the compute node 1100 as a stand-alone software package, partly on the compute node 1100 and partly on a remote computer, or entirely on the remote computer. In the latter scenario, the remote computer may be connected to the compute node 1100 through any type of network (e.g., network 1199).

The network 1199 comprises a set of computers that share resources located on or otherwise provided by a set of network nodes. The set of computers making up the network 1199 can use one or more communication protocols and/or access technologies (such as any of those discussed herein) to communicate with one another and/or with other computers outside of the network 1199 (e.g., compute node 1100, client device 1150, and/or remote system 1190), and may be connected with one another or otherwise arranged in a variety of network topologies. As examples, the network 1199 can represent the Internet, one or more cellular networks (e.g., 3GPP 5G, LTE, and/or the like), local area networks (LANs), wide area networks (WANs), wireless LANS (WLANs), Transfer Control Protocol (TCP)/Internet Protocol (IP)-based networks, Personal Area Networks (PANs), Digital Subscriber Line (DSL) and/or cable networks, data networks, cloud computing services, edge computing networks, proprietary and/or enterprise networks, virtual private network (VPN), and/or any combination thereof. In some implementations, the network 1199 is associated with network operator who owns or controls equipment and other elements necessary to provide network-related services, such as one or more network access nodes (NANs) (e.g., base stations, access points, and the like), one or more servers for routing digital data or telephone calls (e.g., a core network or backbone network), and the like. In either implementation, the network 1199 comprises computers, network connections among various computers (e.g., between the compute node 1100, client device(s) 1150, remote system 1190, and/or the like), and software routines to enable communication between the computers over respective network connections. Connections to the network 1199 (and/or compute nodes therein) may be via a wired and/or a wireless connections using the various communication protocols such as any of those discussed herein. More than one network may be involved in a communication session between the illustrated devices. Connection to the network 1199 may require that the computers execute software routines that enable, for example, the layers of the OSI model of computer networking or equivalent in a wireless (or cellular) phone network.

The remote system 1190 (also referred to as a “service provider”, “application server(s)”, “app server(s)”, “external platform”, and/or the like) comprises one or more physical and/or virtualized computing systems owned and/or operated by a company, enterprise, and/or individual that hosts, serves, and/or otherwise provides information objects to one or more users (e.g., compute node 1100). The physical and/or virtualized systems include one or more logically or physically connected servers and/or data storage devices distributed locally or across one or more geographic locations. Generally, the remote system 1190 uses IP/network resources to provide InOb(s) such as electronic documents, webpages, forms, apps (e.g., native apps, web apps, mobile apps, and/or the like), data, services, web services, media, and/or content to different user/client devices 1150. As examples, the service provider 1190 may provide mapping and/or navigation services; cloud computing services; search engine services; social networking, microblogging, and/or message board services; content (media) streaming services; e-commerce services; blockchain services; communication services such as Voice-over-Internet Protocol (VOIP) sessions, text messaging, group communication sessions, and the like; immersive gaming experiences; and/or other like services. In some examples, the remote system 1190 corresponds to the NFT/blockchain service 402, 800, and the system 1100 and/or client device 1150 corresponds to user system 501 and/or client devices 810, 860. The client devices that utilize services provided by remote system 1190 may be referred to as “subscribers” or the like. Although FIG. 11 shows only a single remote system 1190, the remote system 1190 may represent multiple remote system 1190, each of which may have their own subscribing users.

### 3. Example Implementations

Additional examples of the presently described method, system, and device embodiments include the following, non-limiting implementations. Each of the following non-limiting examples may stand on its own or may be combined in any permutation or combination with any one or more of the other examples provided below or throughout the present disclosure.

Example A01 includes a method of obtaining Non-Fungible Token (NFT)-based electronic health record (EHR) services, comprising: providing, via an application (app), user data to an NFT/blockchain service; and receiving, from the NFT/blockchain service via the app, a minted NFT based on the provided user data.

Example A02 includes the method of example A01 and/or some other example(s) herein, wherein the app is a client app, a wallet app, a payment app, a mobile app, a web app, a hybrid app, or a combination thereof. Example A03 includes the method of examples A01-A02 and/or some other example(s) herein, wherein the method is performed by a client computing device or a set of server computing devices.

Example A04 includes a method for providing Non-Fungible Token (NFT)-based electronic health record (EHR) services, comprising: receiving, by an NFT/blockchain service, inputs from one or more data sources, wherein at least some of the inputs include user data; generating, by the NFT/blockchain service, one or more smart contracts based on the received inputs; and minting, by the NFT/blockchain service, an NFT based on the received inputs and/or according to the generated smart contract.

Example A05 includes the method of example A04 and/or some other example(s) herein, wherein the method includes: receiving, by the NFT/blockchain service, the user data via a user application (app); generating, by the NFT/blockchain service, the minted NFT based on the provided user data; and sending the minted NFT to the user app.

Example A06 includes the method of examples A04-A05 and/or some other example(s) herein, wherein the method includes: generating a block for inclusion in a blockchain, wherein the block includes the minted NFT; and adding the generated block to the blockchain.

Example A07 includes the method of examples A04-A06 and/or some other example(s) herein, wherein the generated smart contract includes conditions, parameters, and/or criteria for detecting changes to EHR data.

Example A08 includes the method of examples A04-A07 and/or some other example(s) herein, wherein the generated smart contract includes conditions, parameters, and/or criteria for predicting or detecting diagnostic information based on EHR data.

Example A09 includes the method of examples A04-A08 and/or some other example(s) herein, wherein the method includes: receiving, by the NFT/blockchain service, EHR rules or policies from one or more healthcare providers (HPs); generating, by the NFT/blockchain service, a set of real-time codes from the EHR rules or policies; and generating, by the NFT/blockchain service, the smart contract based on the set of real-time codes.

Example A09 includes the method of example A08 and/or some other example(s) herein, wherein the EHR rules or policies include one or more healthcare-related rules, policies, and/or regulations. Example A10 includes the method of examples A01-A09 and/or some other example(s) herein, wherein the user data includes EHR data provided by a user or one or more HPs.

Example A11 includes the method of examples A01-A10 and/or some other example(s) herein, wherein the inputs include the user data and one or more of ID documents, financial institution documents, personal data, confidential data, sensitive data, authentication credentials, digital certificates, biometric data, one or more network addresses, one or more timestamps, geolocation information associated with access of the NFT/blockchain service, user ID, client app ID, app type of the app, version of the app, app session ID of the app, user agent string, operating system (OS) type, OS version, app vendor, OS vendor, network session ID, device ID or serial number, a product ID, EPC, RFID tag ID, an integer assigned to the user, a cookie ID, a realm name, domain name, logon user name or credentials, network credentials, social media account name, session ID, device fingerprint, knowledge-based authentication (KBA) data, know your customer (KYC) data, anti-money laundering (AML) data, and/or any other data or information such as those discussed herein.

Example A12 includes the method of examples A01-A11 and/or some other example(s) herein, wherein the method includes any one or more aspects as shown and described herein.

Example A13 includes the method of examples A01-A12 and/or some other example(s) herein, wherein the NFT/blockchain service is implemented by a set of servers, wherein the set of servers are part of a cloud computing service, an edge computing network, and/or a cellular communications network.

Example B01 includes a method for obtaining electronic health record (EHR) non-fungible token (NFT) services, the method comprising: providing a set of user data items to an EHR NFT service; and receiving, from the EHR NFT service, a minted EHR-NFT based on verification of the set of user data items by the EHR NFT service.

Example B02 includes the method of example B01 and/or some other example(s) herein, wherein the set of user data items are provided to the EHR NFT service via a user application (app), and the minted EHR-NFT is received from the EHR NFT service via the user app.

Example B03 includes the method of example B02 and/or some other example(s) herein wherein the user app is a client app, a wallet app, a payment app, a mobile app, a web app, or a hybrid app.

Example B04 includes the method of example B01 and/or some other example(s) herein, wherein the set of user data items are provided to the EHR NFT service via a healthcare provider (HP) platform, and the minted EHR-NFT is received from the EHR NFT service via the HP platform.

Example B05 includes the method of example B04 and/or some other example(s) herein, wherein the HP platform includes one or more of: hospital information system (HIS), hospital management system (HMS), health information management (HIM) system, one or more health informatics tools, health information technology, or one or more database management systems.

Example B06 includes a method for providing an electronic health record (EHR) non-fungible token (NFT) service, comprising: receiving, by the EHR NFT service, a set of user data items associated with a user; verifying, by the EHR NFT service, each user data item of the set of user data items; generating, by the EHR NFT service, a set of hashes, wherein each hash in the set of hashes corresponds to a verified user data item in the set of user data items; minting, by the EHR NFT service, an EHR NFT that includes each hash in the set of hashes; and updating, by the EHR NFT service, a smart contract to include the EHR NFT.

Example B07 includes the method of example B06 and/or some other example(s) herein, wherein the method includes: receiving a subset of user data items of the set of user data items via a user application (app); and sending the minted EHR NFT to the user app.

Example B08 includes the method of examples B06-B07 and/or some other example(s) herein, wherein the method includes: receiving a subset of user data items in the set of user data items from a healthcare provider (HP) platform; and sending the minted EHR NFT to the HP platform.

Example B09 includes the method of examples B06-B08 and/or some other example(s) herein, wherein the method includes: generating a block for inclusion in a blockchain; and adding the generated block to the blockchain.

Example B10 includes the method of example B09 and/or some other example(s) herein, wherein the block includes the minted EHR NFT.

Example B11 includes the method of examples B09-B10 and/or some other example(s) herein, wherein the block includes the set of hashes.

Example B12 includes the method of examples B09-B10 and/or some other example(s) herein, wherein the block includes an individual hash of the set of hashes.

Example B13 includes the method of examples B09-B12 and/or some other example(s) herein, wherein the block includes a hash of a previous block in the blockchain.

Example B14 includes the method of examples B09-B13 and/or some other example(s) herein, wherein the block does not include the set of user data items.

Example B15 includes the method of examples B09-B14 and/or some other example(s) herein, wherein the method includes: storing the set of user data items in a private database separate from the blockchain.

Example B16 includes the method of examples B06-B15 and/or some other example(s) herein, wherein the generated smart contract includes conditions, parameters, or criteria for detecting EHR updates.

Example B17 includes the method of example B16 and/or some other example(s) herein, wherein the method includes: detecting the EHR updates based on the conditions, parameters, or criteria in the smart contract, wherein the EHR updates include changes to existing EHR data associated with the user, newly generated EHR data associated with the user, or anomalous transactions between two or more HPs; and sending a notification to one or more HP platforms defined in the smart contract, wherein the notification indicates the detected EHR updates.

Example B18 includes the method of examples B16-B17 and/or some other example(s) herein, wherein the method includes: receiving, by the EHR NFT service, one or more EHR policies from one or more HPs; generating, by the EHR NFT service, a set of real-time codes from the EHR policies; and generating, by the EHR NFT service, the conditions, parameters, or criteria in the smart contract based on the set of real-time codes.

Example B19 includes the method of example B18 and/or some other example(s) herein, wherein the real-time codes are code snippets in the smart contract configured to monitor for the EHR updates.

Example B20 includes the method of example B19 and/or some other example(s) herein, wherein the real-time codes are code snippets in the smart contract configured to generate one or more notifications based on the monitored EHR updates.

Example B21 includes the method of example B19-B20 and/or some other example(s) herein, wherein the real-time codes are code snippets configured to submit, to one or more trained machine learning (ML) models, inference data including a subset of user data items in the set of user data items; and code snippets configured to receive a predicted diagnosis from the one or more trained ML models based on the inference data.

Example B22 includes the method of examples B01-B21 and/or some other example(s) herein, wherein the method includes: submitting the EHR NFT to a set of selected HPs; and receiving, from each HP in the set of selected HPs, EHR data associated with the user.

Example B23 includes the method of examples B01-B22 and/or some other example(s) herein, wherein the set of user data items includes one or more of: personal information, confidential information, sensitive information, one or more identity documents, authentication or authorization credentials, biometric data, electronic health records, knowledge-based authentication (KBA) data, social media profile data, or user system data of a user system used to provide the set of user data items to the EHR NFT service.

Example B24 includes the method of example B23 and/or some other example(s) herein, wherein the user system data includes one or more of: one or more user identifiers (IDs) or addresses, one or more timestamps, one or more digital certificates, geolocation information associated with access of the EHR NFT service, a client app ID, an app type of the app, app vendor, version of the app, app session ID of the app, user agent string, operating system (OS) type, an OS version, OS vendor, network session ID, device ID, serial number, product ID, a cookie ID, domain name, a rendering engine type, a rendering engine version, a device type of the user system, a device manufacturer, hardware platform type, one or more device serial numbers, a device fingerprint, system information of the user system, a screen or display resolution of the user system, or metadata.

Example B25 includes the method of examples B01-B24 and/or some other example(s) herein, wherein the set of user data items includes an NFT identity (ID) associated with the user.

Example B26 includes the method of example B25 and/or some other example(s) herein, wherein the NFT ID is generated by an NFT ID service that is separate from the EHR NFT service.

Example B27 includes the method of example B25 and/or some other example(s) herein, wherein the NFT ID generated by the EHR NFT service.

Example B28 includes the method of examples B01-B27 and/or some other example(s) herein, wherein the method is performed by a client computing device or a set of server computing devices.

Example B29 includes the method of examples B08-B28 and/or some other example(s) herein, wherein the HP platform includes one or more of: hospital information system (HIS), hospital management system (HMS), health information management (HIM) system, one or more health informatics tools, health information technology, or one or more database management systems.

Example B30 includes the method of examples B01-B29 and/or some other example(s) herein, wherein the method is performed by a client computing device or a set of server computing devices.

Example B31 includes the method of examples B01-B30 and/or some other example(s) herein, wherein the EHR NFT service is implemented by a set of servers, wherein the set of servers are part of a cloud computing service, an edge computing network, and/or a cellular communications network.

Example Z00 includes the method of examples A01-A12, B01-B31, and/or some other example(s) herein, wherein the method includes any one or more aspects as shown and described herein. Example Z01 includes one or more computer readable media comprising instructions, wherein execution of the instructions by processor circuitry is to cause the processor circuitry to perform the method examples A01-A12, B01-B31, and/or some other example(s) herein. Example Z02 includes a computer program comprising the instructions of example Z01 and/or some other example(s) herein. Example Z03 includes an Application Programming Interface defining functions, methods, variables, data structures, and/or protocols for the computer program of example Z02 and/or some other example(s) herein. Example Z04 includes an apparatus comprising circuitry loaded with the instructions of example Z01 and/or some other example(s) herein. Example Z05 includes an apparatus comprising circuitry operable to run the instructions of example Z01 and/or some other example(s) herein. Example Z06 includes an integrated circuit comprising one or more of the processor circuitry and the one or more computer readable media of example Z01 and/or some other example(s) herein. Example Z07 includes a computing system comprising the one or more computer readable media and the processor circuitry of example Z01 and/or some other example(s) herein. Example Z08 includes an apparatus comprising means for executing the instructions of example Z01 and/or some other example(s) herein. Example Z09 includes a signal generated as a result of executing the instructions of example Z01 and/or some other example(s) herein. Example Z10 includes a data unit generated as a result of executing the instructions of example Z01 and/or some other example(s) herein. Example Z11 includes the data unit of example Z10 and/or some other example(s) herein, the data unit is a datagram, network packet, data frame, data segment, a Protocol Data Unit (PDU), a Service Data Unit (SDU), a message, or a database object. Example Z12 includes a signal encoded with the data unit of examples Z10-Z11 and/or some other example(s) herein. Example Z13 includes an electromagnetic signal carrying the instructions of example Z01 and/or some other example(s) herein. Example Z14 includes an apparatus comprising means for performing the method examples A01-A12, B01-B31, and/or some other example(s) herein.

### 4. Terminology

For the purposes of the present document, the terminology discussed in '074, '081, and '396 may be applicable to the examples and embodiments discussed herein. As used herein, the singular forms “a,” “an” and “the” are intended to include plural forms as well, unless the context clearly indicates otherwise. It will be further understood that the terms “comprises” and/or “comprising,” when used in this specification, specific the presence of stated features, integers, steps, operations, elements, and/or components, but do not preclude the presence or addition of one or more other features, integers, steps, operation, elements, components, and/or groups thereof. The phrase “A and/or B” means (A), (B), or (A and B). For the purposes of the present disclosure, the phrase “A, B, and/or C” means (A), (B), (C), (A and B), (A and C), (B and C), or (A, B and C). The phrase “X(s)” means one or more X or a set of X. The description may use the phrases “in an embodiment,” “In some embodiments,” “in one implementation,” “In some implementations,” “in some examples”, and the like, each of which may refer to one or more of the same or different embodiments, implementations, and/or examples. Furthermore, the terms “comprising,” “including,” “having,” and the like, as used with respect to the present disclosure, are synonymous.

The terms “configuration”, “policy”, “ruleset”, and/or “operational parameters”, at least in some examples refer to a machine readable information object that contains instructions, conditions, parameters, criteria, data, metadata, and/or other information that is/are relevant to a component, device, system, network, service producer, service consumer, and/or other element/entity.

The term “information object” or “InOb” at least in some examples refers to a data structure or piece of information, definition, or specification that includes a name to identify its use in an instance of communication. Additionally or alternatively, the term “information object” or “InOb” at least in some examples refers to a configuration item that displays information in an organized form. Additionally or alternatively, the term “information object” or “InOb” at least in some examples refers to an abstraction of a real information entity and/or a representation and/or an occurrence of a real-world entity. Additionally or alternatively, the term “information object” or “InOb” at least in some examples refers to a data structure that contains and/or conveys information or data. Each of the data formats may also define the language, syntax, vocabulary, and/or protocols that govern information storage and/or exchange. Examples of the data formats that may be used for any of the InObs discussed herein may include any of those mentioned in '074 and/or '081.

The term “data format”, “file format”, and/or “format” at least in some examples refers to a data structure, specification, and/or standard that defines the language, syntax, vocabulary, and/or protocols that govern information storage and/or exchange. Examples of the data formats that may be used for any of the InObs discussed herein include: Abstract Syntax Notation One (ASN.1), Accelerated Mobile Pages Script (AMPscript), Accredited Standards Committee X12 (ASC) X12, Apex®, Backus-Naur Form (BNF), extended BNF, Bencode, BSON, ColdFusion Markup Language (CFML), Cascading Stylesheets (CSS), comma-separated values (CSV), Control Information Exchange Data Model (C2IEDM), DARPA Agent Markup Language (DAML), Document Type Definition (DTD), Electronic Data Interchange (EDI), Extensible Data Notation (EDN), Extensible Markup Language (XML), Efficient XML Interchange (EXI), Extensible Stylesheet Language (XSL), Free Text (FT), Fixed Word Format (FWF), Cisco® Etch, Franca, Geography Markup Language (GML), Guide Template Language (GTL), Handlebars template language, Hypertext Markup Language (HTML), Interactive Financial Exchange (IFX), Keyhole Markup Language (KML), JAMscript, JavaScript, Java Script Object Notion (JSON), JSON Schema Language, Jinja, LaTeX, markdown, MessagePack™, Mustache template language, National Council for Prescription Drug Programs (NCPDP) standards, Ontology Interchange Language (OIL), Open Service Interface Definition, Open Financial Exchange (OFX), Precision Graphics Markup Language (PGML), Google® Protocol Buffers (protobuf), Quicken® Financial Exchange (QFX), Regular Language for XML Next Generation (RelaxNG) schema language, regular expressions, Resource Description Framework (RDF) schema language, RESTful Service Description Language (RSDL), Scalable Vector Graphics (SVG), Schematron, Tactical Data Link (TDL) format (e.g., J-series message format for Link 16; JREAP messages; Multifuction Advanced Data Link (MADL), Integrated Broadcast Service/Common Message Format (IBS/CMF), Over-the-Horizon Targeting Gold (OTH-T Gold), Variable Message Format (VMF), United States Message Text Format (USMTF), and any future advanced TDL formats), VBScript, Web Application Description Language (WADL), Web Ontology Language (OWL), Web Services Description Language (WSDL), wiki markup or Wikitext, Wireless Markup Language (WML), extensible HTML (XHTML), XPath, XQuery, XML DTD language, XML Schema Definition (XSD), XML Schema Language, XSL Transformations (XSLT), YAML (“Yet Another Markup Language” or “YANL Ain′t Markup Language”), and/or any other data format, schema language, and/or language discussed elsewhere herein.

The term “data set” or “dataset” at least in some examples refers to a collection of data; a “data set” or “dataset” may be formed or arranged in any type of data structure. In some examples, one or more characteristics can define or influence the structure and/or properties of a dataset such as the number and types of attributes and/or variables, and various statistical measures (e.g., standard deviation, kurtosis, and/or the like). The term “data structure” at least in some examples refers to a data organization, management, and/or storage format. Additionally or alternatively, the term “data structure” at least in some examples refers to a collection of data values, the relationships among those data values, and/or the functions, operations, tasks, and the like, that can be applied to the data. Examples of data structures include primitives (e.g., Boolean, character, floating-point numbers, fixed-point numbers, integers, reference or pointers, enumerated type, and/or the like), composites (e.g., arrays, records, strings, union, tagged union, and/or the like), abstract data types (e.g., data container, list, tuple, associative array, map, dictionary, set (or dataset), multiset or bag, stack, queue, graph (e.g., tree, heap, and the like), and/or the like), routing table, symbol table, quad-edge, blockchain, purely-functional data structures (e.g., stack, queue, (multi) set, random access list, hash consing, zipper data structure, and/or the like.

The term “electronic health record” or “EHR” at least in some examples refers to a systematized collection of patient and population electronically stored health information in a digital format. Additionally or alternatively, the term “electronic health record” or “EHR” at least in some examples refers to a health record and/or personal health record (PHR) in electronic form. Additionally or alternatively, the term “electronic health record” or “EHR” at least in some examples refers to a collection of a patient's stored health information in a digital format. As examples, EHRs include a range of data, including demographics, medical history, progress notes, problems, medications, allergies, vital signs, immunization status, laboratory test results, radiology images, vital signs, personal statistics/data (e.g., age, weight, and the like), and billing information. For purposes of the present disclosure, the terms “electronic health record”, “electronic medical record”, “electronic patient record”, “personal health record”, and/or the like may be used interchangeably, even though these terms may refer to different concepts in some cases or contexts. The term “personal health record” or “PHR” at least in some examples refers to a health record where health data and other information related to the care of a patient is maintained by the patient. Additionally or alternatively, the term “personal health record” or “PHR” at least in some examples refers to an electronic application for recording personal medical data that an individual patient controls and may make available to health providers.

The term “healthcare provider”, “health care provider”, or “HP” at least in some examples refers to an individual professional, facility, and/or organization licensed to provide healthcare diagnosis and treatment services including medication, surgery, medical devices, and the like. Examples of HPs include physicians, dentists, advanced practice providers (APPs) (e.g., physician assistants, nurses, pharmacists, midwives, chiropractors, social workers, psychologists, and/or the like), allied health professionals (e.g., technicians, therapists, hygienists, medical/dental assistants, nutritionists, scribes, counselors, physiologists, interpreters, radiation scientists, midwives, paramedics, pathologists, radiographers, sonographers, and/or the like), health professionals, individual hospitals, hospital networks, healthcare system, medical group, medical practice, clinics, and/or the like. Additionally or alternatively, HPs can also include governmental agencies, such as the World Health Organization (WHO), United States Department of Health and Human Services (HHS), UK National Health Service (NHS), Health Canada, European Centre for Disease Prevention and Control (ECDC), and/or the like.

The term “knowledge-based assessment”, “knowledge-based authentication”, or “KBA” at least in some examples refers to verification and/or authentication processes or procedures, which requires the knowledge of private information (or personal data) to prove that the entity/element providing the identifying information is the actual owner of the corresponding identity. In some examples, KBA-generated questions are static KBAs or dynamic KBAs. The term “static KBAs” at least in some examples refers to a pre-agreed set of KBA data and/or shared secrets, such as place of birth, mother's maiden name, name of first pet, and/or the like. The term “dynamic KBAs” at least in some examples refers to KBA-questions generated from a wider base of personal information, such as account numbers, loan amounts, tax payment amounts, and/or the like. Additionally or alternatively, the term “dynamic KBAs” at least in some examples refers to KBA-questions that are generated based on changing parameters or conditions, such as previously supplied KBA questions, timing parameters, and/or the like.

The term “blockchain” at least in some examples refers to a list of records (referred to as “blocks”) that are linked together in some way, usually using cryptography. The term “block” as used in “blockchain” at least in some examples refers to a batch of transactions with a reference to a previous block (e.g., a hash of the previous block) in a blockchain thereby linking the block to the previous block. In various implementations, each block in a blockchain contains a cryptographic hash of a previous block, a timestamp, and transaction data (e.g., represented as a Merkle tree). Additionally or alternatively, the term “blockchain” at least in some examples refers to a digital database containing information, such as records of financial or other transactions, which can be simultaneously used and shared within a large decentralized, publicly accessible network. Additionally or alternatively, the term “blockchain” at least in some examples refers to a shared, immutable ledger that facilitates the process of recording transactions and tracking assets (tangible and intangible) in a network. In some examples, a blockchain includes a list of records, called blocks, that are linked together using cryptography. The term “blockchain mining” or “mining” at least in some examples refers to a process (e.g., computer computations) by which transactions are validated and verified for inclusion in a blockchain.

The terms “consensus algorithm”, “consensus protocol”, “consensus mechanism”, and/or “consensus” at least in some examples refers to one or more coordinating processes that allows distributed computing applications and/or multi-agent systems to agree on a data value that is needed during computation. Examples of consensus algorithms include proof of work (PoW), reusable PoW, proof of useful work, proof of stake (POS), proof of space, proof of authority, Specialized Proofs of Confidential Knowledge (SPoCKs), Delegated Proof of Stake (DPOS), Byzantine fault tolerance (BFT), BFT-DPOS, Transaction as Proof of Stake (TaPoS), Paxos, distributed lock services (e.g., Chubby provided by Google®), lockstep protocols, Byzantine agreement protocol (BAP), quantum BAP, and/or the like.

The term “consortium blockchain” at least in some examples refers to a group of private blockchains, each owned by individual institutions that permit the sharing of information for various purposes, such as improving/streamlining workflows, transparency, and/or accountability. Additionally or alternatively, the term “consortium blockchain” at least in some examples refers to a hybrid blockchain networks that combines public and private blockchain network features. Additionally or alternatively, the term “consortium blockchain” at least in some examples refers to a permissioned blockchain networks governed by a group of organizations that have decided to share a ledger among themselves for transactions. Additionally or alternatively, the term “consortium blockchain” can also be referred to as a “federated blockchain”.

The term “decentralized application”, “decentralized app”, “Dapp”, “Ðapp”, or “dApp” at least in some examples refers to an application that can operate autonomously, for example, through the use of smart contracts, that run on a decentralized computing platform, blockchain, or other distributed ledger system. Additionally or alternatively, the term “decentralized application”, “decentralized app”, “Dapp”, “Ðapp”, or “dApp” at least in some examples refers to an application configured to generate and distribute tokens or NFTs according to a standard algorithm and/or a set of criteria.

The term “Ethereum” at least in some examples refers to a decentralized, open-source blockchain with smart contract functionality. The term “Ether”, “ETH”, or “Ξ” at least in some examples refers to the native cryptocurrency of the Ethereum platform. The term “Ethereum Improvement Proposals” or “EIPs” at least in some examples refers to standards for the Ethereum platform, including core protocol specifications, client APIs, and contract standards.

The term “Ethereum Virtual Machine” or “EVM” at least in some examples refers to a runtime environment for transaction execution in Ethereum. Additionally or alternatively, the term “Ethereum Virtual Machine” or “EVM” at least in some examples refers to a global virtual computer whose state every participant on the Ethereum network stores and agrees on; where any participant can request the execution of arbitrary code on the EVM; code execution changes the state of the EVM. Additional aspects of Ethereum, Ether, and EVMs are discussed in Ethereum Development Documentation, ETHEREUM.ORG (18 Oct. 2018), https://ethereum.org/en/developers/docs/(“[EthereumDoc]”), Ethereum Glossary, ETHEREUM.ORG (23 Feb. 2022), https://ethereum.org/en/glossary/, Takenobu T., Ethereum EVM illustrated: exploring some mental models and implementations, rev. 0.01.1 (March 2018), http://takenobu-hs.github.io/downloads/ethereum_evm_illustrated.pdf, and Dr. Gavin Wood, Ethereum: A secure Decentralised Generalised Transaction Ledger, Berlin version b8ffc51 (21 Feb. 2022), https://ethereum.github.io/yellowpaper/paper.pdf (“[Ethereum-Yellow-Paper]”), the contents of each of which are hereby incorporated by reference in their entireties and for all purposes.

The terms “flow blockchain”, “flow framework”, or simply “flow” at least in some examples refers to a node pipeline that may be used to verify blockchain transactions as discussed in Hentschel et al., Flow: Separating Consensus and Compute, arXiv: 1909.05821v1 [cs.DC], 21 pages (12 Sep. 2019); Hentschel et al., Flow: Separating Consensus and Compute—Execution Verification—, arXiv: 1909.05832v1 [cs.DC] (12 Sep. 2019); Hentschel et al., Flow: Separating Consensus and Compute—Block Formation and Execution—, arXiv: 2002.07403v1 [cs.DC] (18 Feb. 2020); Flow Primer, FLOW BLOCKCHAIN (24 Sep. 2020), https://flow.com/primer; FLOW Token Economics, FLOW BLOCKCHAIN (23 Sep. 2020), https://flow.com/flow-token-economics; Mackenzie 10thfloor, How to Launch a Fungible Token on Flow, FLOW DEVELOPERS (22 Sep. 2022), https://developers.flow.com/flow/fungible-tokens; [Flow-NFT]; Bastian Müller (“turbolent”), [Cadence]; and Lafrance et al., Incentives in a Multi-Role Blockchain, 16 pages (21 Sep. 2020) (collectively referred to herein as “[Flow]”), the contents of each of which are hereby incorporated by reference in their entireties and for all purposes.

The term “gas” at least in some examples refers to a unit that measures the amount of computational effort required to execute specific operations on the Ethereum network. Additionally or alternatively, the term “gas” at least in some examples refers to a limit on the amount of work that is needed to execute a transaction. The term “gas price” or “gas fee” at least in some examples refers to the fee required to conduct a transaction successfully in terms of computational resources, computational complexity, and/or currency.

The term “housekeeping” at least in some examples refers to an entry or exit routine appended to a user-written block of code (such as a routine, subroutine, function, smart contract, and/or the like) at its entry and exit. Additionally or alternatively, the term “housekeeping” at least in some examples refers to any automated or manual process whereby a computer is cleaned up after usage (e.g., freeing resources such as virtual memory or the like, saving and/or restoring a program state or context, initializing local variables, garbage collection processes, data conversion, backing up data, disk maintenance processes, and the like).

The term “InterPlanetary File System” or “IPFS” at least in some examples refers to a protocol and peer-to-peer network for storing and sharing data in a distributed file system. IPFS uses content-addressing to uniquely identify each file in a global namespace connecting all computing devices. Additional aspects of IPFS are discussed in Interplanetary File System (IPFS) Documentation, IPFS DOCS (2018), https://docs.ipfs.io/(“[IPFS]”), the contents of which is hereby incorporated by reference in its entirety and for all purposes.

The term “Merkle tree” or “hash tree” at least in some examples refers to a tree data structure in which every leaf node is labelled with the cryptographic hash of a data block, and every node that is not a leaf node (e.g., a branch node, inner node, or inode) is labelled with the cryptographic hash of the labels of its child nodes. Additionally or alternatively, “hash tree” at least in some examples is a generalization of a hash list and a hash chain.

The term “minting” at least in some examples refers to a process of turning digital data into a digital asset, such as a token or non-fungible token (NFT), and may involve adding the digital asset to a blockchain or other distributed ledger. In some examples, “minting” is performed according to [EIP-721], [EIP-1155], Lockyer et al., EIP-998: ERC-998 Composable Non-Fungible Token Standard [DRAFT], ETHEREUM IMPROVEMENT PROPOSALS, no. 998 (July 2018), https://eips.ethereum.org/EIPS/eip-998 (“[EIP-998]”), Buterin et al., EIP-1559: Fee market change for ETH 1.0 chain, ETHEREUM IMPROVEMENT PROPOSALS, no. 1559 (April 2019), https://eips.ethereum.org/EIPS/eip-1559 (“[EIP-1559]”), Marro et al., EIP-5375: NFT Author Information and Consent, ETHEREUM IMPROVEMENT PROPOSALS, no. 5375 (July 2022), https://eips.ethereum.org/EIPS/eip-5375 (“[EIP-5375]”), and/or Zainan Victor Zhou, EIP-5679: Token Minting and Burning, ETHEREUM IMPROVEMENT PROPOSALS, no. 5679, (September 2022), https://eips.ethereum.org/EIPS/eip-5679 (“[EIP-5679]”), the contents of each of which are hereby incorporated by reference in their entireties and for all purposes.

The term “non-fungible token” or “NFT” at least in some examples refers to a non-interchangeable unit of data. Additionally or alternatively, the term “non-fungible token” or “NFT” at least in some examples refers to a non-interchangeable unit of data that provides or otherwise acts as a certificate of authenticity or proof of ownership of some physical or virtual item or object. NFTs may be stored on a blockchain or other form of digital ledger, and sold or traded. NFTs may be associated with digital files such as photos, videos, audio, virtual property or virtual assets, and the like. Moreover, because individual NFTs are uniquely identifiable, NFTs differ from blockchain cryptocurrencies, such as Bitcoin, Ether, and the like. Additional aspects of NFTs are discussed in Entriken et al., EIP-721: Non-Fungible Token Standard, ETHEREUM IMPROVEMENT PROPOSALS, no. 721 (24 Jan. 2018), https://eips.ethereum.org/EIPS/eip-721 (“[EIP-721]”) and Reitwießner et al., EIP-165: Standard Interface Detection, ETHEREUM IMPROVEMENT PROPOSALS, no. 165 (23 Jan. 2018), https://eips.ethereum.org/EIPS/eip-165 (“[EIP-165]”), Burks et al., EIP-2981: NFT Royalty Standard, ETHEREUM IMPROVEMENT PROPOSALS, no. 2981 (September 2020), https://eips.ethereum.org/EIPS/eip-2981 (“[EIP-2981]”), Anders et al., EIP-4907: Rental NFT, an Extension of EIP-721, ETHEREUM IMPROVEMENT PROPOSALS, no. 4907 (March 2022), https://eips.ethereum.org/EIPS/eip-4907 (“[EIP-4907]”), Lance et al., EIP-5006: Rental NFT, NFT User Extension [DRAFT], ETHEREUM IMPROVEMENT PROPOSALS, no. 5006 (April 2022), https://eips.ethereum.org/EIPS/eip-5006 (“[EIP-5006]”), Anders et al., EIP-5007: Time NFT, EIP-721 Time Extension [DRAFT], ETHEREUM IMPROVEMENT PROPOSALS, no. 5007 (April 2022), https://eips.ethereum.org/EIPS/eip-5007 (“[EIP-5007]”), Siemens et al., Flow Non-Fungible Token Standard, FLOW BLOCKCHAIN (20 Dec. 2021) https://github.com/onflow/flow-nft/blob/master/README.md (“[Flow-NFT]”), and/or Karslen et al., Non-Fungible Token Metadata, FLOW IMPROVEMENT PROPOSAL, no. 636 (6 Dec. 2021), the contents of each of which are hereby incorporated by reference in their entireties and for all purposes.

The term “on-chain transaction” at least in some examples refers to a transaction that occurs and is considered valid when the blockchain is modified. Additionally or alternatively, the term “on-chain transaction” at least in some examples refers to a transaction that is carried out on a blockchain or blockchain network. In some examples, the term “on-chain transaction” may be synonymous with the term “transaction”.

The term “off-chain transaction” at least in some examples refers to a transaction that takes place or takes value outside of a blockchain. Additionally or alternatively, the term “off-chain transaction” at least in some examples refers to a transaction that is confirmed outside of a blockchain network. Additionally or alternatively, the term “off-chain transaction” at least in some examples refers to a transaction that involves some of the transaction-related work from a blockchain ecosystem, which can later be integrated back into a blockchain.

The term “smart contract” at least in some examples refers to a set of self-executing code. Additionally or alternatively, the term “smart contract” at least in some examples refers to a program, application, set of trigger functions, or transaction protocol that is intended to automatically execute, control, or document relevant events and actions according to some predefined criteria or conditions such as those defined in a contract, agreement, or policy. Additional aspects of smart contracts are discussed in Mudge et al., EIP-173: Contract Ownership Standard, ETHEREUM IMPROVEMENT PROPOSALS, no. 173 (7 Jun. 2018), https://eips.ethereum.org/EIPS/eip-173 (“[EIP-173]”), Radomski et al., EIP-1155: Multi Token Standard, ETHEREUM IMPROVEMENT PROPOSALS, no. 1155 (17 Jun. 2018), https://eips.ethereum.org/EIPS/eip-1155 (“[EIP-1155]”), Murray et al., EIP-1167: Minimal Proxy Contract, ETHEREUM IMPROVEMENT PROPOSALS, no. 1167 (June 2018), https://eips.ethereum.org/EIPS/eip-1167 (“[EIP-1167]”), Giordano et al., EIP-1271: Standard Signature Validation Method for Contracts, ETHEREUM IMPROVEMENT PROPOSALS, no. 1271 (July 2018), https://eips.ethereum.org/EIPS/eip-1271 (“[EIP-1271]”), Enterprise Ethereum Alliance Client Specification v7, EEA Editor's Draft, version 7 (10 Feb. 2022), https://entethalliance.github.io/client-spec/spec.html (“[EEA-CS7]”), Solidity Documentation, release 0.8.18, revision c1040815 (8 Sep. 2022) (“[Solidity]”), Vyper Team, Vyper Documentation, release v0.3.8, revision 1cd6f3db (15 Dec. 2022), FuelLaps™, Yul+, version 0.2.3 (10 Oct. 2020), https://github.com/FuelLabs/yulp; The Cadence Programming Language, FLOW DEVELOPERS (17 Mar. 2021), https://developers.flow.com/cadence/language/index (“[Cadence]”), and Contracts, OPEN ZEPPLIN|DOCS, version 4.x (2022), https://docs.openzeppelin.com/contracts/4.x/(“[OZContracts]”), the contents of each of which are hereby incorporated by reference in their entireties and for all purposes. In some examples, one or more smart contracts can be implemented as “diamonds” as discussed in Mudge, EIP-2535: Diamonds, Multi-Facet Proxy, ETHEREUM IMPROVEMENT PROPOSALS, no. 2535 (February 2020), https://eips.ethereum.org/EIPS/eip-2535 (“[EIP-2535]”), the contents of which are hereby incorporated by reference in its entirety and for all purposes.

The term “token” at least in some examples refers to a software and/or hardware object or element that represents the right or ability to perform an operation. Additionally or alternatively, the term “token” at least in some examples refers to a software and/or hardware object or element that references or maps to another data item, which may be done through a tokenization system. The term “security token”, “authentication token”, “cryptographic token”, or the like at least in some examples refers to a software object and/or a physical device used for computer authentication. The term “tokenization” at least in some examples refers to a process for substituting a data item with another equivalent data item (e.g., a “token”) that has no extrinsic or exploitable meaning or value, or at least a different meaning or value than the original data item. Additional aspects of tokens are discussed in Vogelsteller et al., EIP-20: Token Standard, ETHEREUM IMPROVEMENT PROPOSALS, (19 no. 20 Nov. 2015), https://eips.ethereum.org/EIPS/eip-20 (“[EIP-20]”), Dafflon et al., EIP-777: Token Standard, ETHEREUM IMPROVEMENT PROPOSALS, no. 777 (20 Nov. 2017), https://eips.ethereum.org/EIPS/eip-777 (“[EIP-777]”), StartfundInc, EIP-5528: Refundable Fungible Token, ETHEREUM IMPROVEMENT PROPOSALS, no. 5528 (August 2022), https://eips.ethereum.org/EIPS/eip-5528 (“[EIP-5528]”), and/or Igualada et al., Fungible Token Metadata, FLOW IMPROVEMENT PROPOSAL, no. 1087 (16 Aug. 2022), the contents of each of which are hereby incorporated by reference in their entireties and for all purposes. In some examples, the term “token” and/or “NFT” can include semi-fungible tokens as discussed in Wang et al., EIP-3525: Semi-Fungible Token, ETHEREUM IMPROVEMENT PROPOSALS, no. 3525 (December 2020), https://eips.ethereum.org/EIPS/eip-3525 (“[EIP-3525]”) and/or Zhu et al., EIP-5727: Semi-Fungible Soulbound Token [DRAFT], ETHEREUM IMPROVEMENT PROPOSALS, no. 5727 (September 2022), https://eips.ethereum.org/EIPS/eip-5727 (“[EIP-5727]”), the contents of each of which are hereby incorporated by reference in their entireties and for all purposes.

The term “transaction” at least in some examples refers to a unit of work performed within a computing system and/or a database management system. Additionally or alternatively, the term “transaction” at least in some examples refers to information processing that is divided into individual, indivisible operations. Additionally or alternatively, the term “transaction” at least in some examples refers to the physical and/or electronic exchange or transfer of assets or objects. Additionally or alternatively, the term “transaction” at least in some examples refers to the electronic exchange of information between two parties to carry out financial or administrative activities related to health care (e.g., a transaction may involve a health care provider sending a claim to a health plan to request payment for medical services). Additionally or alternatively, the term “transaction” at least in some examples refers to a request to execute operations on a blockchain that change the state of one or more accounts. The term “private transaction” at least in some examples refers to a transaction where some information about the transaction, such as the payload data, or the sender or the recipient, is only available to the subset of parties privy to that transaction.

The term “wallet” or “digital wallet” at least in some examples refers to an electronic device, online/web service, and/or software application that allows a party to make electronic transaction with another party. Additionally or alternatively, the term “wallet” or “digital wallet” at least in some examples refers to an electronic device, online/web service, and/or software application used to store credentials (e.g., cryptographic private keys) that are used to perform transactions and/or access an account. Additional aspects of wallets are discussed in [EEA-CS7].

Aspects of the inventive subject matter may be referred to herein, individually and/or collectively, merely for convenience and without intending to voluntarily limit the scope of this application to any single aspect or inventive concept if more than one is in fact disclosed. Thus, although specific aspects have been illustrated and described herein, it should be appreciated that any arrangement calculated to achieve the same purpose may be substituted for the specific aspects shown. This disclosure is intended to cover any and all adaptations or variations of various aspects. Combinations of the above aspects and other aspects not specifically described herein will be apparent to those of skill in the art upon reviewing the above description.

1. An apparatus for providing an electronic health record (EHR) non-fungible token (NFT) service, the apparatus comprising:
memory circuitry; and
processor circuitry connected to the memory circuitry, wherein the processor circuitry is to:
receive a set of user data items associated with a user;
verify each user data item of the set of user data items;
generate a set of hashes, wherein each hash in the set of hashes corresponds to a verified user data item in the set of user data items;
mint an EHR NFT that includes each hash in the set of hashes; and
update a smart contract to include the EHR NFT.

2. The apparatus of claim 1, wherein the processor circuitry is to:
receive a subset of user data items in the set of user data items from a healthcare provider (HP) platform;
receive another subset of user data items in the set of user data items from a user application (app); and
send the minted EHR NFT to the HP platform or the user app.

3. The apparatus of claim 1, wherein the processor circuitry is to:
generate a block for inclusion in a blockchain, wherein the block includes the minted EHR NFT or the set of hashes; and
add the generated block to the blockchain after verification of the generated block.

4. The apparatus of claim 3, wherein the block includes a hash of a previous block in the blockchain, and the block does not include the set of user data items.

5. The apparatus of claim 3, wherein the processor circuitry is to:
cause storage of the set of user data items in a private database separate from the blockchain.

6. The apparatus of claim 1, wherein the generated smart contract includes conditions, parameters, or criteria for detecting EHR updates, and the processor circuitry is to:
detect the EHR updates based on the conditions, parameters, or criteria in the smart contract, wherein the EHR updates include changes to existing EHR data associated with the user, newly generated EHR data associated with the user, or anomalous transactions between two or more HPs; and
send a notification to one or more HP platforms defined in the smart contract, wherein the notification indicates the detected EHR updates.

7. The apparatus of claim 6, wherein the processor circuitry is to:
receive one or more EHR policies from one or more HPs;
generate a set of real-time codes from the EHR policies; and
generate the conditions, parameters, or criteria in the smart contract based on the set of real-time codes.

8. The apparatus of claim 7, wherein the real-time codes include:
code snippets configured to monitor for the EHR updates; and
code snippets configured to generate one or more notifications based on the monitored EHR updates.

9. The apparatus of claim 7, wherein the real-time codes include:
code snippets configured to submit, to one or more trained machine learning (ML) models, inference data including a subset of user data items in the set of user data items; and
code snippets configured to receive a predicted diagnosis from the one or more trained ML models based on the inference data.

10. The apparatus of claim 1, wherein the set of user data items includes an NFT identity (ID) associated with the user, wherein the NFT ID is generated by an NFT ID service that is separate from the EHR NFT service or the NFT ID generated by the EHR NFT service.

11. At least one computer-readable media (NTCRM) comprising instructions for providing an electronic health record (EHR) non-fungible token (NFT) service, wherein execution of the instructions is to cause a computing device to:
receive, from one or more data sources, a set of user data items associated with a user, wherein at least some of the user data items in the set of data items includes EHR data associated with the user;
verify each user data item of the set of user data items;
generate a set of hashes, wherein each hash in the set of hashes corresponds to a verified user data item in the set of user data items;
mint an EHR NFT that includes each hash in the set of hashes; and
update a smart contract to include the EHR NFT.

12. The NTCRM of claim 11, wherein execution of the instructions is to cause the computing device to:
generate a block for inclusion in a blockchain, wherein the block includes the minted EHR NFT or an individual hash in the set of hashes; and
add the generated block to the blockchain after verification of the generated block.

13. The NTCRM of claim 12, wherein the block includes a hash of a previous block in the blockchain, and the block does not include the set of user data items.

14. The NTCRM of claim 12, wherein execution of the instructions is to cause the computing device to:
cause storage of the set of user data items in a private database separate from the blockchain.

15. The NTCRM of claim 11, wherein the generated smart contract includes conditions, parameters, or criteria for detecting EHR updates, and execution of the instructions is to cause the computing device to:
detect the EHR updates based on the conditions, parameters, or criteria in the smart contract, wherein the EHR updates include changes to existing EHR data associated with the user, newly generated EHR data associated with the user, or anomalous transactions between two or more healthcare providers (HPs); and
send a notification to one or more HP platforms defined in the smart contract, wherein the notification indicates the detected EHR updates.

16. The NTCRM of claim 15, wherein execution of the instructions is to cause the computing device to:
receive one or more EHR policies from one or more HPs;
generate a set of real-time codes from the EHR policies; and
generate the conditions, parameters, or criteria in the smart contract based on the set of real-time codes.

17. The NTCRM of claim 16, wherein the real-time codes include:
code snippets configured to monitor for the EHR updates; and
code snippets configured to generate one or more notifications based on the monitored EHR updates.

18. The NTCRM of claim 16, wherein the real-time codes include:
code snippets configured to submit, to one or more trained machine learning (ML) models, inference data including a subset of user data items in the set of user data items; and
code snippets configured to receive a predicted diagnosis from the one or more trained ML models based on the inference data.

19. The NTCRM of claim 11, wherein the set of user data items includes one or more of: EHR data associated with the user, an NFT identity (ID) associated with the user, personal information, confidential information, sensitive information, one or more identity documents, authentication or authorization credentials, biometric data, knowledge-based authentication (KBA) data, social media profile data, or user system data of a user system used to provide the set of user data items to the EHR NFT service.

20. The NTCRM of claim 11, wherein the one or more data sources include one or more of: one or more HP platforms, a user system operated by the user, the EHR NFT service, one or more governmental databases, or one or more non-governmental organization databases.
